(19) United States
(12) Patent Application Publication
(10) Pub. No.: US 2011/0129458 A1
(43) Pub. Date: Jun. 2, 2011

(54) BINDING MOLECULES WITH MULTIPLE BINDING SITES, COMPOSITIONS COMPRISING THE SAME AND USES THEREOF

(75) Inventors: Edward Dolk, Utrecht (NL); Michael John Scott Saunders, Brussels (BE); Johannes Joseph Wilhelmus De Haard, Oudelande (NL); Renee De Bruin, Utrecht (NL)

(73) Assignee: Ablynx N.V., Ghent-Zwijnaarde (BE)

(21) Appl. No.: 12/676,705

(22) PCT Filed: Dec. 17, 2007
(86) PCT No.: PCT/EP2007/064063

§ 371 Date: Feb. 16, 2011

Related U.S. Application Data
(60) Provisional application No. 60/993,053, filed on Sep. 7, 2007.

(51) Int. Cl. A61K 39/395; C07K 16/18; C07K 16/00; C07H 21/00; C12N 5/10; C12P 21/00; A61P 37/02

(52) U.S. Cl. 424/133.1; 530/389.1; 530/387.1; 530/387.3; 536/23.1; 435/325; 435/69.6; 424/130.1

(57) ABSTRACT

The present invention relates to binding molecules, such as amino acid sequences with multiple antigen binding sites. In particular, the binding molecules of the present invention have at least two antigen binding sites that partially or fully overlap with each other and that are directed against at least two different naturally occurring binding molecules. The invention further relates to uses of such binders, for example in methods for inhibiting and/or blocking of the interaction between said at least two naturally occurring binding molecules and a third naturally occurring binding molecule.

### FIELD OF THE INVENTION

The present invention relates to binding molecules with multiple antigen binding sites (herein also referred to as the “binders of the invention” or the “multispecific binders of the invention”, such as e.g. “dual specific binders”, “triple specific binders”, “quadruple specific binders”, etc.). In particular, the binding molecules of the present invention have at least two antigen binding sites that partially or fully overlap with each other and that are, preferably, directed against at least two different naturally occurring binding molecules, such as a first and a second naturally occurring binding molecule (herein also referred to as the “first and second naturally occurring binding molecule”).

In a preferred aspect, the binding molecules (herein also referred to as “dual specific binders of the invention”) are directed against a first and a second naturally occurring binding molecule. The binders of the invention are preferably amino acid sequences or polypeptides (herein also referred to as the “amino acid sequences of the invention”). The invention further relates to uses of such binders, for example in methods for inhibiting and/or blocking of the interaction between said at least two naturally occurring binding molecules and a third naturally occurring binding molecule.

The invention further provides compounds or constructs (herein also referred as the “compounds of the invention”), and in particular polypeptides and proteins (herein also referred to as the “polypeptides of the invention”) comprising one or more of such binders or amino acid sequences of the invention. The invention further relates to nucleic acids (herein also referred to as “nucleic acids of the invention” or “nucleotides of the invention”) encoding the amino acid sequences or polypeptides of the invention; to methods for preparing the binders, amino acid sequences, compounds or polypeptides of the invention; to host cells expressing or capable of expressing the amino acid sequences or polypeptides of the invention; to compositions, and in particular to pharmaceutical compositions, that comprise the binders of the invention, amino acid sequences of the invention, compounds of the invention, polypeptides of the invention, nucleic acids of the invention and/or host cells; and to uses of the binders of the invention, amino acid sequences of the invention, compounds of the invention, polypeptides of the invention, nucleic acids of the invention, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

### BACKGROUND ART

In the past years various cell surface molecules have been identified that are implicated in cell responses, cell signaling and/or signal transduction cascades and that can induce certain biological pathways and/or biological mechanisms. These molecules, both cell type specific or more widely distributed molecules, are implicated in a variety of processes including cellular recognition, adhesion, induction and maintenance of proliferation, cytokine secretion, effector function, differentiation, proliferation, apoptosis, etc. The elucidation of these molecules and their role in cell signaling and different processes has provided potential new targets of therapeutic intervention, for example, by modulating, and in particular inhibiting or preventing cell signalling, modulating certain biological pathways, and/or modulating the biological mechanisms, responses and effects associated with such signalling or these pathways.

An example of such targets for therapeutic intervention are proteins of the TNF superfamily (Aggarwal, Nature Reviews Immunology 3: 747, 2003). This superfamily of proteins consists of 19 members that signal through 29 receptors. These ligands, while regulating normal functions such as immune responses, haematopoiesis and morphogenesis, have also been implicated in tumorgenesis, transplant rejection, septic shock, viral replication, bone resorption, rheumatoid arthritis and diabetes. Blockers of TNF have been approved for human use in treating TNF-linked autoimmune diseases. Whereas most ligands bind to a single receptor, others bind to more than one. For example, TRAIL binds to as many as five receptors (DR4, DR5, DVR1, DCR2 and OPG), whereas BAFF binds to three receptors, transmembrane activator and cyclophilin ligand interactor (TACT), B-cell maturation antigen (BMCA) and BAFFR (Aggarwal, 2003, FIG. 1). There is also evidence for crosstalk between receptors for different ligands of the TNF superfamily. It follows that, in order to achieve maximal therapeutic benefit, the interactions of all ligands with a particular receptor, or the interactions of a particular ligand with all its receptors should be inhibited at the same time. Therefore, for efficient therapy, various different binding molecules or binding molecules with multiple binding specificity are required.

Another example of possible targets for therapeutic intervention is a sub-family of the Receptor Tyrosin Kinases, the Eph family, comprised of 16 known Eph receptors (14 found in mammals) and 9 known ephrin ligands (8 found in mammals). The ability of the Eph receptor and ephrin ligand guidance system to position cells and modulate cell morphology reflects their various roles in development. These membrane anchored ligands and receptors are involved in bi-directional signaling (into both the receptor bearing cell and the ligand bearing cell. Eph receptors, first shown to be important regulators of axon path-finding and neuronal cell migration (Drescher et al., Cell 82: 359, 1995; Henkemeyer et al. Cell 86: 35, 1996), are now known to have roles in controlling a diverse array of other cell-cell interactions, including those of vascular endothelial cells (Wang et al., Cell 93: 741, 1998; Adams et al., Genes Dev. 13: 295, 1999; Gerety et al., Mol. Cell. 4: 403, 1999) and specialized epithelia (Orioli et al., EMBO J. 15: 6035, 1996; Flanagan and Vanderhaeghen Annu. Rev. Neurosci. 21: 309, 1998; Frisen et al, EMBO J. 18: 5159, 1999; Cowan et al., Neuron 26: 417, 2000). Ephrins and the ephrin receptor bidirectional signaling have been implicated in axonal guidance, angiogenesis and bone remodeling. Therapeutically, there is interest in antagonizing certain ephrin-Eph receptor signaling processes.

The ephrins and the Eph receptors are divided into two classes A and B based on their affinities for each other and sequence conservation. In general, the nine different EphA RTKs (EphA1-EphA9) bind promiscuously to, and are activated by, six A-ephrins (ephrinA1-ephrinA6), and the EphB subclass receptors (EphB1-EphB6 and, in some cases, EphA4) interact with three different B-ephrins (ephrinB1-ephrinB3). In order to achieve maximal therapeutic benefit, therefore, interactions of all ephrin ligands with a particular Eph receptor, or the interactions of a particular ephrin with all its Eph receptors should be inhibited at the same time. Accordingly, also here, for efficient therapy, various different binding molecules or binding molecules with multiple binding specificity will be needed.

The costimulatory molecules of the B7 superfamily are another example of possible targets for therapeutic intervention. The presence of co-stimulatory molecules on the APC is required (“signal 2”) alongside antigenic peptide in the context of the MHC molecule (“signal 1”) to obtain efficient stimulation of naïve antigen reactive T-cells. CD80, CD86, CD28, cytotoxic T lymphocyte antigen 4 (CTLA4), inducible costimulator (ICOS), programmed death 1 (PD-1), PD-L1, PD-L2 and OX 40 are used as targets to manipulate T-cells to slow the progression of autoimmune diseases, or to treat tumors through the increase in T-cell activation. CD80 (previously called B7-1) and CD86 (B7-2) are expressed on the membrane of activated antigen presenting cells (APC) such as dendritic cells, macrophages or B-cells. The presence of costimulatory molecules is sensed by counterreceptors on the surface of the T-cell. Selective blockade of the interaction of such costimulatory molecules with their cognate activating receptor (CD28) on the T-cell may therefore inhibit T-cell activation (Howard et al., Curr. Drug Targets Inflamm. Allergy 4: 85, 2005; Stuart and Racke, Expert Opinion Ther. Targets 6: 275, 2002).

Activated self-antigen directed T-cells are responsible for at least part of the tissue damage in autoimmune diseases such as rheumatoid arthritis or multiple sclerosis by virtue of their effector function, and indirectly for production of high-affinity self-reactive antibodies by providing “help” to B-cells. Thus, blockade of the interaction of CD80 and/or CD86 with CD28 can be therapeutic in autoimmune conditions. These principles have been firmly established in both animal models of human disease, as well as in man, by using either blocking monoclonal antibodies directed against CD80 or CD86, or using soluble forms of a counterreceptor (Stuart and Racke, 2002).

CD152 (previously known as CTLA4) is another counterreceptor on T-cells for both CD80 and CD86. Unlike CD28, however, interaction of CD152 with CD80 and/or CD86 does not lead to T-cell activation. CD152 is thought to interact with both CD80 and CD86 with a higher affinity than CD28, and may therefore serve as a decoy receptor for CD28, depriving the latter of its ligands and therefore indirectly decreasing T-cell activation (Collins et al., Immunity 17: 201, 2002). Alternatively, CD152 may also transduce a negative signal into the T-cell, leading to lower overall levels of T-cell activation. Regardless of the mechanism, the activity of CD152 signaling leads to a dampening of T-cell responses, especially late (48-72H) after T-cell stimulation when surface CD152 expression becomes high. Blocking CD152 signaling by the use of monoclonal antibodies blocking its interaction with CD80 and/or CD86 increases the level of T-cell activation in vivo, and this has been demonstrated to be beneficial as an adjunct treatment in tumor vaccine therapies. Since inhibition of CTLA4 signaling leads to very different outcomes than CD28 blockade during T-cell activation, it may be beneficial to design a CD80 and/or CD86 neutralizing therapeutic entity which inhibits the interaction of CD80 and/or CD86 with CD28 but not CTLA4, or vice versa.

CD80 and CD86 are also present at high levels on many lymphomas of B-cell origin. Thus, monoclonal antibodies, fragments thereof and other proteins binding CD80 and/or CD86 can be useful in the therapy of such tumors, either by recruiting effector functions, induction of cell death or as a targeting entity in immunotoxins or radiotoxin conjugates (Friedberg et al., Blood 106: 11 Abs 2435, 2005).

As both CD80 and CD86 bind to either counterreceptor, these molecules are thought to have at least partially overlapping functional roles (partial functional redundancy). It follows that, in order to achieve maximal therapeutic benefit, interactions of both CD80 and CD86 with either CD28 or CD152 need to be inhibited at the same time. Potentially, this can be achieved using soluble forms of CD152 (Abatacept, CTLA4-Ig, see Linsley et al. J. Exp. Med. 174: 561, 1991), affinity variants thereof (Belatacept, LEA29Y, see Larsen et al., Am. J. Transplant 5: 443, 2005) or CD28 (CD28-Ig, see Linsley et al., J. Exp. Med. 173: 721, 1991). No single monoclonal antibody has yet been described which can bind to both CD80 and CD86 (WO 04/076488, van den Beucken et al., J. Mol. Biol. 310: 591, 2001), although this would clearly be beneficial.

PD-1 is, similar to CD28, CTLA4 and ICOS, a transmembrane protein of the Ig superfamily. It shares 23% homology with CTLA4, but it lacks the motif required for B7-1 and B7-2 binding. PD-1 receptor is found on activated. T and B cells as well as myeloid cells such as macrophages. It binds two known ligands, Pd-L1 and PD-L2, found on professional APC, such as DC and monocytes, but also found constitutively on certain parencnhymal cells (in the heart, lung, and kidney) as well as on subpopulation of T and B cells (Freeman et al. 2000, J. Exp. Med. 192: 1027; Latchman et al. 2001, Nat. Immunol. 2: 261). In analgous manner to CTLA4, engagement of PD-1 by its ligands results in a negative regulatory effect, with inhibition of downstream cellular signaling events, diminished cellular proliferation, and cytokine production. PD-1 deficiency also results in autoimmune phenomena, including splenomegaly, B cell expansion with increased serum immunoglobulins, lupus-like glomerulonephritis, arthritis, and autoimmune cardiomyopathy (Nishimura et al. 1999, Immunity 11: 141).

Costimulatory pathways are e.g. further described in detail by Yamada et al. (2002, J. Am. Soc. Nephrol. 13: 559), by Coyle and Gutierrez-Ramos (2003, Nature Immunol. 2: 203) and by Coyle and Gutierrez-Ramos (2004, Springer Semin. Immun. 25: 349).

As illustrated above, in order for a cell surface molecule or receptor to trigger cell signaling and/or a certain downstream process, in certain cases more than one molecule or ligand binds to said cell surface molecule or receptor, or more than one cell surface molecule or receptor is activated by the binding of one or more binding molecules or ligands. Therefore, in order to obtain an efficient therapeutic intervention, all the different interactions that trigger the cell signaling and/or downstream process should be inhibited. This could be achieved by combining or mixing different binding molecules (Saito, Curr. Opin. Immunol. 10: 313, 1998; Lenschow, Science 257: 789, 1992), or by use of a bispecific or multispecific binding molecule (Dincq et al. Protein Expression and Purification 22: 11, 2001).

Proteins and peptides that bind to desired molecules are well known in the art. Some non-limiting examples include peptides and proteins with an immunoglobulin fold (i.e. immunoglobulins), such as antibodies and antibody fragments, binding units and binding molecules derived from antibodies and antibody fragments (such as heavy chain variables domains, light chain variable domains, domain antibodies and proteins and peptides suitable for use as domain antibodies, single domain antibodies and proteins and peptides suitable for use as single domain antibodies, Nanobodies® and dAb's; as well as suitable fragments of any of the foregoing), as well as constructs comprising such antibody fragments, binding units or binding molecules (such as scFv's and diabodies). Antibodies from mammals employ two immunoglobulin folds to recognize antigen. Small recombinant versions of such antibodies such as recombinant single-chain Fv or Fab fragments, have been used extensively to build bispecific or multispecific binding molecules, e.g. genetically fused scFv₂'s, diabodies, triabodies etc. (reviewed by Holliger and Hudson, Nature Biotechnology 23: 1126-36, 2005). Due the fact that they are composed of two non-covalently associated variable domains, bispecific or multispecific molecules based on scFv and Fab antibodies display many disadvantages including problems with expression and stability. The use of simpler antibodies, based on a single variable domain responsible for the interaction with antigen, has provided a simpler method for making bispecific or multispecific binding molecules. Naturally occurring single domain molecules based on the immunoglobulin V_{HH} domain from camelids are particularly suited for combining multiple binding sites into multivalent or multispecific molecules. These are naturally devoid of light chains and therefore don't have problems with expression, production and aggregation.

All these bispecific or multispecific binding molecules, however, still comprise or consist of two or more binding units (e.g. V_{HH} domains) that are linked. The linking of different binding sites (binding units) at different position in the binding molecule, however, increases, mostly even doubles the size of the therapeutic molecule and, consequently diminishes the advantage of small therapeutic molecules such as the ability to cross membranes and penetrate into physiological compartments, tissues and organs not accessible to other, larger therapeutic molecules. In addition, the fusion region itself may be sensitive to extra- or intracellular proteases. Finally the geometric orientation of the two binding sites in such molecule is influenced by multiple parameters including the linker length and composition as well as the precise binding epitopes on the target antigens. In some cases the orientation can be such that the binding affinity and kinetics of the two binding sites in said binding molecule are different from those of the original monovalent components.

Hence the creation of a bispecific or multispecific binding molecule by the linkage (e.g. via chemical or genetic fusion) of two independently isolated binding units, be it a peptide or other immunoglobulin domain, may for some applications have a number of disadvantages. In addition, smaller molecules are easier to handle, to produce and may have superior biophysical properties (such as solubility, stability).

The present inventors have now found that one binding molecule can have two binding sites, each for a different molecule (which may not share a high level of homology with each other) at partially or fully overlapping positions in the binding molecule (i.e. in one binding unit). As described above, the minimal size of such multispecific binding molecules could provide significant advantages in vivo. This provides a way to make small and compact therapeutic molecules, with significant potential as drug.

### SUMMARY OF THE INVENTION

The present invention relates to a multispecific binding molecule, “binder of the invention” or “multispecific binder of the invention” (such as dual specific, triple specific, quadruple specific, etc.) that contains at least two binding sites (“first and second antigen binding sites” or “first and second binding sites”), each directed against a different antigen or antigenic determinant. The first and second binding sites contained within said multispecific binder of the invention are positioned such that the binding site that interacts with the first antigen or antigenic determinant (“first antigen binding site” or “first binding site”) partially or fully overlaps in primary and/or tertiary structure with the binding site (“second antigen binding site” or “second binding site”) that interacts with the second antigen or antigenic determinant. In the “partially overlapping binding sites” at least 10%, 20%, 30%, 40%, preferably at least 50%, 60%, 70%, more preferably at least 80%, 85%, 90% or even 95% or more of the elements that form the primary and/or tertiary structure of the first antigen binding site are also elements that form the primary and/or tertiary structure of the second antigen binding site. In a preferred aspect of the invention, all elements that form the primary and/or tertiary structure of the first antigen binding site are also elements that form the primary and/or tertiary structure of the second antigen binding site (referred herein as “fully overlapping binding sites”).

The binding sites may be directed against any desired antigen or antigenic determinant, depending on the intended application or use of the multispecific binder of the invention. Such antigens and antigenic determinants will be clear to the skilled person, for instance based on the disclosure herein. In a preferred non-limiting aspect of the invention, the binding sites are directed against at least two naturally occurring binding molecules, such as against a first and second naturally occurring binding molecule. Accordingly, it is a specific object of the present invention to provide multispecific binders that are directed against (as defined herein) a first and a second naturally occurring binding molecule, in particular against a first and a second binding molecule naturally occurring in a warm-blooded animal, more in particular against a first and a second binding molecule naturally occurring in a mammal, and especially against a first and a second binding molecule naturally occurring in a human. Preferably said first and second naturally occurring binding molecules both interact with or bind to the same third naturally occurring binding molecule.

Said first and/or second naturally occurring binding molecule may be biological molecules present on the surface of at least one cell or tissue of a warm blooded animal, preferably a mammal, in particular a human, such as a receptor or ligand. They may be located on the same cell or on different cells. When they are located on different cells, they may for example be located in cells of a similar type or nature (e.g. on cells involved in the immune system/immune response) or of a different type or nature; and/or in cells that are part of the same tissue or organ or different tissues or organs.

In a preferred embodiment, said first and/or second naturally occurring binding molecules are located on the same cell. Said first and second naturally occurring binding molecule may belong to the same protein family or superfamily. In a preferred embodiment, said first and second naturally occurring binding molecules are PD-L1 and PD-L2 which belong to the B7 superfamily. Other examples of protein families and superfamilies that comprise such first and second naturally occurring binding molecules are known to the skilled person and are described further herein. Other examples of first, second and third naturally occurring binding molecules will also become clear from the further description herein.

When the first and second naturally occurring binding molecules are ligands, the third naturally occurring binding molecule may be a receptor. Else, the first and second naturally occurring binding molecules may be receptors and the third naturally occurring binding molecule may be a ligand. The multispecific binder of the present invention can generally be used to modulate, and in particular inhibit and/or prevent, binding of the at least two naturally occurring binding molecules to the third naturally occurring binding molecule, and thus to modulate, and in particular inhibit or prevent, the signalling that is mediated by the first, second and/or third naturally occurring binding molecule, to modulate the biological pathways in which the first, second and/or third naturally occurring binding molecules are involved, and/or to modulate the biological mechanisms, responses and effects associated with such signalling or these pathways. In a specific aspect of the invention, the multispecific binder of the invention antagonizes the signalling mediated by the first, second and/or third naturally occurring binding molecule. In another specific aspect of the invention, the multispecific binder agonizes the signalling mediated by the first, second and/or third naturally occurring binding molecule. In a preferred embodiment, the dual specific binder of the invention inhibits and/or blocks the interaction of PD-L1 and PD-L2 with. PD-1. Other examples of interactions that are inhibited and/or blocked by the multispecific binders of the invention will become clear from the further description herein.

As such, the multispecific binders of the present invention can be used for the prevention and/or treatment of certain diseases and/or disorders, which are characterized by excessive and/or unwanted signalling mediated by the first, second and/or third naturally occurring binding molecules or by the pathway(s) in which the first, second and/or third naturally occurring binding molecules are involved. Examples of such diseases and/or disorders will be clear to the skilled person based on the disclosure herein, and for example include the following diseases and disorders: cancer, inflammatory diseases, osteoporosis, melanoma, a tumor, soft tissue sarcoma, skin cancer, drug-resistant bony sarcomas, leukemia, Crohn's disease, rheumatoid arthritis, systemic lupus erythematosus, Sjogren's syndrome, lymphohistocytosis, myocarditis, multiple sclerosis, autoimmune encephalomeyeltitis, insulin-dependent diabetes mellitus, allergies, allograft rejection, xeno transplant rejection and/or grafi, versus host disease. Other applications and uses of the multispecific binders of the invention will become clear to the skilled person from the further disclosure herein.

Accordingly the present invention relates to the multispecific binders of the present invention for use as a medicament. In a preferred aspect, the present invention relates to the multispecific binders of the present invention for prevention and/or treatment of at least one cancer, inflammatory disease or osteoporosis and diseases as referred to above.

In the multispecific binders of the invention (as well as compounds comprising the same) the first and second antigen binding site allow said multispecific binder to bind to the at least two antigens or antigenic determinants to which said respective binding sites are directed, such as the first and second naturally occurring binding molecules, preferably with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In particular, the multispecific binders of the invention (as well as compounds comprising the same) are preferably such that they bind to each of the at least two antigens or antigenic determinants, such as the first and second naturally occurring binding molecules, with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles).

In a preferred, but non-limiting aspect of the invention, the multispecific binder of the invention (as well as a compound comprising the same) is such that it binds to the first naturally occurring binding molecule with a dissociation constant (K_{D}) that approximates the dissociation constant with which said multispecific binder (as well as a compound comprising the same) binds to the second naturally occurring binding molecule. Thus, by means of illustration and without limitation, when the multispecific binder of the invention (as well as a compound comprising the same) binds to the first naturally occurring binding molecule with a dissociation constant (K_{D}) of about 10⁻⁷ moles/liter and/or with a binding affinity (K_{A}) of about 10⁷ M⁻¹, said multispecific binder (as well as a compound comprising the same) also binds to the second naturally occurring binding molecule with a dissociation constant (K_{D}) of about 10⁻⁷ moles/liter and/or with a binding affinity (K_{A}) of about 10⁷ M⁻¹.

In another preferred, but non-limiting aspect of the invention, the multispecific binder of the invention (as well as a compound comprising the same) is such that it binds to the first naturally occurring binding molecule with a dissociation constant (K_{D}) that is at least 2 fold more, at least 5 fold more, at least 10 fold more, preferably at least 100 fold more, more preferably at least 1000 fold more, than the dissociation constant with which said multispecific binder (as well as a compound comprising the same) binds to the second naturally occurring binding molecule. Thus, by means of illustration and without limitation, when the multispecific binder of the invention (as well as a compound comprising the same) binds to the second naturally occurring binding molecule with a dissociation constant (K_{D}) of about 10⁻⁷ moles/liter and/or with a binding affinity (K_{A}) of about 10⁷ M⁻¹, said multispecific binder (as well as a compound comprising the same) may bind to the first naturally occurring binding molecule with a dissociation constant (K_{D}) of about 2·10⁻⁷ moles/liter, of about 5·10⁷ moles/liter, of about 10⁻⁶ moles/liter or more and/or with a binding affinity (K_{A}) of about 5·10⁶ M⁻¹, of about 2·10⁶ M⁻¹, of about 10⁶ M⁻¹ or less, preferably with a dissociation constant (K_{D}) of about 10⁻⁵ moles/liter or more and/or with a binding affinity (K_{A}) of about 10⁵ M⁻¹ or less, and more preferably with a dissociation constant (K_{D}) of about 10⁻⁴ moles/liter or more and/or with a binding affinity (K_{A}) of about 10⁴ M⁻¹ or less.

In another preferred, but non-limiting aspect of the invention, the multispecific binder of the invention (as well as a compound comprising the same) is such that it binds to the second naturally occurring binding molecule with a dissociation constant (K_{D}) that is at least 2 fold more, at least 5 fold more, at least 10 fold more, preferably at least 100 fold more, more preferably at least 1000 fold more, than the dissociation constant with which said multispecific binder (as well as a compound comprising the same) binds to the first naturally occurring binding molecule. Thus, by means of illustration and without limitation, when the multispecific binder of the invention (as well as a compound comprising the same) binds to the first naturally occurring binding molecule with a dissociation constant (K_{D}) of about 10⁻⁷ moles/liter and/or with a binding affinity (K_{A}) of about 10⁷ M⁻¹, said multispecific binder (as well as a compound comprising the same) may bind to the second naturally occurring binding molecule with a dissociation constant (K_{D}) of about 2·10⁻⁷ moles/liter, of about 5·10⁻⁷ moles/liter, of about 10⁻⁶ moles/liter or more and/or with a binding affinity (K_{A}) of about 5·10⁶ M⁻¹, of about 2·10⁶ M⁻¹, of about 10⁶ M⁻¹ or less, preferably with a dissociation constant (K_{D}) of about 10⁻⁵ moles/liter or more and/or with a binding affinity (K_{A}) of about 10⁵ M⁻¹ or less, and more preferably with a dissociation constant (K_{D}) of about 10⁴ moles/liter or more and/or with a binding affinity (K_{A}) of about 10⁴ M⁻¹ or less.

In another preferred, but non-limiting aspect of the invention, the multispecific binder of the invention (as well as a compound comprising the same) is such that it binds to the first naturally occurring binding molecule with a dissociation constant (K_{D}) that approximates (or that is lower or higher than) the dissociation constant with which the third naturally occurring binding molecule binds to said first naturally occurring binding molecule. Thus, by means of illustration and without limitation, when the third naturally occurring binding molecule binds to the first naturally occurring binding molecule with a dissociation constant (K_{D}) of about 10⁻⁷ moles/liter and/or with a binding affinity (K_{A}) of about 10⁷ M⁻¹, the multispecific binder of the invention (as well as a compound comprising the same) may also bind to said first naturally occurring binding molecule with a dissociation constant (K_{D}) of about 10⁻⁷ moles/liter (or lower or higher than 10⁻⁷ moles/liter) and/or with a binding affinity (K_{A}) of about 10⁷ M⁻¹ (or lower or higher than 10⁷ M⁻¹). Accordingly, in this preferred aspect of the invention, when the binder of the invention is directed against PD-L1 and PD-L2, said binder of the invention should bind to PD-L1 with a dissociation constant (K_{D}) that approximates (or that is lower or higher than) the dissociation constant with which PD-1 binds to PD-L1.

In another preferred, but non-limiting aspect of the invention, the multispecific binder of the invention (as well as a compound comprising the same) is such that it binds to the second naturally occurring binding molecule with a dissociation constant (K_{D}) that approximates (or that is lower or higher than) the dissociation constant with which the third naturally occurring binding molecule binds to said second naturally occurring binding molecule. Thus, by means of illustration and without limitation, when the third naturally occurring binding molecule binds to the second naturally occurring binding molecule with a dissociation constant (K_{D}) of about 10⁻⁷ moles/liter and/or with a binding affinity (K_{A}) of about 10⁷ M⁻¹, the multispecific binder of the invention (as well as a compound comprising the same) may also bind to said second naturally occurring binding molecule with a dissociation constant (K_{D}) of about 10⁻⁷ moles/liter (or lower or higher than 10⁻⁷ moles/liter) and/or with a binding affinity (K_{A}) of about 10⁷ M⁻¹ (or lower or higher than 10⁷ M⁻¹). Accordingly, in this preferred aspect of the invention, when the binder of the invention is directed against PD-L1 and PD-L2, said binder of the invention should bind to PD-L2 with a dissociation constant (K_{D}) that approximates (or that is lower or higher than) the dissociation constant with which PD-1 binds to PD-L2.

In another preferred, but non-limiting aspect of the invention, the multispecific binder of the invention (as well as a compound comprising the same) is such that it binds to the first naturally occurring binding molecule with a dissociation constant (K_{D}) that approximates (or that is lower or higher than) the dissociation constant with which the third naturally occurring binding molecule hinds to said first naturally occurring binding molecule and such that it binds to the second naturally occurring binding molecule with a dissociation constant (K_{D}) that approximates (or that is lower or higher than) the dissociation constant with which the third naturally occurring binding molecule binds to said second naturally occurring binding molecule. Thus, by means of illustration and without limitation, when the third naturally occurring binding molecule binds to the first and second naturally occurring binding molecules with a dissociation constant (K_{D}) of about 10⁻⁷ moles/liter and 10⁻⁶ moles/liter respectively and/or with a binding affinity (K_{A}) of about 10⁷ M⁻¹ and 10⁶ M⁻¹ respectively, the multispecific binder of the invention (as well as a compound comprising the same) may also bind to said first and second naturally occurring binding molecules with a dissociation constant (K_{D}) of about 10⁻⁷ moles/liter (or lower or higher than 10⁻⁷ moles/liter) and 10⁻⁶ moles/liter (or lower or higher than 10⁻⁶ moles/liter) respectively and/or with a binding affinity (K_{A}) of about 10⁷ M⁻¹ (or lower or higher than 10⁷ M⁻¹) and 10⁶ M⁻¹ (or lower or higher than 10⁶ M⁻¹) respectively. Accordingly, in this preferred aspect of the invention, when the binder of the invention is directed against PD-L1 and PD-L2, said binder of the invention should bind to PD-L1 and PD-L2 with a dissociation constant (K_{D}) that approximates (or that is lower or higher than) the dissociation constant with which PD-1 binds to PD-L1 and PD-L2 respectively.

The multispecific binders of the invention (as well as compounds comprising the same) are preferably also such that they bind to each of the at least two antigens or antigenic determinants, such as the first and second naturally occurring binding molecules, with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹.

In a preferred, but non-limiting aspect of the invention, the multispecific binder of the invention (as well as a compound comprising the same) is such that it binds to the first naturally occurring binding molecule with a kₒₙ-rate that approximates the kₒₙ-rate with which said multispecific binder (as well as a compound comprising the same) binds to the second naturally occurring binding molecule. Thus, by means of illustration and without limitation, when the multispecific binder of the invention (as well as a compound comprising the same) binds to the first naturally occurring binding molecule with a kₐ-rate of about 10⁴ M⁻¹s⁻¹, said multispecific binder (as well as a compound comprising the same) also binds to the second naturally occurring binding molecule with a kₒₙ-rate of about 10⁴

In another preferred, but non-limiting aspect of the invention, the multispecific binder of the invention (as well as a compound comprising the same) is such that it binds to the first naturally occurring binding molecule with a kₒₙ-rate that is at least 2 fold more at least 5 fold more, at least 10 fold more, preferably at least 100 fold more, more preferably at least 1000 fold more, than the kₒₙ-rate with which said multispecific binder of the invention (as well as a compound comprising the same) binds to the second naturally occurring binding molecule. Thus, by means of illustration and without limitation, when the multispecific binder of the invention (as well as a compound comprising the same) binds to the second naturally occurring binding molecule with a kₒₙ-rate of about 10⁴M⁻¹s⁻¹, said multispecific binder (as well as a compound comprising the same) may bind to the first naturally occurring binding molecule with a kₒₙ-rate of about 2·10⁴ M⁻¹s⁻¹, of about 5·10⁴ M⁻¹s⁻¹, of about 10⁵M⁻¹s⁻¹ or more, preferably with a kₒₙ-rate of about 10⁶ M⁻¹s⁻¹ or more, and more preferably with a kₒₙ-rate of about 10⁷ M⁻¹s⁻¹ or more.

In another preferred, but non-limiting aspect of the invention, the multispecific binder of the invention (as well as a compound comprising the same) is such that it binds to the second naturally occurring binding molecule with a kₒₙ-rate that is at least 2 fold more, at least 5 fold more, at least 10 fold more, preferably at least 100 fold more, more preferably at least 1000 fold more, than the kₒₙ-rate with which said multispecific binder (as well as a compound comprising the same) binds to the first naturally occurring binding molecule. Thus, by means of illustration and without limitation, when the multispecific binder of the invention (as well as a compound comprising the same) binds to the first naturally occurring binding molecule with a kₒₙ-rate of about 10⁴ M⁻¹s⁻¹, said multispecific binder (as well as a compound comprising the same) may bind to the second naturally occurring binding molecule with a kₒₙ-rate of about 2·10⁴ of about 5·10⁴ M⁻¹s⁻¹, of about 10⁵ M⁻¹s⁻¹ or more, preferably with a kₒₙ-rate of about 10⁶ M⁻¹s⁻¹ or more, and more preferably with a kₒₙ-rate of about 10⁷ M⁻¹s⁻¹ or more.

In another preferred, but non-limiting aspect of the invention, the multispecific binder of the invention (as well as a compound comprising the same) is such that it binds to the first naturally occurring binding molecule with a kₒₙ-rate that approximates (or that is lower or higher than) the kₒₙ-rate with which the third naturally occurring binding molecule binds to said first naturally occurring binding molecule. Thus, by means of illustration and without limitation, when the third naturally occurring binding molecule binds to the first naturally occurring binding molecule with a kₒₙ-rate of about 10⁴ M⁻¹s⁻¹, the multispecific binder of the invention (as well as a compound comprising the same) may also bind to said first naturally occurring binding molecule with a kₒₙ-rate of about 10⁴ M⁻¹s⁻¹ (or lower or higher than 10⁴ M⁻¹s⁻¹). Accordingly, in this preferred aspect of the invention, when the binder of the invention is directed against PD-L1 and PD-L2, said binder of the invention should bind to PD-L1 with a kₒₙ-rate that approximates (or that is lower or higher than) the kₒₙ-rate with which PD-1 binds to PD-L1.

In another preferred, but non-limiting aspect of the invention, the multispecific binder of the invention (as well as a compound comprising the same) is such that it binds to the second naturally occurring binding molecule with a kₒₙ-rate that approximates (or that is lower or higher than) the kₒₙ-rate with which the third naturally occurring binding molecule binds to said second naturally occurring binding molecule. Thus, by means of illustration and without limitation, when the third naturally occurring binding molecule binds to the second naturally occurring binding molecule with a kₒₙ-rate of about 10⁴ M⁻¹s⁻¹, the multispecific binder of the invention (as well as a compound comprising the same) may also bind to said second naturally occurring binding molecule with a kₒₙ-rate of about 10⁴ M⁻¹s⁻¹ (or lower or higher than 10⁴ M⁻¹s⁻¹). Accordingly, in this preferred aspect of the invention, when the binder of the invention is directed against PD-L1 and PD-L2 said binder of the invention should bind to PD-L2 with a kₒₙ-rate that approximates (or that is lower or higher than) the kₒₙ-rate with which PD-1 binds to PD-L2.

In another preferred, but non-limiting aspect of the invention, the multispecific binder of the invention (as well as a compound comprising the same) is such that it binds to the first naturally occurring binding molecule with a kₒₙ-rate that approximates (or that is lower or higher than) the kₒₙ-rate with which the third naturally occurring binding molecule binds to said first naturally occurring binding molecule and such that it binds to the second naturally occurring binding molecule with a kₒₙ-rate that approximates (or that is lower or higher than) the kₒₙ-rate with which the third naturally occurring binding molecule binds to said second naturally occurring binding molecule. Thus, by means of illustration and without limitation, when the third naturally occurring binding molecule binds to the first and second naturally occurring binding molecules with a kₒₙ-rate of about 10⁴ M⁻¹s⁻¹ and 10⁵ M⁻¹s⁻¹ respectively, the multispecific binder of the invention (as well as a compound comprising the same) may also bind to said first and second naturally occurring binding molecules with a kₒₙ-rate of about 10⁴M⁻¹s⁻¹ (or lower or higher than 10⁴ M⁻¹s⁻¹) and 10⁵ M⁻¹s⁻¹ (or lower or higher than 10⁵ M⁻¹s⁻¹) respectively. Accordingly, in this preferred aspect of the invention, when the binder of the invention is directed against PD-L1 and PD-L2, said binder of the invention should bind to PD-L1 and PD-L2 with a kₒₙ-rate that approximates (or that is lower or higher than) the kₒₙ-rate with which PD-1 binds to PD-L1 and PD-L2 respectively.

The multispecific binders of the invention (as well as compounds comprising the same) are preferably also such that they bind to each of the at least two antigens or antigenic determinants, such as the first and second naturally occurring binding molecules, with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s¹.

In a preferred, but non-limiting aspect of the invention, the multispecific binder of the invention (as well as a compound comprising the same) is such that it binds to the first naturally occurring binding molecule with a k_{off}-rate that approximates the k_{off}-rate with which said multispecific binder (as well as a compound comprising the same) binds to the second naturally occurring binding molecule. Thus, by means of illustration and without limitation, when the multispecific binder of the invention (as well as a compound comprising the same) binds to the first naturally occurring binding molecule with a k_{off}-rate of about 10⁻⁴ s⁻¹, said multispecific binder (as well as a compound comprising the same) also binds to the second naturally occurring binding molecule with a k_{off}-rate of about 10⁻⁴ s⁻¹.

In another preferred, but non-limiting aspect of the invention, the multispecific binder of the invention (as well as a compound comprising the same) is such that it binds to the first naturally occurring binding molecule with a k_{off}-rate that is at least 2 fold more, at least 5 fold more, at least 10 fold more, preferably at least 100 fold more, more preferably at least 1000 fold more, than the k_{off}-rate with which said multispecific binder (as well as a compound comprising the same) binds to the second naturally occurring binding molecule. Thus, by means of illustration and without limitation, when the multispecific binder of the invention (as well as a compound comprising the same) binds to the second naturally occurring binding molecule with a k_{off}-rate of about 10⁻⁴ s⁻¹, said multispecific binder (as well as a compound comprising the same) may bind to the first naturally occurring binding molecule with a k_{off}-rate of about 2. 10⁻⁴ s⁻¹, of about 5·10⁻⁴ s⁻¹, of about 10⁻³ s⁻¹ or more, preferably with a k_{off}-rate of about 10⁻² s⁻¹ or more, and more preferably with a k_{off}-rate of about 10⁻¹ s⁻¹ or more.

In another preferred, but non-limiting aspect of the invention, the multispecific binder of the invention (as well as compounds comprising the same) is such that it binds to the second naturally occurring binding molecule with a k_{off}-rate that is at least 2 fold more, at least 5 fold more, at least 10 fold more, preferably at least 100 fold more, more preferably at least 1000 fold more, than the k_{off}-rate with which said multispecific binder (as well as a compound comprising the same) binds to the first naturally occurring binding molecule. Thus, by means of illustration and without limitation, when the multispecific binder of the invention (as well as a compound comprising the same) binds to the first naturally occurring binding molecule with a k_{off}-rate of about 10⁻⁴ s⁻¹, said multispecific binder (as well as a compound comprising the same) may bind to the second naturally occurring binding molecule with a k_{off}-rate of about 2. 10⁻⁴ s⁻¹, of about 5·10⁻⁴ s⁻¹, of about 10⁻³ s⁻¹ or more, preferably with a k_{off}-rate of about 10⁻² s⁻¹ or more, and more preferably with a k_{off}-rate of about 10⁻¹ s⁻¹ or more.

In another preferred, but non-limiting aspect of the invention, the multispecific binder of the invention (as well as a compound comprising the same) is such that it binds to the first naturally occurring binding molecule with a k_{off}-rate that approximates (or that is lower or higher than) the k_{off}-rate with which the third naturally occurring binding molecule binds to said first naturally occurring binding molecule. Thus, by means of illustration and without limitation, when the third naturally occurring binding molecule binds to the first naturally occurring binding molecule with a k_{off}-rate of about 10⁻⁴ s⁻¹, the multispecific binder of the invention (as well as a compound comprising the same) may also hind to said first naturally occurring binding molecule with a k_{off}-rate of about 10⁻⁴ s⁻¹ (or lower or higher than 10⁻⁴ s⁻¹). Accordingly, in this preferred aspect of the invention, when the binder of the invention is directed against PD-L1 and PD-L2, said binder of the invention should bind to PD-L1 with a k_{off}-rate that approximates (or that is lower or higher than) the k_{off}-rate with which PD-1 binds to PD-L1.

In another preferred, but non-limiting aspect of the invention, the multispecific binder of the invention (as well as a compound comprising the same) is such that it binds to the second naturally occurring binding molecule with a k_{off}-rate that approximates (or that is lower or higher than) the a k_{off}-rate with which the third naturally occurring binding molecule binds to said second naturally occurring binding molecule. Thus, by means of illustration and without limitation, when the third naturally occurring binding molecule binds to the second naturally occurring binding molecule with a k_{off}-rate of about 10⁻⁴ s⁻¹, the multispecific binder of the invention (as well as a compound comprising the same) may also bind to said second naturally occurring binding molecule with a k_{off}-rate of about 10⁻⁴ s⁻¹ (or lower or higher than 10⁻⁴ s⁻¹). Accordingly, in this preferred aspect of the invention, when the binder of the invention is directed against PD-L1 and PD-L2, said binder of the invention should bind to PD-L2 with a k_{off}-rate that approximates (or that is lower or higher than) the k_{off}-rate with which PD-1 binds to PD-L2.

In another preferred, but non-limiting aspect of the invention, the multispecific binder of the invention (as well as a compound comprising the same) is such that it binds to the first naturally occurring binding molecule with a k_{off}-rate that approximates (or that is lower or higher than) the k_{off}-rate with which the third naturally occurring binding molecule binds to said first naturally occurring binding molecule and such that it binds to the second naturally occurring binding molecule with a k_{off}-rate that approximates (or that is lower or higher than) the k_{off}-rate with which the third naturally occurring binding molecule binds to said second naturally occurring binding molecule. Thus, by means of illustration and without limitation, when the third naturally occurring binding molecule binds to the first and second naturally occurring binding molecule with a k_{off}-rate of about 10⁻⁴ s⁻¹ and 10⁻³ s⁻¹ respectively, the multispecific binder of the invention (as well as a compound comprising the same) may also bind to said first and second naturally occurring binding molecule with a k_{off}-rate of about 10⁻⁴ s⁻¹ (or lower or higher than 10⁻⁴ s⁻¹) and 10⁻³ s⁻¹ (or lower or higher than 10⁻³ s⁻¹) respectively. Accordingly, in this preferred aspect of the invention, when the binder of the invention is directed against PD-L1 and PD-L2, said binder of the invention should bind to PD-L1 and PD-L2 with a k_{off}-rate that approximates (or that is lower or higher than) the k_{off}-rate with which PD-1 binds to PD-L1 and PD-L2 respectively.

The multispecific binder of the invention (as well as a compound comprising the same) is preferably such that it will bind to each of the at least two antigens or antigenic determinants, such as the first and second naturally occurring binding molecules, with an affinity less than 500 nM, preferably less than 200 nM, more preferably less than 10 nM, such as less than 500 pM.

Some preferred IC₅₀ values for binding of multispecific binders of the invention (as well as compounds comprising the same) to each of the at least two antigens or antigenic determinants, such as the first and second naturally occurring binding molecules, will become clear from the further description and examples herein.

The multispecific binder of the invention may be any molecule (or a derivative thereof, such as a pegylated derivative) that can bind to (as described herein) and/or has affinity for at least two antigens or antigenic determinants such as the first and second naturally occurring binding molecule. They are preferably in essentially isolated form (as defined herein), or form part of a compound of the invention (as defined herein), which may comprise or essentially consist of one or more binders of the invention and which may optionally further comprise one or more further molecules or amino acid sequences (all optionally linked via one or more suitable linkers). For example, and without limitation, the one or more binder of the invention may be used as a binding unit in such a compound, which may optionally contain one or more further molecules or amino acid sequences that can serve as a binding unit (i.e. against one or more other targets than the first and second naturally occurring binding molecule, so as to provide a monovalent or multivalent compound of the invention, respectively, all as described herein). Such a compound may also be in essentially isolated form (as defined herein).

Generally, when a binder of the invention (or a compound or polypeptide comprising the same) is intended for administration to a subject (for example for therapeutic and/or diagnostic purposes as described herein), it is preferably either a binder that does not occur naturally in said subject; or, when it does occur naturally in said subject, in essentially isolated form (as defined herein).

The binder of the invention can be any binding molecule known per se. Examples of binding molecules will be clear from the description herein.

In one specific, but non-limiting aspect, the binder of the invention may be an amino acid sequence (also referred herein as “amino acid sequence of the invention”) and in particular a polypeptide or protein with an immunoglobulin fold or may be an amino acid sequence that, under suitable conditions (such as physiological conditions) is capable of forming an immunoglobulin fold (i.e. by folding). Reference is inter alia made to the review by Halaby et al., J. Protein Eng. 12, 563-71 (1999). Preferably, when properly folded so as to form an immunoglobulin fold, such an amino acid sequence is capable of specific binding (as defined herein) to the first and second naturally occurring binding molecule; and more preferably capable of binding to the first and second naturally occurring binding molecule with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Also, parts, fragments, analogs, mutants, variants, alleles and/or derivatives of such amino acid sequences are preferably such that they comprise an immunoglobulin fold or are capable for forming, under suitable conditions, an immunoglobulin fold.

Also, according to one specific, but non-limiting aspect, the amino acid sequence of the invention may comprise or essentially consist of four framework regions (FR1 to FR4 respectively) separated from each other by three complementarity determining regions (CDR1 to CDR3 respectively); or any suitable parts, fragments, analogs, homologs, orthologs, variants, derivatives, etc. of such amino acid sequence. As further described herein, such parts or fragments preferably at least comprise at least one CDR of such an amino acid sequence. For example, an amino acid sequence of the invention may be chosen from the group consisting of antibodies and antibody fragments, binding units and binding molecules derived from antibodies or antibody fragments, and antibody fragments, binding units or binding molecules. In particular, such amino acid sequence of the invention may be an immunoglobulin variable domain sequence or a suitable fragment thereof, such as light chain variable domain sequence (e.g. a V_{L}-sequence) or a suitable fragment thereof; or a heavy chain variable domain sequence (e.g. a V_{H}-sequence) or a suitable fragment thereof. When the amino acid sequence of the invention is a heavy chain variable domain sequence, it may be a heavy chain variable domain sequence that is derived from a conventional four-chain antibody (such as, without limitation, a V_{H} sequence that is derived from a human antibody) or be a so-called V_{HH}-sequence (as defined herein) that is derived from a so-called “heavy chain antibody” (as defined herein).

However, it should be noted that the invention is not limited as to the origin of the amino acid sequence of the invention (or of the nucleotide sequence of the invention used to express it), nor as to the way that the amino acid sequence or nucleotide sequence of the invention is (or has been) generated or obtained. Thus, the amino acid sequences of the invention may be naturally occurring amino acid sequences (from any suitable species) or synthetic or semi-synthetic amino acid sequences. In a specific but non-limiting aspect of the invention, the amino acid sequence is a naturally occurring immunoglobulin sequence (from any suitable species) or a synthetic or semi-synthetic immunoglobulin sequence, including but not limited to “humanized” (as defined herein) immunoglobulin sequences (such as partially or fully humanized mouse or rabbit immunoglobulin sequences, and in particular partially or fully humanized V_{HH} sequences or Nanobodies), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Reference is for example made to the standard handbooks, as well as to the further description and prior art mentioned herein.

The amino acid sequence of the invention may in particular be a domain antibody (or an amino acid sequence that is suitable for use as a domain antibody), a single domain antibody (or an amino acid sequence that is suitable for use as a single domain antibody), a “dAb” (or an amino acid sequence that is suitable for use as a dAb) or a Nanobody® (as defined herein, and including but not limited to a V_{HH} sequence); or any suitable fragment of any one thereof.

In particular, the amino acid sequence of the invention may be a Nanobody® (as defined herein) or a suitable fragment thereof. [Note: Nanobody®, Nanobodies® and Nanoclone® are registered trademarks of Ahlynx N. V.] Such Nanobodies directed against a first and second naturally occurring binding molecule will also be referred to herein as “Nanobodies of the invention”. Again, such Nanobodies may be derived in any suitable manner and from any suitable source, and may for example be naturally occurring V_{HH} sequences (i.e. from a suitable species of Camelid) or synthetic or semi-synthetic amino acid sequences, including but not limited to “humanized” (as defined herein) Nanobodies, “camelized” (as defined herein) immunoglobulin sequences (and in particular camelized heavy chain variable domain sequences), as well as Nanobodies that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein. Also, when a Nanobody comprises a V_{HH} sequence, said Nanobody may be suitably humanized, as further described herein, so as to provide one or more further (partially or fully) humanized Nanobodies of the invention. Similarly, when a Nanobody comprises a synthetic or semi-synthetic sequence (such as a partially humanized sequence), said Nanobody may optionally be further suitably humanized, again as described herein, again so as to provide one or more further (partially or fully) humanized Nanobodies of the invention.

Depending on how the amino acid sequence of the invention is chosen, it preferably comprises between 4 and 500 amino acid residues, more preferably between 5 and 300 amino acid residues, and even more preferably between 10 and 200 amino acid residues, such as between 20 and 150 amino acid residues, for example about 30, 40, 50, 60, 70, 80, 90, 100, 110, 120, 130 or 140 amino acid residues.

As discussed above, for binding to its at least two antigens or antigenic determinants, such as the at least two naturally occurring binding molecules, the multispecific binder of the invention contains at least two partially or fully overlapping binding sites. Accordingly, the amino acid sequence of the invention will contain within its amino acid sequence at least two partially or fully overlapping binding sites or stretches of amino acid residues via which the amino acid sequence of the invention can bind to its at least two antigens or antigenic determinants such as the at least two naturally occurring binding molecules. These at least two partially or fully overlapping stretches of amino acid residues thus each form the “site” (also referred to herein as the “antigen binding site”) for binding to one of the at least two naturally occurring binding molecules.

In the partially overlapping stretches of amino acid residues, at least 10%, 20%, 30%, 40%, preferably at least 50%, 60%, 70%, more preferably at least 80%, 85%, 90% or even 95% or more of the amino acid residues that form the primary and/or tertiary structure of the first antigen binding site are also the amino acid residues that form the primary and/or tertiary structure of the second antigen binding site. In one aspect of the invention, at least 10%, 20%, 30%, 40%, preferably at least 50%, 60%, 70%, more preferably at least 80%, 85%, 90% or even 95% or more of the amino acid residues that form the primary structure of the first antigen binding site are also the amino acid residues that form the primary structure of the second antigen binding site. In another aspect of the invention, at least 10%, 20%, 30%, 40%, preferably at least 50%, 60%, 70%, more preferably at least 80%, 85%, 90% or even 95% or more of the amino acid residues that form the tertiary structure of the first antigen binding site are also the amino acid residues that form the tertiary structure of the second antigen binding site.

In a preferred aspect of the invention, all amino acid residues that form the primary and/or tertiary structure of the first antigen binding site are also the amino acid residues that form the primary and/or tertiary structure of the second antigen binding site (referred herein as “fully overlapping binding sites”). In one preferred aspect of the invention, all amino acid residues that form the primary structure of the first antigen binding site are also the amino acid residues that form the primary structure of the second antigen binding site. In another preferred aspect of the invention, all amino acid residues that form the tertiary structure of the first antigen binding site are also the amino acid residues that form the tertiary structure of the second antigen binding site.

Thus, by means of illustration and without limitation, in binding sites that partially overlap in primary structure, when the primary structure of the first antigen binding site consists of 10 amino acid residues, at least 1, 2, 3, 4, preferably at least 5, 6, 7, more preferably at least 8, 9 of these amino acid residues should also form the primary structure of the second antigen binding site; in binding sites that fully overlap in primary structure, when the primary structure of the first antigen binding site consists of 10 amino acid residues, all 10 of these amino acid residues should also form the primary structure of the second antigen binding site; in binding sites that partially overlap in tertiary structure, when the tertiary structure of the first antigen binding site consists of 5 amino acid residues, at least 1, 2, preferably at least 3, more preferably at least 4 of these amino acid residues should also form the tertiary structure of the second antigen binding site; or in binding sites that fully overlap in tertiary structure, when the tertiary structure of the first antigen binding site consists of 5 amino acid residues, all 5 of these amino acid residues should also form the tertiary structure of the second antigen binding site.

Generally, in this context, the amino acid sequence of the invention may be any amino acid sequence that comprises at least two partially or fully overlapping stretches of amino acid residues, in which each stretch of amino acid residues has an antigen binding site (i.e. wherein the stretch of amino acids that interacts with the first naturally occurring binding molecule partially or fully overlaps in primary and/or tertiary structure with the stretch of amino acids that interacts with the second naturally occurring binding molecule). Such an amino acid sequence may or may not comprise an immunoglobulin fold. In one specific, non-limiting embodiment, the amino acid sequences of the invention are small linear peptides that essentially do not comprise an immunoglobulin fold. In this embodiment the amino acid sequences of the invention may comprise between 3 and 50, preferably between 5 and 40, such as about 10, 15, 20 or 25 amino acid residues as long as they comprise at least two partially or fully overlapping stretches of amino acid residues, in which each stretch of amino acid residues has an antigen binding site, as defined above. Such peptides may for example be small synthetic or semi-synthetic peptides. In another specific non-limiting embodiment, such an amino acid sequence may be a suitable fragment of an immunoglobulin sequence as long as it comprises at least two partially or fully overlapping stretches of amino acid residues, in which each stretch of amino acid residues has an antigen binding site, as defined above.

These partially or fully overlapping stretches of amino acid residues may be derived from or comprise at least one CDR from an immunoglobulin that is directed against the first and second naturally occurring binding molecule (i.e. in which said immunoglobulin may be as described herein). For example, such partially or fully overlapping stretches of amino acid residues may be derived from or comprise at least one CDR (such as CDR1, CDR2, and in particular CDR3) from a heavy chain variable domain, light chain variable domain, domain antibodies, single domain antibodies, Nanobodies® or dAb's and in particular from a Nanobody of the invention. Reference is for example made to WO 03/050531 (Ablynx N.V. and Algonomics Nev.), which describes methods for the identification and selection of peptides, in particular immunoglobulin heavy chain variable domain CDR sequences that bind to a given target or targets of interest. Alternatively, such an amino acid sequence may be a suitable “protein scaffold” that comprises at least two partially or fully overlapping stretches of amino acid residues that corresponds to at least one CDR sequence (or part thereof). Suitable scaffolds for presenting amino acid sequences will be clear to the skilled person, and for example comprise, without limitation, to binding scaffolds based on or derived from immunoglobulins (i.e. other than the immunoglobulin sequences already described herein), protein scaffolds derived from protein A domains (such as Affibodies™), tendamistat, fibronectin, lipocalin, CTLA-4, T-cell receptors, designed ankyrin repeats, avimers and PDZ domains (Binz et al., Nat. Biotech. 23: 1257, 2005), and binding moieties based on DNA or RNA including but not limited to DNA or RNA aptamers (Ulrich et al., Comb. Chem. High Throughput Screen 9: 619-32, 2006).

In this context, the invention provides a number of CDR sequences (i.e. small peptides) that are particularly suited for binding to PD-L1 and PD-L2. Each of these CDR sequences may form (part of) the at least two partially or fully overlapping antigen binding sites or stretches of amino acid residues of the amino acid sequence of the invention. It should be noted that the invention in its broadest sense is not limited to a specific structural role or function that these CDR sequences may have in an amino acid sequence of the invention, as long as these CDR sequences allow the amino acid sequence of the invention to bind to PD-L1 and PD-L2. Thus, the invention provides an amino acid sequence that is capable of binding to PD-L1 and PD-L2 and that comprises one or more CDR sequences as described herein, and in particular a suitable combination of two or more such CDR sequences, that are suitably linked to each other via one or more further amino acid sequences, such that the entire amino acid sequence forms two partially or fully overlapping binding sites that are capable of binding to PD-L1 and PD-L2. It should however also be noted that the presence of only one such CDR sequence in the amino acid sequence of the invention may by itself already be sufficient to provide an amino acid sequence of the invention that is capable of binding to PD-L1 and PD-L2.

Thus, in one specific, but non-limiting aspect, the amino acid sequence of the invention may be an amino acid sequence that comprises at least one amino acid sequence that is chosen from the group consisting of the CDR1 sequences, CDR2 sequences and CDR3 sequences that are described herein (or any suitable combination thereof). Thus, the amino acid sequence of the invention may be an amino acid sequence that comprises at least two partially or fully overlapping antigen binding sites, wherein said partially or fully overlapping antigen binding sites comprise at least one amino acid sequence that is chosen from the group consisting of the CDR1 sequences, CDR2 sequences and CDR3 sequences that are described herein (or any suitable combination thereof).

Again, any amino acid sequence of the invention that comprises one or more of these CDR sequences is preferably such that it can specifically bind (as defined herein) to PD-L1 and PD-L2, and more in particular such that it can bind to PD-L1 and PD-L2 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein), that is as defined herein.

More in particular, the amino acid sequences of the invention may be any amino acid sequence that comprises at least two partially or fully overlapping antigen binding sites, wherein said partially or fully overlapping antigen binding sites comprise at least two amino acid sequences that are chosen from the group consisting of the CDR1 sequences described herein, the CDR2 sequences described herein and the CDR3 sequences described herein, such that (i) when the first amino acid sequence is chosen from the CDR1 sequences described herein, the second amino acid sequence is chosen from the CDR2 sequences described herein or the CDR3 sequences described herein; (ii) when the first amino acid sequence is chosen from the CDR2 sequences described herein, the second amino acid sequence is chosen from the CDR1 sequences described herein or the CDR3 sequences described herein; or (iii) when the first amino acid sequence is chosen from the CDR3 sequences described herein, the second amino acid sequence is chosen from the CDR1 sequences described herein or the CDR2 sequences described herein.

Even more in particular, the amino acid sequences of the invention may be amino acid sequences that comprise at least two partially or fully overlapping antigen binding sites, wherein said partially or fully overlapping antigen binding sites comprise at least three amino acid sequences that are chosen from the group consisting of the CDR1 sequences described herein, the CDR2 sequences described herein and the CDR3 sequences described herein, such that the first amino acid sequence is chosen from the CDR1 sequences described herein, the second amino acid sequence is chosen from the CDR2 sequences described herein, and the third amino acid sequence is chosen from the CDR3 sequences described herein. Preferred combinations of CDR1, CDR2 and CDR3 sequences will become clear from the further description herein. As will be clear to the skilled person, such an amino acid sequence is preferably an immunoglobulin sequence (as further described herein), but it may for example also be any other amino acid sequence that comprises a suitable scaffold for presenting said CDR sequences.

Thus, in one specific, but non-limiting aspect, the invention relates to an amino acid sequence directed against PD-L1 and PD-L2 that comprises one or more CDR sequences chosen from the group consisting of:
a) the amino acid sequence of SEQ ID NO's: 4-6;
b) amino acid sequences that have at least 80% amino acid identity with the amino acid sequence of SEQ ID NO's: 4-6;
c) amino acid sequences that have 3, 2, or 1 amino acid difference with the amino acid sequence of SEQ ID NO's: 4-6;
d) the amino acid sequences of SEQ ID NO's: 10-12;
e) amino acid sequences that have at least 80% amino acid identity with at least one of the amino acid sequences of SEQ ID NO's: 10-12;
f) amino acid sequences that have 3, 2, or 1 amino acid difference with at least one of the amino acid sequences of SEQ ID NO's: 10-12;
g) the amino acid sequences of SEQ ID NO's: 16-18;
h) amino acid sequences that have at least 80% amino acid identity with at least one of the amino acid sequences of SEQ ID NO's: 16-18;
i) amino acid sequences that have 3, 2, or 1 amino acid difference with at least one of the amino acid sequences of SEQ ID NO's: 16-18;
   or any suitable combination thereof.

When an amino acid sequence of the invention contains one or more amino acid sequences according to b) and/or c):
i) any amino acid substitution in such an amino acid sequence according to b) and/or c) is preferably, and compared to the corresponding amino acid sequence according to a), a conservative amino acid substitution, (as defined herein);
   and/or
ii) the amino acid sequence according to b) and/or c) preferably only contains amino acid substitutions, and no amino acid deletions or insertions, compared to the corresponding amino acid sequence according to a);
   and/or
iii) the amino acid sequence according to b) and/or c) may be an amino acid sequence that is derived from an amino acid sequence according to a) by means of affinity maturation using one or more techniques of affinity maturation known per se. Similarly, when an amino acid sequence of the invention contains one or more
   amino acid sequences according to e) and/or f):
i) any amino acid substitution in such an amino acid sequence according to e) and/or f) is preferably, and compared to the corresponding amino acid sequence according to d), a conservative amino acid substitution, (as defined herein);
   and/or
ii) the amino acid sequence according to e) and/or f) preferably only contains amino acid substitutions, and no amino acid deletions or insertions, compared to the corresponding amino acid sequence according to d);
   and/or
iii) the amino acid sequence according to e) and/or f) may be an amino acid sequence that is derived from an amino acid sequence according to d) by means of affinity maturation using one or more techniques of affinity maturation known per se.

Also, similarly, when an amino acid sequence of the invention contains one or more amino acid sequences according to h) and/or i):
i) any amino acid substitution in such an amino acid sequence according to h) and/or i) is preferably, and compared to the corresponding amino acid sequence according to g), a conservative amino acid substitution, (as defined herein);
   and/or
ii) the amino acid sequence according to h) and/or i) preferably only contains amino acid substitutions, and no amino acid deletions or insertions, compared to the corresponding amino acid sequence according to g);
   and/or
iii) the amino acid sequence according to h) and/or i) may be an amino acid sequence that is derived from an amino acid sequence according to g) by means of affinity maturation using one or more techniques of affinity maturation known per se.

It should be understood that the last preceding paragraphs also generally apply to any amino acid sequences of the invention that comprise one or more amino acid sequences according to b), c), e), f), h) or i), respectively.

In this specific aspect, the amino acid sequence preferably comprises one or more CDR sequences chosen from the group consisting of:
i) the amino acid sequence of SEQ ID NO's: 4-6;
ii) the amino acid sequences of SEQ ID NO's: 10-12; and
iii) the amino acid sequences of SEQ ID NO's: 16-18;
   or any suitable combination thereof.

Also, preferably, in such an amino acid sequence, the at least one of said CDR sequences forms part of the antigen binding sites for binding against PD-L1 and PD-L2.

In a more specific, but again non-limiting aspect, the invention relates to an amino acid sequence directed against PD-L1 and PD-L2 that comprises two or more CDR sequences chosen from the group consisting of:
a) the amino acid sequence of SEQ ID NO's: 4-6;
b) amino acid sequences that have at least 80% amino acid identity with the amino acid sequence of SEQ ID NO's: 4-6;
c) amino acid sequences that have 3, 2, or 1 amino acid difference with the amino acid sequence of SEQ ID NO's: 4-6;
d) the amino acid sequences of SEQ ID NO's: 10-12;
e) amino acid sequences that have at least 80% amino acid identity with at least one of the amino acid sequences of SEQ ID NO's: 10-12;
f) amino acid sequences that have 3, 2, or 1 amino acid difference with at least one of the amino acid sequences of SEQ ID NO's: 10-12;
g) the amino acid sequences of SEQ ID NO's: 16-18;
h) amino acid sequences that have at least 80% amino acid identity with at least one of the amino acid sequences of SEQ ID NO's: 16-18;
i) amino acid sequences that have 3, 2, or 1 amino acid difference with at least one of the amino acid sequences of SEQ ID NO's: 16-18;
   such that (i) when the first CDR sequence corresponds to one of the amino acid sequences according to a), b) or c), the second CDR sequence corresponds to one of the amino acid sequences according to d), e), f), g), h) or i); (ii) when the first CDR sequence corresponds to one of the amino acid sequences according to d), e) or f), the second CDR sequence corresponds to one of the amino acid sequences according to a), b), c), g), h) or i); or (iii) when the first CDR sequence corresponds to one of the amino acid sequences according to g), h) or i), the second CDR sequence corresponds to one of the amino acid sequences according to a), b), c), d), e) or f).

In this specific aspect, the amino acid sequence preferably comprises two or more CDR sequences chosen from the group consisting of:
i) the amino acid sequence of SEQ ID NO's: 4-6;
ii) the amino acid sequences of SEQ ID NO's: 10-12; and
iii) the amino acid sequences of SEQ ID NO's: 16-18;
   such that, (i) when the first CDR sequence corresponds to the amino acid sequence of SEQ ID NO's: 4-6, the second CDR sequence corresponds to one of the amino acid sequences of SEQ ID NO's: 10-12 or of SEQ ID NO's: 16-18; (ii) when the first CDR sequence corresponds to one of the amino acid sequences of SEQ ID NO's: 10-12, the second CDR sequence corresponds to the amino acid sequence of SEQ ID NO's: 4-6 or of SEQ ID NO's: 16-18; or (iii) when the first CDR sequence corresponds to one of the amino acid sequences of SEQ ID NO's: 16-18, the second CDR sequence corresponds to the amino acid sequence of SEQ ID NO's: 4-6 or of SEQ ID NO's: 10-12.

Also, in such an amino acid sequence, the at least two CDR sequences again preferably form part of the antigen binding sites for binding against PD-L1 and PD-L2.

In an even more specific, but non-limiting aspect, the invention relates to an amino acid sequence directed against PD-L1 and PD-L2, that comprises three or more CDR sequences, in which the first CDR sequence is chosen from the group consisting of:
a) the amino acid sequence of SEQ ID NO's: 4-6;
b) amino acid sequences that have at least 80% amino acid identity with the amino acid sequence of SEQ ID NO's: 4-6;
c) amino acid sequences that have 3, 2, or 1 amino acid difference with the amino acid sequence of SEQ ID NO's: 4-6;
   the second CDR sequence is chosen from the group consisting of:
d) the amino acid sequences of SEQ ID NO's: 10-12;
e) amino acid sequences that have at least 80% amino acid identity with at least one of the amino acid sequences of SEQ ID NO's: 10-12;
f) amino acid sequences that have 3, 2, or 1 amino acid difference with at least one of the amino acid sequences of SEQ ID NO's: 10-12;
   and the third CDR sequence is chosen from the group consisting of:
g) the amino acid sequences of SEQ ID NO's: 16-18;
h) amino acid sequences that have at least 80% amino acid identity with at least one of the amino acid sequences of SEQ ID NO's: 16-18;
i) amino acid sequences that have 3, 2, or 1 amino acid difference with at least one of the amino acid sequences of SEQ ID NO's: 16-18.

Preferably, in this specific aspect, the first CDR sequence is chosen from the group consisting of the amino acid sequence of SEQ ID NO's: 4-6; the second CDR sequence is chosen from the group consisting of the amino acid sequences of SEQ ID NO's: 10-12; and the third CDR sequence is chosen from the group consisting of the amino acid sequences of SEQ ID NO's: 16-18.

Again, preferably, in such an amino acid sequence, the at least three CDR sequences form part of the antigen binding sites for binding against PD-L1 and PD-L2.

Preferred combinations of such CDR sequences will become clear from the further disclosure herein.

Preferably, in such amino acid sequences the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 22-24. This degree of amino acid identity can for example be determined by determining the degree of amino acid identity (in a manner described herein) between said amino acid sequence and one or more of the sequences of SEQ ID NO's: 22-24, in which the amino acid residues that form the framework regions are disregarded. Also, such amino acid sequences of the invention can be as further described herein.

Also, such amino acid sequences are preferably such that they can specifically bind (as defined herein) to PD-L1 and PD-L2; and more in particular bind to PD-L1 and PD-L2 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

When the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequence of SEQ ID NO's: 4-6;
b) amino acid sequences that have at least 80% amino acid identity with the amino acid sequence of SEQ ID NO's: 4-6;
c) amino acid sequences that have 3, 2, or 1 amino acid difference with the amino acid sequences of SEQ ID NO's: 4-6;
   and/or

CDR2 is chosen from the group consisting of:
d) the amino acid sequences of SEQ ID NO's: 10-12;
e) amino acid sequences that have at least 80% amino acid identity with at least one of the amino acid sequences of SEQ ID NO's: 10-12;
f) amino acid sequences that have 3, 2, or 1 amino acid difference with at least one of the amino acid sequences of SEQ ID NO's: 10-12;
   and/or

CDR3 is chosen from the group consisting of:
g) the amino acid sequences of SEQ ID NO's: 16-18;
h) amino acid sequences that have at least 80% amino acid identity with at least one of the amino acid sequences of SEQ ID NO's: 16-18;
i) amino acid sequences that have 3, 2, or 1 amino acid difference with at least one of the amino acid sequences of SEQ ID NO's: 16-18.

In particular, such an amino acid sequence of the invention may be such that CDR1 is chosen from the group consisting of the amino acid sequence of SEQ ID NO's: 4-6; and/or CDR2 is chosen from the group consisting of the amino acid sequences of SEQ ID NO's: 10-12; and/or CDR3 is chosen from the group consisting of the amino acid sequences of SEQ ID NO's: 16-18.

In particular, when the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequence of SEQ ID NO's: 4-6;
b) amino acid sequences that have at least 80% amino acid identity with the amino acid sequence of SEQ ID NO's: 4-6;
c) amino acid sequences that have 3, 2, or 1 amino acid difference with the amino acid sequence of SEQ ID NO's: 4-6;
   and

CDR2 is chosen from the group consisting of:
d) the amino acid sequences of SEQ ID NO's: 10-12;
e) amino acid sequences that have at least 80% amino acid identity with at least one of the amino acid sequences of SEQ ID NO's: 10-12;
f) amino acid sequences that have 3, 2, or 1 amino acid difference with at least one of the amino acid sequences of SEQ ID NO's: 10-12;
   and

CDR3 is chosen from the group consisting of:
g) the amino acid sequences of SEQ ID NO's: 16-18;
h) amino acid sequences that have at least 80% amino acid identity with at least one of the amino acid sequences of SEQ ID NO's: 16-18;
i) amino acid sequences that have 3, 2, or 1 amino acid difference with at least one of the amino acid sequences of SEQ ID NO's: 16-18; or any suitable fragment of such an amino acid sequence

In particular, such an amino acid sequence of the invention may be such that CDR1 is chosen from the group consisting of the amino acid sequence of SEQ ID NO's: 4-6; and CDR2 is chosen from the group consisting of the amino acid sequences of SEQ ID NO's: 10-12; and CDR3 is chosen from the group consisting of the amino acid sequences of SEQ ID NO's: 16-18.

Again, preferred combinations of CDR sequences will become clear from the further description herein.

Also, such amino acid sequences are preferably such that they can specifically bind (as defined herein) to PD-L1 and PD-L2; and more in particular bind to PD-L1 and PD-L2 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In one preferred, but non-limiting aspect, the invention relates to an amino acid sequence that essentially consists of 4 framework regions (FR1 to FR4, respectively) and 3 complementarity determining regions (CDR1 to CDR3, respectively), in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 22-24. This degree of amino acid identity can for example be determined by determining the degree of amino acid identity (in a manner described herein) between said amino acid sequence and one or more of the sequences of SEQ ID NO's: 22-24, in which the amino acid residues that form the framework regions are disregarded. Such amino acid sequences of the invention can be as further described herein.

In such an amino acid sequence of the invention, the framework sequences may be any suitable framework sequences. Examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

The framework sequences are preferably such that the amino acid sequence of the invention is a domain antibody (or an amino acid sequence that is suitable for use as a domain antibody); is a single domain antibody (or an amino acid sequence that is suitable for use as a single domain antibody); is a “dAb” (or an amino acid sequence that is suitable for use as a dAb); or is a Nanobody® (including but not limited to V_{HH} sequence). Again, suitable framework sequences will be clear to the skilled person, for example on the basis the standard handbooks and the further disclosure and prior art mentioned herein.

In particular, the framework sequences present in the amino acid sequences of the invention may contain one or more of Hallmark residues (as defined herein), such that the amino acid sequence of the invention is a Nanobody®. Some preferred, but non-limiting examples of (suitable combinations of) such framework sequences will become clear from the further disclosure herein.

In another preferred, but non-limiting aspect, the invention relates to a Nanobody with an amino acid sequence that is chosen from the group consisting of SEQ ID NO's: 22-24 or from the group consisting of from amino acid sequences that have more than 80%, preferably more than 90%, more preferably more than 95%, such as 99% or more sequence identity (as defined herein) with at least one of the amino acid sequences of SEQ ID NO's: 22-24.

Again, as generally described herein for the amino acid sequences of the invention, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived). Such fragments may also again be such that they comprise or can form an immunoglobulin fold, or alternatively be such that they do not comprise or cannot form an immunoglobulin fold.

In one specific aspect, such a fragment comprises a single CDR sequence as described herein (and in particular a CDR3 sequence), that is flanked on each side by (part of) a framework sequence (and in particular, part of the framework sequence(s) that, in the immunoglobulin sequence from which the fragment is derived. are adjacent to said CDR sequence. For example, a CDR3 sequence may be preceded by (part of) a FR3 sequence and followed by (part of) a FR4 sequence). Such a fragment may also contain a disulphide bridge, and in particular a disulphide bridge that links the two framework regions that precede and follow the CDR sequence, respectively (for the purpose of forming such a disulphide bridge, cysteine residues that naturally occur in said framework regions may be used, or alternatively cysteine residues may be synthetically added to or introduced into said framework regions). For a further description of these “Expedite fragments”, reference is again made to WO 03/050531, as well as to the US provisional application of Ablynx N.V. entitled “Peptides capable of binding to serum proteins” of Ablynx N.V. (inventors: Revets, Hilde Adi Pierrette; Kolkman, Joost Alexander; and Hoogenboom, Hendricus Renerus Jacobus Mattheus) filed on Dec. 5, 2006 (see also PCT/EP2007/063348).

In another aspect, the invention also relates to a compound or construct, and in particular a protein or polypeptide (also referred to herein as a “compound of the invention” or “polypeptide of the invention”, respectively) that comprises or essentially consists of one or more multispecific binders or amino acid sequences of the invention (or suitable fragments thereof), and optionally further comprises one or more other groups, residues, moieties or binding units. As will become clear to the skilled person from the further disclosure herein, such further groups, residues, moieties, binding units or amino acid sequences may or may not provide further functionality to the binder or amino acid sequence of the invention (and/or to the compound or construct in which it is present) and may or may not modify the properties of the binder or amino acid sequence of the invention.

For example, such further groups, residues, moieties or binding units may be one or more additional amino acid sequences, such that the compound or construct is a (fusion.) protein or (fusion) polypeptide. In a preferred but non-limiting aspect, said one or more other groups, residues, moieties or binding units are immunoglobulin sequences. Even more preferably, said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”s, amino acid sequences that are suitable for use as a dAb, or Nanobodies.

Alternatively, such groups, residues, moieties or binding units may for example be chemical groups, residues, moieties, which may or may not by themselves be biologically and/or pharmacologically active. For example, and without limitation, such groups may be linked to the one or more amino acid sequences of the invention so as to provide a “derivative” of an amino acid sequence or polypeptide of the invention, as further described herein.

Also within the scope of the present invention are compounds or polypeptides, that comprises or essentially consists of one or more derivatives as described herein, and optionally further comprises one or more other groups, residues, moieties or binding units, optionally linked via one or more linkers. Preferably, said one or more other groups, residues, moieties or binding units are amino acid sequences.

In the compounds or polypeptides described above, the one or more binders or amino acid sequences of the invention and the one or more groups, residues, moieties or binding units may be linked directly to each other and/or via one or more suitable linkers or spacers. For example, when the binder and the one or more groups, residues, moieties or binding units are amino acid sequences, the linkers may also be amino acid sequences, so that the resulting compound is a fusion (protein) or fusion (polypeptide).

The compounds or polypeptides of the invention can generally be prepared by a method which comprises at least one step of suitably linking the one or more binders or amino acid sequences of the invention to the one or more further groups, residues, moieties or binding units, optionally via the one or more suitable linkers, so as to provide the compound or polypeptide of the invention. Polypeptides of the invention can also be prepared by a method which generally comprises at least the steps of providing a nucleic acid that encodes a polypeptide of the invention, expressing said nucleic acid in a suitable manner, and recovering the expressed polypeptide of the invention. Such methods can be performed in a manner known per se, which will be clear to the skilled person, for example on the basis of the methods and techniques further described herein.

The process of designing/selecting and/or preparing a compound or polypeptide of the invention, starting from a binder or amino acid sequence of the invention, is also referred to herein as “formatting” said binder or amino acid sequence of the invention; and a binder or an amino acid of the invention that is made part of a compound or polypeptide of the invention is said to be “formatted” or to be “in the format of” said compound or polypeptide of the invention. Examples of ways in which a binder or an amino acid sequence of the invention can be formatted and examples of such formats will be clear to the skilled person based on the disclosure herein; and such formatted binders or amino acid sequences form a further aspect of the invention.

In one specific aspect of the invention, a compound of the invention or a polypeptide of the invention may have an increased half-life, compared to the corresponding binder or amino acid sequence of the invention as further described herein. Some preferred, but non-limiting examples of such compounds and polypeptides will become clear to the skilled person based on the further disclosure herein, and for example comprise binders or amino acid sequences of the invention that have been chemically modified to increase the half-life thereof (for example, by means of pegylation); binders or amino acid sequences of the invention that comprise at least one additional binding site for binding to a serum protein (such as serum albumin); or compounds or polypeptides of the invention that comprise at least one binder or amino acid sequence of the invention that is linked to at least one moiety (and in particular at least one amino acid sequence) that increases the half-life of the binder or amino acid sequence of the invention. Examples of compounds or polypeptides of the invention that comprise such half-life extending moieties or amino acid sequences will become clear to the skilled person based on the further disclosure herein; and for example include, without limitation, compounds or polypeptides in which the one or more binder or amino acid sequences of the invention are suitable linked to one or more serum proteins or fragments thereof (such as (human) serum albumin or suitable fragments thereof) or to one or more binding units that can bind to serum proteins (such as, for example, domain antibodies, amino acid sequences that are suitable for use as a domain antibody, single domain antibodies, amino acid sequences that are suitable for use as a single domain antibody, “dAb”s, amino acid sequences that are suitable for use as a dAb, or Nanobodies that can bind to serum proteins such as serum albumin (such as human serum albumin), serum immunoglobulins such as IgG, or transferrine; reference is made to the further description and references mentioned herein); compounds or polypeptides in which a binder or amino acid sequence of the invention is linked to an Fc portion (such as a human Fe) or a suitable part or fragment thereof; or compounds or polypeptides in which the one or more binders or amino acid sequences of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled. “Peptides capable of binding to serum proteins” of Ablynx N.V. filed on Dec. 5, 2006 (see also PCT/EP2007/063348).

It is also within the scope of the invention to use parts, fragments, analogs, mutants, variants, alleles and/or derivatives of the binders or amino acid sequences of the invention, and/or to use compounds or polypeptides comprising or essentially consisting of one or more of such parts, fragments, analogs, mutants, variants, alleles and/or derivatives, as long as these are suitable for the uses envisaged herein. Such parts, fragments, analogs, mutants, variants, alleles and/or derivatives will usually contain (at least part of) at least two partially or fully overlapping functional antigen-binding sites for binding to the first and a second naturally occurring binding molecules; and more preferably will be capable of specifically binding to said first and second naturally occurring binding molecules, and even more preferably capable of binding to said first and second naturally occurring binding molecules with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Some non-limiting examples of such parts, fragments, analogs, mutants, variants, alleles, derivatives, compounds and/or polypeptides will become clear from the further description herein. Additional fragments, compounds or polypeptides of the invention may also be provided by suitably combining (i.e. by linking or genetic fusion) one or more (smaller) parts or fragments as described herein.

In another aspect, the invention relates to a nucleic acid that encodes an amino acid sequence of the invention or a polypeptide of the invention (or a suitable fragment thereof). Such a nucleic acid will also be referred to herein as a “nucleic acid of the invention” and may for example be in the form of a genetic construct, as further described herein.

The nucleotide sequences of the invention may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

The invention further includes genetic constructs that include the foregoing nucleotide sequences or nucleic acids and one or more elements for genetic constructs known per se. The genetic construct may be in the form of a plasmid or vector. Such and other genetic constructs are known by those skilled in the art and will be further described herein.

In another aspect, the invention relates to a host or host cell that expresses (or that under suitable circumstances is capable of expressing) an amino acid sequence of the invention and/or a polypeptide of the invention; and/or that contains a nucleic acid or genetic construct of the invention. Some preferred but non-limiting examples of such hosts or host cells will become clear from the further description herein.

The invention further relates to a composition containing or comprising at least one binder of the invention, at least one amino acid sequence of the invention, at least one compound of the invention, at least one polypeptide of the invention (or a suitable fragment thereof) and/or at least one nucleic acid of the invention, and optionally one or more further components of such compositions known per se, i.e. depending on the intended use of the composition. Such a composition may for example be a pharmaceutical composition (as described herein), a veterinary composition or a composition for diagnostic use (as also described herein). Some preferred but non-limiting examples of such compositions will become clear from the further description herein.

The invention further relates to methods for generating and/or preparing the binders, amino acid sequences, Nanobodies, compounds, polypeptides, nucleic acids, host cells, and compositions described herein. Some preferred but non-limiting examples of such methods will become clear from the further description herein.

The invention further relates to applications and uses of the binders, amino acid sequences, Nanobodies, compounds, polypeptides, nucleic acids, host cells, products and compositions described herein, as well as to methods for the prevention and/or treatment of diseases and disorders associated with the first, second and/or third naturally occurring binding molecules. Some preferred but non-limiting applications and uses will become clear from the further description herein.

Generally, it is an object of the invention to provide pharmacologically active agents, as well as compositions comprising the same, that can be used in the diagnosis, prevention and/or treatment of certain diseases and disorders and to provide methods for the diagnosis, prevention and/or treatment of such diseases and disorders that involve the administration and/or use of such agents and compositions.

In particular, it is an object of the invention to provide such pharmacologically active agents, compositions and/or methods that have certain advantages compared to the agents, compositions and/or methods based on the use of a combination of different monospecific binding molecules or based on the use of multispecific binding molecules with more than one binding unit (i.e. in which the antigen binding sites are not partially or fully overlapping). These advantages will become clear from the further description below.

In particular, it is a specific object of the present invention to provide such binders and such compounds and/or polypeptides that are suitable for prophylactic, therapeutic and/or diagnostic use, in particular in a warm-blooded animal, and more particular in a mammal, and even more particular in a human being.

More in particular, it is a specific object of the present invention to provide binders and such compounds and/or polypeptides that can be used for the prevention, treatment, alleviation and/or diagnosis of one or more diseases, disorders or conditions associated with the first, second and/or third naturally occurring binding molecule and/or mediated by said first, second and/or third naturally occurring binding molecule (such as the diseases, disorders and conditions mentioned herein), in particular in a warm-blooded animal, more particular in a mammal, and even more particular in a human being.

It is also a specific object of the invention to provide such binders and such compounds and/or polypeptides that can be used in the preparation of pharmaceutical or veterinary compositions for the prevention and/or treatment of one or more diseases, disorders or conditions associated with and/or mediated by said first, second and/or third naturally occurring binding molecule (such as the diseases, disorders and conditions mentioned herein), in particular in a warm-blooded animal, more particular in a mammal, and even more in particular in a human being.

In the invention, generally, these objects are achieved by the use of the binders, compounds, polypeptides and compositions that are described herein.

Other aspects, embodiments, advantages and applications of the invention will also become clear from the further description herein, in which the invention will be described and discussed in more detail with reference to the Nanobodies of the invention and polypeptides of the invention comprising the same, which form some of the preferred aspects of the invention.

As will become clear from the further description herein, Nanobodies generally offer certain advantages (outlined herein) compared to “dAb's” or similar (single) domain antibodies or immunoglobulin sequences, which advantages are also provided by the Nanobodies of the invention. However, it will be clear to the skilled person that the more general aspects of the teaching herein can also be applied (either directly or analogously) to other binders and amino acid sequences of the invention.

### DETAILED DESCRIPTION

In the present description, examples and claims:
a) Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd.Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, New York (2005), as well as to the general background art cited herein;
b) Unless indicated otherwise, the term “immunoglobulin sequence”—whether used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acid sequences or nucleotide sequences encoding the same, unless the context requires a more limited interpretation; Also, the term “nucleotide sequence” as used herein also encompasses a nucleic acid molecule with said nucleotide sequence, so that the terms “nucleotide sequence” and “nucleic acid” should be considered equivalent and are used interchangeably herein;
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2(1): 49-57; Irving et al., J. Immunol. Methods, 2001, 248(1-2), 31-45; Schmitz et al., Placenta, 2000, 21 Suppl. A, S106-12, Gonzales et al., Tumour Biol., 2005, 26(1), 31-43, which describe techniques for protein engineering, such as affinity maturation and other techniques for improving the specificity and other desired properties of proteins such as immunoglobulins.
d) Amino acid residues will be indicated according to the standard three-letter or one-letter amino acid code, as mentioned in. Table A-1;

TABLE A-1
one-letter and three-letter amino acid code

| | | | |
|---|---|---|---|
| Nonpolar, | Alanine | Ala | A |
| uncharged | Valine | Val | V |
| (at pH 6.0-7.0)⁽³⁾ | Leucine | Leu | L |
| | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |

Notes:
⁽¹⁾Sometimes also considered to be a polar uncharged amino acid.
⁽²⁾Sometimes also considered to be a nonpolar uncharged amino acid.
⁽³⁾As will be clear to the skilled person, the fact that an amino acid residue is referred to in this Table as being either charged or uncharged at pH 6.0 to 7.0 does not reflect in any way on the charge said amino acid residue may have at a pH lower than 6.0 and/or at a pH higher than 7.0; the amino acid residues mentioned in the Table can be either charged and/or uncharged at such a higher or lower pH, as will be clear to the skilled person.
⁽⁴⁾As is known in the art, the charge of a His residu is greatly dependant upon even small shifts in pH, but a His residue can generally be considered essentially uncharged at a pH of about 6.5.

e) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).
   Alternatively, the degree of sequence identity between two or more nucleotide sequences may be calculated using a known computer algorithm for sequence alignment such as NCBI Blast v2.0, using standard settings.
   Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.
   Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;
f) For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein.
   Alternatively, the degree of sequence identity between two amino acid sequences may be calculated using a known computer algorithm, such as those mentioned above for determining the degree of sequence identity for nucleotide sequences, again using standard settings.
   Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.
   Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.
   Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (h) polar, negatively charged residues and their (uncharged) amides: Asp, Asn, Glu and Gln; (c) polar, positively charged residues: His, Arg and Lys; (d) large aliphatic, nonpolar residues: Met, Leu, Be, Val and Cys; and (e) aromatic residues: Phe, Tyr and Trp.
   Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nad. Acad. Sci. USA 81: 140-144, 1984; Kyte & Doolittle; J. Molec. Biol. 157: 105-132, 1981, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional domains form the V_{H}/V_{L} interface and potential camelizing substitutions on these positions can be found in the prior art cited above.
g) Amino acid sequences and nucleic acid sequences are said to be “exactly the same” if they have 100% sequence identity (as defined herein) over their entire length;
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
i) A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis;
j) When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the firstmentioned nucleotide sequence or amino acid sequence, respectively, but more usually this generally means that the firstmentioned nucleotide sequence or amino acid sequence comprises within its sequence a stretch of nucleotides or amino acid residues, respectively, that has the same nucleotide sequence or amino acid sequence, respectively, as the latter sequence, irrespective of how the firstmentioned sequence has actually been generated or obtained (which may for example be by any suitable method described herein). By means of a non-limiting example, when a Nanobody of the invention is said to comprise a CDR sequence, this may mean that said CDR sequence has been incorporated into the Nanobody of the invention, but more usually this generally means that the Nanobody of the invention contains within its sequence a stretch of amino acid residues with the same amino acid sequence as said CDR sequence, irrespective of how said Nanobody of the invention has been generated or obtained. It should also be noted that when the latter amino acid sequence has a specific biological or structural function, it preferably has essentially the same, a similar or an equivalent biological or structural function in the firstmentioned amino acid sequence (in other words, the firstmentioned amino acid sequence is preferably such that the latter sequence is capable of performing essentially the same, a similar or an equivalent biological or structural function). For example, when a Nanobody of the invention is said to comprise a CDR sequence or framework sequence, respectively, the CDR sequence and framework are preferably capable, in said Nanobody, of functioning as a CDR sequence or framework sequence, respectively. Also, when a nucleotide sequence is said to comprise another nucleotide sequence, the firstmentioned nucleotide sequence is preferably such that, when it is expressed into an expression product (e.g. a polypeptide), the amino acid sequence encoded by the latter nucleotide sequence forms part of said expression product (in other words, that the latter nucleotide sequence is in the same reading frame as the firstmentioned, larger nucleotide sequence).
k) The term “domain” as used herein generally refers to a globular region of an amino acid sequence (such as an antibody chain, and in particular to a globular region of a heavy chain antibody), or to a polypeptide that essentially consists of such a globular region. Usually, such a domain will comprise peptide loops (for example 3 or 4 peptide loops) stabilized, for example, as a sheet or by disulfide bonds. The term “binding domain” refers to such a domain that is directed against an antigenic determinant (as defined herein);
l) The term “antigenic determinant” refers to the epitope on the antigen or target recognized by the antigen-binding molecule (such as the binder, amino acid or Nanobody of the invention) and more in particular by the antigen-binding site of said molecule. The terms “antigenic determinant” and “epitope” may also be used interchangeably herein.
m) An amino acid sequence (such as a Nanobody, an antibody, a polypeptide of the invention, or generally an antigen binding protein or polypeptide or a fragment thereof) that can (specifically) bind to, that (specifically) recognizes, that interacts with, that has affinity for and/or that has specificity for a specific antigenic determinant, epitope, antigen or protein (or for at least one part, fragment or epitope thereof) is said to be “against”, “directed against” or “directed to” said antigenic determinant, epitope, antigen or protein.
n) The specific elements, parts or amino acid residues of an amino acid sequence (such as a Nanobody, an antibody, a polypeptide of the invention, or generally an antigen binding protein or polypeptide or a fragment thereof) with which said amino acid sequence interacts with a specific antigenic determinant, epitope, antigen or protein (or for at least one part, fragment or epitope thereof) is said to be the “antigen binding site”, “binding site” or “binding domain” of said amino acid sequence.
o) The term “specificity” refers to the number of different types of antigens or antigenic determinants to which a particular antigen-binding molecule (such as the multispecific binder or compound of the invention) can bind. The specificity of an antigen-binding molecule can be determined based on affinity and/or avidity. The affinity, represented by the equilibrium constant for the dissociation of an antigen with an antigen-binding molecule (K_{D}), is a measure for the binding strength between an antigenic determinant and an antigen-binding site on the antigen-binding molecule: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as the multispecific binder or compound of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the binders, the amino acid sequences, the Nanobodies, the compounds and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (ETA) and sandwich competition assays, and the different variants thereof known per se in the art; as well as the other techniques mentioned herein. The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}]. The affinity denotes the strength or stability of a molecular interaction. The affinity is commonly given as by the K_{D}, or dissociation constant, which has units of mol/liter (or M). The affinity can also be expressed as an association constant, K_{A}, which equals 1/K_{D} and has units of (mol/liter)⁻¹ (or M⁻¹). In the present specification, the stability of the interaction between two molecules (such as a binder, an amino acid sequence, a Nanobody, a compound or polypeptide of the invention and its intended target) will mainly be expressed in terms of the K_{D} value of their interaction; it being clear to the skilled person that in view of the relation K_{A}=1/K_{D}, specifying the strength of molecular interaction by its K_{D} value can also be used to calculate the corresponding K_{A} value. The K_{D}-value characterizes the strength of a molecular interaction also in a thermodynamic sense as it is related to the free energy (DG) of binding by the well known relation DG=RT.ln(K_{D}) (equivalently DG=−RT.ln(K_{A})), where R equals the gas constant, T equals the absolute temperature and In denotes the natural logarithm.
   The K_{D} for biological interactions which are considered meaningful (e.g. specific) are typically in the range of 10⁻¹⁰M (0.1 nM) to 10⁻⁵M (10000 nM). The stronger an interaction is, the lower is its K_{D}.
   The K_{D} can also be expressed as the ratio of the dissociation rate constant of a complex, denoted as k_{off}, to the rate of its association, denoted kₒ, (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹ s⁻¹. The on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=1n(2)/k_{off}. The off-rate may vary between 10⁻⁶ s⁻¹ (near irreversible complex with a t_{1/2} of multiple days) to 1 s⁻¹ (t_{1/2}=0.69 s).
   The affinity of a molecular interaction between two molecules can be measured via different techniques known per se, such as the well known surface plasmon resonance (SPR) biosensor technique (see for example Ober et al., Intern. Immunology, 13, 1551-1559, 2001) where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIACORE instruments. It will also be clear to the skilled person that the measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule. In such situation the measured affinity may be affected by the avidity of the interaction by the two molecules.
   Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artefacts relating to adsorption of one of the molecules on a support such as plastic.
   However, the accurate measurement of K_{D} may be quite labor-intensive and as consequence, often apparent K_{D} values are determined to assess the binding strength of two molecules. It should be noted that as long all measurements are made in a consistent way (e.g. keeping the assay conditions unchanged) apparent K_{D} measurements can be used as an approximation of the true K_{D} and hence in the present document K_{D} and apparent K_{D} should be treated with equal importance or relevance.
   Finally, it should be noted that in many situations the experienced scientist may judge it to be convenient to determine the binding affinity relative to some reference molecule. It is further a preferred aspect of the invention to provide binders that have affinity (K_{D}, kₒₙ-rate, and/or k_{off}-rate) for a first naturally occurring binding molecule that approximates, is more, or is less than its affinity for a second naturally occurring binding molecule. Therefore, to assess the binding strength between, for example, molecules A and B, one may e.g. use a reference molecule C that is known to bind to B and that is suitably labelled with a fluorophore or chromophore group or other chemical moiety, such as biotin for easy detection in an ELISA or FACS (Fluorescent activated cell sorting) or other format (the fluorophore for fluorescence detection, the chromophore for light absorption detection, the biotin for streptavidin-mediated ELISA detection). Typically, the reference molecule C is kept at a fixed concentration and the concentration of A is varied for a given concentration or amount of B. As a result an IC₅₀ value is obtained corresponding to the concentration of A at which the signal measured for C in absence of A is halved. Provided K_{D ref}, the K_{D} of the reference molecule, is known, as well as the total concentration c_{ref} of the reference molecule, the apparent K_{D} for the interaction A-B can be obtained from following formula: K_{D}=IC₅₀/(1+c_{ref}/K_{D ref}). Note that if c_{ref}<<K_{D ref}, K_{D}≈IC₅₀. Provided the measurement of the IC₅₀ is performed in a consistent way (e.g. keeping c_{ref} fixed) for the binders that are compared, the strength or stability of a molecular interaction can be assessed by the IC₅₀ and this measurement is judged as equivalent to K_{D} or to apparent K_{D} throughout this text.
p) The half-life of a binder, amino acid sequence, compound or polypeptide of the invention can generally be defined as the time taken for the serum concentration of the binder, amino acid sequence, compound or polypeptide to be reduced by 50%, in vivo, for example due to degradation of the sequence or compound and/or clearance or sequestration of the sequence or compound by natural mechanisms. The in vivo half-life of a binder, amino acid sequence, compound or polypeptide of the invention can be determined in any manner known per se, such as by pharmacokinetic analysis. Suitable techniques will be clear to the person skilled in the art, and may for example generally involve the steps of suitably administering to a warm-blooded animal (i.e. to a human or to another suitable mammal, such as a mouse, rabbit, rat, pig, dog or a primate, for example monkeys from the genus Macaca (such as, and in particular, cynomologus monkeys (Macaca fascicularis) and/or rhesus monkeys (Macaca mulatta)) and baboon (Papio ursinus)) a suitable dose of the binder, amino acid sequence, compound or polypeptide of the invention; collecting blood samples or other samples from said animal; determining the level or concentration of the binder, amino acid sequence, compound or polypeptide of the invention in said blood sample; and calculating, from (a plot of) the data thus obtained, the time until the level or concentration of the binder, amino acid sequence, compound or polypeptide of the invention has been reduced by 50% compared to the initial level upon dosing. Reference is for example made to the Experimental. Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982).
   As will also be clear to the skilled person (see for example pages 6 and 7 of WO 04/003019 and in the further references cited therein), the half-life can be expressed using parameters such as the t1/2-alpha, t1/2-beta and the area under the curve (AUC). In the present specification, an “increase in half-life” refers to an increase in any one of these parameters, such as any two of these parameters, or essentially all three these parameters. As used herein “increase in half-life” or “increased half-life”in particular refers to an increase in the t1/2-beta, either with or without an increase in the t1/2-alpha and/or the AUC or both.
q) The Figures, Sequence Listing and the Experimental Part/Examples are only given to further illustrate the invention and should not be interpreted or construed as limiting the scope of the invention and/or of the appended claims in any way, unless explicitly indicated otherwise herein.

In the present invention, multispecific binding molecules or multispecific binders are provided that have the capacity of binding more than one, i.e. at least two, antigens or antigenic determinants. “Multispecificity” as used for the binders of the present invention refers to their binding to two or more (structurally) different antigens or antigenic determinants. This multispecificity is achieved by the at least two partially or fully overlapping binding sites or antigen binding sites (“first and second antigen binding sites”) on the binders of the invention. Each of these at least two partially or fully overlapping binding sites comprises or consists of two or more elements that are adjacent to each other or that are in close proximity to each other and that form respectively the primary and tertiary structure of the antigen binding site. The at least two antigen binding sites on the binders of the invention partially or fully overlap with their primary structure and/or they may partially or fully overlap with their tertiary structure. When the antigen binding sites partially overlap with their primary structure, part of the elements that form the primary structure of the first antigen binding site are also the elements that form the primary structure of the second antigen binding site. When the antigen binding sites fully overlap with their primary structure, all of the elements that form the primary structure of the first antigen binding site are also the elements that form the primary structure of the second antigen binding site. When the antigen binding sites partially overlap with their tertiary structure, part of the elements that form the tertiary structure of the first antigen binding site are also the elements that form the tertiary structure of the second antigen binding site. When the antigen binding sites fully overlap with their tertiary structure, all of the elements that form the tertiary structure of the first antigen binding site are also the elements that form the tertiary structure of the second antigen binding site.

In the partially overlapping binding sites at least 10%, 20%, 30%, 40%, preferably at least 50%, 60%, 70%, more preferably at least 80%, 85%, 90% or even 95% or more of the elements that form the primary and/or tertiary structure of the first antigen binding site are also the elements that form respectively the primary and/or tertiary structure of the second antigen binding site. In fully overlapping binding sites, all elements that form the primary and/or tertiary structure of the first antigen binding site are the elements that form respectively the primary and/or tertiary structure of the second antigen binding site.

In the amino acids of the invention, each binding site essentially consists of a stretch of amino acid residues that comprises or consists of two or more amino acid residues that are adjacent to each other or that are in close proximity to each other and that form respectively the primary and tertiary structure of the antigen binding site. “Multispecificity” as used for the amino acids of the present invention refers to their binding to two or more (structurally) different antigens or antigenic determinants. This multispecificity is achieved by the at least two partially or fully overlapping binding sites or antigen binding sites (“first and second antigen binding sites”) on the amino acids of the invention. The at least two stretches of amino acid residues (that form the two antigen binding sites) on the amino acid sequences of the invention may partially or fully overlap with their primary structure and/or they may partially or fully overlap with their tertiary structure. When the stretches of amino acid residues partially overlap with their primary structure, part of the amino acids that form the primary structure of the first antigen binding site are also the amino acids that form the primary structure of the second antigen binding site. When the stretches of amino acid residues fully overlap with their primary structure, all of the amino acids that form the primary structure of the first antigen binding site are also the amino acids that form the primary structure of the second antigen binding site. When the stretches of amino acid residues partially overlap with their tertiary structure, part of the amino acids that form the tertiary structure of the first antigen binding site are also the amino acids that form the tertiary structure of the second antigen binding site. When the stretches of amino acid residues fully overlap with their tertiary structure, all of the amino acids that form the tertiary structure of the first antigen binding site are also the amino acids that form the tertiary structure of the second antigen binding site.

In the partially overlapping stretches of amino acid residues at least 10%, 20%, 30%, 40%, preferably at least 50%, 60%, 70%, more preferably at least 80%, 85%, 90% or even 95% or more of the amino acid residues that form the primary and/or tertiary structure of the first antigen binding site (i.e. the first stretch of amino acid residues) are also the amino acid residues that form respectively the primary and/or tertiary structure of the second antigen binding site (i.e. the second stretch of amino acid residues). In fully overlapping binding sites, all amino acid residues that form the primary and/or tertiary structure of the first antigen binding site (i.e. the first stretch of amino acid residues) are amino acid residues that form respectively the primary and/or tertiary structure of the second antigen binding site (i.e. the second stretch of amino acid residues).

In a preferred aspect of the invention the binders of the invention have two binding sites that partially or fully overlap with their primary and/or tertiary structure. Such binders are also referred to as “dual specific binders of the invention”. Binders of the invention that have three binding sites that partially or fully overlap with their primary and/or tertiary structure are referred to as “triple specific binders of the invention”. Binders of the invention that have four binding sites that partially or fully overlap with their primary and/or tertiary structure are referred to as “quadruple specific binders of the invention”.

The ability to make a biologically active/functional bispecific or multispecific binder or amino acid sequence consisting of only one binding unit is a remarkable result. The bispecific or multispecific binder(s) or amino acid sequence(s) of the present invention have significant advantages over molecules comprising crosslinked binding units such as the conventional crosslinked Nanobodies, domain antibodies, single domain antibodies, “dAbs”, V_{H} or V_{HH} (i.e. bivalent/bispecific or multivalent/multispecific Nanobodies, domain antibodies, single domain antibodies, “dAbs”, V_{H} or V_{HH}) because they are smaller and more compact in size than the corresponding bivalent/bispecific or multivalent/multispecific Nanobody, domain antibody, single domain antibody, “dAb”, V_{H} or V_{HH} construct. The binders and amino acid sequence(s) of the invention disclosed herein retain the advantageous properties of single Nanobodies, domain antibodies, single domain antibodies, “dAbs”, V_{H} or V_{HH}. By the use of only one binding unit for binding at least two different antigens or antigenic determinants, the overall size of the binder and the chance to form T-cell epitopes is reduced. These binder(s) or amino acid sequence(s) can therefore provide a reduced risk of leading to immunogenicity, thereby reducing the likelihood of immune reactions to the pharmaceutical composition comprising said binder(s) or amino acid sequence(s) of the invention. Furthermore, due to their smaller size, the binder(s) or amino acid sequence(s) of the invention can have enhanced stability when administered (intravenously, orally, etc.) as they will be less susceptible to proteolysis by endogenous proteases than larger, bivalent or multivalent polypeptides.

Also, the amino acid sequences of the invention are easier (and/or cheaper) to produce, express and/or purify than corresponding bispecific constructs comprised of two separate binding units against the respective antigens. It is also envisaged that the amino acid sequences of the invention may be expressed in host cells or host organisms in which the corresponding bispecific construct may not be (efficiently) expressed.

Finally, due to the smaller size of binding molecules with only one binding unit, it may be that pharmaceutical formulations and compositions comprising a binder, amino acid sequence, compound or polypeptide of the invention (and in particular liquid formulations such as solutions) may be more stable under storage and/or easier or cheaper to prepare than equivalent formulations based on the corresponding bivalent or multivalent constructs.

Even in binding molecules or amino acid sequences of the invention with different binding units, (i.e. multivalent binding molecules or amino acid sequences as defined further herein), the use of the bispecific or multispecific binders of the invention or amino acid sequences of the invention might reduce the size of the binding molecules as less binding units are required.

The at least two antigens or antigenic determinants recognized by the multispecific binder of the invention are preferably naturally occurring molecules. Naturally occurring molecules as used in the present invention are molecules that may occur in any suitable species, such as present in a human or animal body or on the body of a human or animal that suffers from a disease or disorder. The molecule may for example be any biological molecule, such as a protein, (poly)peptide, receptor, ligand, antigen, antigenic determinant, enzyme, factor, etc. Examples of these and other suitable naturally occurring binding molecules will be clear to the skilled person based on the disclosure herein.

In a preferred aspect of the invention, said at least two naturally occurring molecules (first and second naturally occurring binding molecules) recognized by the multispecific binder of the invention are themselves capable of binding another (preferably the same) naturally occurring molecule (third naturally occurring binding molecule).

The third naturally occurring binding molecule that is bound by the first and second naturally occurring binding molecules can be a molecule that occurs in any suitable species, such as present in a human or animal body or on the body of a human or animal that suffers from a disease or disorder. The third naturally occurring binding molecule may for example be any biological molecule, such as a protein, (poly)peptide, receptor, ligand, antigen, antigenic determinant, enzyme, factor, etc. Examples of these and other suitable third naturally occurring binding molecules will be clear to the skilled person based on the disclosure herein.

The third naturally occurring binding molecule may independently be an agonist or antagonist for the first naturally occurring binding molecule and the second naturally occurring binding molecule (or the biological action or mechanism in which the first naturally occurring binding molecule and the second naturally occurring binding molecule are involved). Preferably, however, an agonist for both the first and the second naturally occurring binding molecules; or alternatively an antagonist for both the first and the second naturally occurring binding molecules.

In a preferred aspect of the invention, the third naturally occurring binding molecule that interacts with the first and second naturally occurring binding molecules is a binding molecule that (mainly) occurs in circulation and/or that belongs to one of the following classes of biological molecules: cytokines, hormones and chemokines, In another preferred aspect of the invention, the third naturally occurring binding molecule that interacts with the first and second naturally occurring binding molecules is located on the surface of a cell and preferably on at least one or more of the following cells: antigen presenting cells (APC), T-cells, B-cells, Natural killer (NK) cells, macrophages, Dendritic (DC) cells, parenchymal cells, splenocytes, thymocytes, monocytes, lymphoid cells, tumor cells, granulocytes, endothelial cells, epithelial cells, osteoblasts, skin cells, lung cells, colon cells, fibroblasts, Reed-Sternberg cells, peripheral blood lymphocytes, non-lymphoid haematopoietic cells, stromal cells, osteoclasts, hair follicles and brain cells and neurons.

Said at least two naturally occurring binding molecules can be involved in the same or different biological pathways and/or biological mechanisms, but are preferably involved in the same biological pathways and/or biological mechanisms. Said at least two naturally occurring binding molecules may be involved in modulating cellular responses to the third naturally occurring binding molecule. They may be involved in the immune system and/or in modulating the immune response.

In a preferred non-limiting aspect of the invention, at least the first naturally occurring binding molecules and/or at least the second naturally occurring binding molecule (and preferably both the first naturally occurring binding molecule and the second naturally occurring binding molecule) are located on the surface of a cell and preferably on at least one or more of the following cells: antigen presenting cells (APC), T-cells, B-cells, Natural killer (NK) cells, macrophages, Dendritic (DC) cells, parenchymal cells, splenocytes, thymocytes, monocytes, lymphoid cells, tumor cells, granulocytes, endothelial cells, epithelial cells, osteoblasts, skin cells, lung cells, colon cells, fibroblasts, Reed-Sternberg cells, peripheral blood lymphocytes, non-lymphoid haematopoietic cells, stromal cells, osteoclasts, hair follicles and brain cells and neurons.

In a preferred embodiment, the multispecific binder of the invention is directed against a first and a second naturally occurring binding molecule on an antigen presenting cell or on a T-cell.

The multispecific binders of the present invention that bind to at least two naturally occurring binding molecules located on the surface of one or more cells wherein said at least two naturally occurring binding molecules interact with a third naturally occurring binding molecule located on the surface of another cell may modulate (such as blocked or inhibited) the interaction between said one or more cells (containing the at least two naturally occurring binding molecules) and said other cell (containing the third naturally occurring binding molecule). For example (without being limiting), the interaction between following cells may be modulated:
B-cells and T-cells
APCs and T-cells

In another preferred non-limiting aspect of the invention, the first and second naturally occurring binding molecules recognized by the multispecific binder of the invention are receptors or ligands. A receptor is a protein on the cell membrane or within the cytoplasm or cell nucleus that binds to a specific molecule (a ligand), and initiates the cellular response to the ligand. Ligand-induced changes in the behavior of receptor proteins result in physiological changes that constitute the biological actions of the ligands, resulting in the modulation of certain biological pathways and/or certain biological mechanisms, responses and effects associated with such receptor-ligand signalling. When the first and second naturally occurring binding molecules are ligands, the third naturally occurring binding molecule may be their receptor; or when the first and second naturally occurring binding molecules are receptors, the third naturally occurring binding molecule may be their ligand.

The multispecific binders of the present invention that bind at least two receptors or ligands, may modulate these receptor-ligand interaction and can generally be used to modulate, and in particular inhibit and/or prevent, the signalling that is mediated by said receptor-ligand interaction, to modulate the biological pathways in which said receptor-ligand interaction is involved, and/or to modulate the biological mechanisms, responses and effects associated with such signalling or these pathways.

In a preferred aspect of the invention, the first and second naturally occurring binding molecules belong to the same protein superfamily or protein family, such as (without being limiting) the TNF superfamily, the TNFR superfamily, the B7:CD28 superfamily or the Eph family.

In a preferred, but non-limiting, aspect, the at least two naturally occurring binding molecules interact with one of the following third naturally occurring binding molecules:
TNFα
TNFβ
CD95L
VEGI
TRAIL
RANKL
LIGHT
APRIL
BAH
TNFR1
TNFR2
DCR3
OPG
LβR
HVEM
BCMA
TACI
CD28
CTLA4
PD-1
BTLA
CD80
CD86
MHC
EphA1
EphA2
EphA3
EphA4
EphA5
EphA6
EphA7
EphA8
EphB1
EphB2
EphB3
EphB4
EphB5
EphB6
ephrinA1
ephrinA2
ephrinA3
ephrinA4
ephrinA5
ephrinA6
ephrinB1
ephrinB2
ephrinB3

Preferably, without being limiting, the first and second naturally occurring binding molecules are selected from one of the following combinations of first and second naturally binding molecules:
TNFR1, TNFR2 and HVEM;
CD95 and DCR3;
DCR3 and DR3;
DR4, DR5, DCR1, DCR2 and OPG;
OPG and RANKL;
LTβR, DR3 and HVEM;
BCMA, TACI and BAFFR;
TNFα and TNFβ;
CD95L and VEGI;
VEGI and LIGHT;
TRAIL and RANKL;
LTα/LTβ and LIGHT;
TNβ and LIGHT;
APRIL and BAFF
CD80 and CD86
PD-L1 and PD-L2
B7x and B7H-3
CD28 and CTLA-4
TCRαβ and CD4/CD8
Eph A1, Eph A2, Eph A3, Eph A4, Eph A5, Eph A6, Eph A7, Eph A8-Eph B1, Eph B2, Eph B3, Eph B4, Eph B5, Eph B6 ephrinA1, ephrinA2, ephrinA3, ephrinA4, ephrinA5, ephrinA6 ephrinB1, ephrinB2, ephrinB3

Preferably the first and second naturally occurring binding molecules are PD-L1 and PD-L2.

In a preferred, non-limiting, aspect of the invention, the binding to at least two of these naturally occurring binding molecules blocks the interaction of these at least two naturally occurring binding molecules with the third naturally occurring binding molecule. Non-limiting examples of interactions that are blocked by the muitispecific binders of the invention include:
TNFα with TNFR1 and TNFR2;
TNFα with TNFR1, TNFR2 and/or HVEM;
CD95L with CD95 and DCR3;
VEGI with DCR3 and DR3;
TRAIL with DR4, DR5, DCR1, DCR2 and/or OPG;
RANKL with OPG and RANKL;
LIGHT with LTβR, DR3 and/or HVEM;
APRIL with BCMA and TACT;
BAFF with BCMA, TACT and/or BAFFR;
TNFR1 with TNFα and TNFβ;
TNFR2 with TNFα and TNFβ;
DCR3 with CD95L and VEGI;
DR3 with VEGI and LIGHT;
OPG with TRAIL and RANKL;
LTβR with LTα/LTβ and LIGHT;
HVEM with TNFβ and LIGHT;
BCMA with APRIL and BAFF;
TACI with APRIL and BAFF;
CD28 with CD80 and CD86;
CTLA-4 with CD80 and CD86;
PD-1 with PD-L1 and PD-L2;
BTLA with B7x and B7H-3
CD80 with CD28 and CTLA-4;
CD86 with CD28 and CTLA-4;
MHC with TCRαβ and CD4/CD8
EphA1 with ephrinA1, ephrinA2, ephrinA3, ephrinA4, and/or ephrinA5
EphA2 with ephrinA1, ephrinA2, ephrinA3, ephrinA4, ephrinA5, and/or ephrin A6
EphA3 with ephrinA1, ephrinA2, ephrinA3, ephrinA4, ephrinA5, and/or ephrin A6
EphA4 with ephrinA1, ephrinA2, ephrinA3, ephrinA4, ephrinA5, ephrin A6, ephrinB2 and/or ephrinB3
EphA5 with ephrinA1, ephrinA2, ephrinA3, ephrinA4, ephrinA5, and/or ephrin A6
EphA6 with ephrinA1, ephrinA2, ephrinA3, ephrinA4, ephrinA5, and/or ephrin A6
EphA7 with ephrinA1, ephrinA2, ephrinA3, ephrinA4, ephrinA5, and/or ephrin A6
EphA8 with ephrinA1, ephrinA2, ephrinA3, ephrinA4, ephrinA5, and/or ephrin A6
EphB1 with ephrinB1, ephrinB2, and/or ephrinB3
EphB2 with ephrinB1, ephrinB2, and/or ephrinB3
EphB3 with ephrinB1, ephrinB2, and/or ephrinB3
EphB4 with ephrinB1, ephrinB2, and/or ephrinB3
EphB5 with ephrinB1, ephrinB2, and/or ephrinB3
EphB6 with ephrinB1, ephrinB2, and/or ephrinB3
ephrinA1 with EphA1, EphA2, EphA3, EphA4, EphA5, EphA6, EphA7, and/or EphA8
ephrinA2 with EphA1, EphA2, EphA3, EphA4, EphA5, EphA6, EphA7, and/or EphA8
ephrinA3 with EphA1, EphA2, EphA3, EphA4, EphA5, EphA6, EphA7, and/or EphA8
ephrinA4 with EphA1, EphA2, EphA3, EphA4, EphA5, EphA6, EphA7, and/or EphA8
ephrinA5 with EphA1, EphA2, EphA3, EphA4, EphA5, EphA6, EphA7, and/or EphA8
ephrinA6 with EphA2, EphA3, EphA4, EphA5, EphA6, EphA7, and/or EphA8
ephrinB1 with EphB1, EphB2, EphB3, EphB4, EphB5, and/or EphB6
ephrinB2 with EphB1, EphB2, EphB3, EphB4, EphB5, and/or EphB6
ephrinB3 with EphB1, EphB2, EphB3, EphB4, EphB5, and/or EphB6

In a preferred aspect, the dual specific binder of the invention inhibits and/or blocks the interaction of PD-L1 and PD-L2 with PD-1.

The binding molecule as referred to in the present invention can be any molecule known per se that has binding specificity (i.e. multispecific binding by at least two antigen binding sites that partially or fully overlap) of the multispecific binders as described herein. Examples of binding molecules include, without being limiting, molecules based on other protein scaffolds than immunoglobulins including but not limited to protein A domains, tendamistat, fibronectin, lipocalin, CTLA-4, T-cell receptors, designed ankyrin repeats and PDZ domains (Binz et al., Nat. Biotech. 23: 1257, 2005), and binding moieties based on DNA or RNA including but not limited to DNA or RNA aptamers (Ulrich et al., Comb. Chem. High Throughput Screen 9:619-32, 2006).

In a preferred aspect, however, the binder of the invention is an amino acid sequence, preferably an amino acid sequence which comprises an immunoglobulin fold or which, under suitable conditions (such as physiological conditions), is capable of forming an immunoglobulin fold (i.e. by folding), or any fragment thereof. The immunoglobulin protein fold has been used by nature to solve many of the requirements of biomolecular recognition. The immunoglobulin fold occurs in functionally diverse proteins which includes matrix proteins, receptors, chaperones, enzymes and of course antibodies (for a review see Halaby et al., J. Protein Eng. 12: 563-71, 1999). Binding molecules derived from antibodies and antibody fragments are, for example described by Holliger and Hudson, Nature Biotechnology (2005) and include, but are not limited to Fab′ fragments, F(ab′)₂ fragments, Fv fragments, heavy chain variables domains, light chain variable domains, domain antibodies and proteins and peptides suitable for use as domain antibodies, single domain antibodies and proteins and peptides suitable for use as single domain antibodies, Nanobodies® and dAb's; other single variable domains as well as suitable fragments of any of the foregoing, as well as constructs comprising such antibody fragments, binding units or binding molecules (such as ScFv constructs and “diabodies”).

For a general description of (single) domain antibodies, reference is also made to the prior art cited above, as well as to EP 0 368 684. For the term “dAb's”, reference is for example made to Ward et al. (Nature 341: 544-6, 1989), to Holt et al. (Trends Biotechnol. 21: 484-490, 2003; as well as to for example WO 06/030220, WO 06/003388 and other published patent applications of Domantis Ltd. It should also be noted that, although less preferred in the context of the present invention because they are not of mammalian origin, single domain antibodies or single variable domains can be derived from certain species of shark (for example, the so-called “IgNAR domains”, see for example WO 05/18629).

For a general description of heavy chain antibodies and the variable domains thereof, reference is inter alia made to the prior art cited herein, to the review article by Muyldermans in Reviews in Molecular Biotechnology 74 (2001), 277-302; as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. Reference is also made to the further prior art mentioned in these applications, and in particular to the list of references mentioned on pages 41-43 of the International application WO 06/040153, which list and references are incorporated herein by reference.

In accordance with the terminology used in the art (see the above references), the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and Nanobodies can function as a single, relatively small, functional antigen-binding structural unit, domain or protein. This distinguishes the V_{HH} domains from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a V_{H} domain covalently linked to a V_{L} domain).

Because of these unique properties, the use of V_{HH} domains and Nanobodies as single antigen-binding proteins or as antigen-binding domains (i.e. as part of a larger protein or polypeptide) offers a number of significant advantages over the use of conventional V_{H} and V_{L} domains, scFv's or conventional antibody fragments (such as Fab- or F(ab′)₂-fragments):
only a single domain is required to bind an antigen with high affinity and with high selectivity, so that there is no need to have two separate domains present, nor to assure that these two domains are present in the right spacial conformation and configuration (i.e. through the use of especially designed linkers, as with scFv's);
V_{HH} domains and Nanobodies can be expressed from a single gene and require no post-translational folding or modifications;
V_{HH} domains and Nanobodies can easily be engineered into multivalent multispecific formats (as further discussed herein);
V_{HH} domains and Nanobodies are highly soluble and do not have a tendency to aggregate (as with the mouse-derived “dAb's” described by Ward et al., Nature, Vol. 341, 1989, p. 544);
V_{HH} domains and Nanobodies are highly stable to heat, pH, proteases and other denaturing agents or conditions (see for example Ewert et al, supra);
V_{HH} domains and Nanobodies are easy and relatively cheap to prepare, even on a scale required for production. For example, V_{HH} domains, Nanobodies and proteins/polypeptides containing the same can be produced using microbial fermentation (e.g. as further described below) and do not require the use of mammalian expression systems, as with for example conventional antibody fragments;
V_{HH} domains and Nanobodies are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) than such conventional 4-chain antibodies and antigen-binding fragments thereof;
V_{HH} domains and Nanobodies can show so-called cavity-binding properties (inter alia due to their extended CDR3 loop, compared to conventional V_{H} domains) and can therefore also access targets and epitopes not accessable to conventional 4-chain antibodies and antigen-binding fragments thereof. For example, it has been shown that V_{HH} domains and Nanobodies can inhibit enzymes (see for example WO 97/49805; Transue et al., Proteins 32: 515-22, 1998; Lauwereys et al., EMBO J. 17: 3512-20, 1998).

In a specific and preferred aspect, the invention provides multispecific Nanobodies against at least two antigens or antigenic determinants, such as the at least two naturally occurring binding molecules; and in particular Nanobodies against at least two binding molecules naturally occurring in a warm-blooded animal, and more in particular Nanobodies against at least two binding molecules naturally occurring in a mammal, and especially Nanobodies against at least two binding molecules naturally occurring in humans; as well as proteins and/or polypeptides comprising at least one such Nanobody. The multispecific Nanobodies of the invention comprise at least two antigen binding sites that are partially or fully overlapping in their primary and/or tertiary structure as defined herein.

For a general description of Nanobodies as well as some of the further terms used in the present description including “humanization” and/or “camelization” of Nanobodies, as well as other modifications, parts or fragments, derivatives or “Nanobody fusions”, reference is made to the further description below, as well as to the prior art cited herein. As mentioned in said prior art, Nanobodies can in particular be characterized by the presence of one or more “Hallmark residues” (see WO 06/040153, WO 06/079372, WO 06/122786, WO 06/122787, WO 06/122825, WO 07/104,529, WO 07/118,670 and WO 07/042,289) in one or more of the framework sequences. A detailed description of the preparation of Nanbodies can be found in WO 06/040153 as well.

The total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein.

The amino acid residues of a Nanobody are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication); or referred to herein. According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.].

Alternative methods for numbering the amino acid residues of V₁₁ domains, which methods can also be applied in an analogous manner to V_{HH} domains from Camelids and to Nanobodies, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed. unless indicated otherwise.

Generally, it should be noted that the term Nanobody as used herein in its broadest sense is not limited to a specific biological source or to a specific method of preparation. For example, as will be discussed in more detail below, the Nanobodies of the invention can generally be obtained: (1) by isolating the V_{HH} domain of a naturally occurring heavy chain antibody; (2) by expression of a nucleotide sequence encoding a naturally occurring V_{HH} domain; (3) by “humanization” (as described herein) of a naturally occurring V_{HH} domain or by expression of a nucleic acid encoding a such humanized V_{HH} domain; (4) by “camelization” (as described herein) of a naturally occurring V_{H} domain from any animal species, and in particular a from species of mammal, such as from a human being, or by expression of a nucleic acid encoding such a camelized V_{H} domain; (5) by “camelisation” of a “domain antibody” or “Dab” as described by Ward et al (supra), or by expression of a nucleic acid encoding such a camelized V_{H} domain; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. Suitable methods and techniques for performing the foregoing will be clear to the skilled person based on the disclosure herein and for example include the methods and techniques described in more detail herein.

In a Nanobody of the invention, the binding sites for binding to the at least two naturally occurring binding molecules are preferably formed by the CDR sequences. Optionally, the Nanobody of the invention may also, and in addition to the at least two binding sites for binding to the at least two naturally occurring binding molecules, contain one or more further binding sites for binding against other antigens, proteins or targets. For methods and positions for introducing such second binding sites, reference is for example made to Keck and Huston, Biophysical Journal, 71, October 1996, 2002-2011; EP 0 640 130; WO 06/07260.

As generally described herein for the amino acid sequences of the invention, the Nanobodies of the invention are preferably in essentially isolated form (as defined herein), or form part of a protein or polypeptide of the invention (as defined herein), which may comprise or essentially consist of one or more Nanobodies of the invention and which may optionally further comprise one or more further amino acid sequences (all optionally linked via one or more suitable linkers). For example, and without limitation, the Nanobody of the invention may be used as a binding unit in such a protein or polypeptide, which may optionally contain one or more further amino acid sequences that can serve as a binding unit (i.e. against one or more other targets than the first and second naturally occurring binding molecules) so as to provide a monovalent multispecific or multivalent multispecific polypeptide of the invention, respectively, all as described herein. In particular, such a protein or polypeptide may comprise or essentially consist of one or more Nanobodies of the invention and optionally one or more (other) Nanobodies (i.e. directed against other targets than the first and second naturally occurring binding molecules), all optionally linked via one or more suitable linkers, so as to provide a monovalent multispecific or multivalent multispecific Nanobody construct, respectively, as further described herein. Such proteins or polypeptides may also be in essentially isolated form (as defined herein).

The invention also provides polypeptides or compounds (also called polypeptides or compounds of the invention) that comprise or essentially consist of a Nanobody, an amino acid sequence or a binder of the invention as disclosed herein. By “essentially consist of” is meant that the compound or the polypeptide of the invention either is exactly the same as the binder, the amino acid sequence or the Nanobody of the invention or corresponds to the binder, the amino acid sequence or the Nanobody of the invention. The amino acid sequence or Nanobody of the invention may have a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence or the Nanobody.

Said amino acid residues may or may not change, alter or otherwise influence the (biological) properties of the amino acid sequence or Nanobody and may or may not add further functionality to the amino acid sequence or Nanobody. For example, such amino acid residues:
can comprise an N-terminal Met residue, for example as result of expression in a heterologous host cell or host organism.
may form a signal sequence or leader sequence that directs secretion of the amino acid sequence or Nanobody from a host cell upon synthesis. Suitable secretory leader peptides will be clear to the skilled person, and may be as further described herein. Usually, such a leader sequence will be linked to the N-terminus of the amino acid sequence or Nanobody, although the invention in its broadest sense is not limited thereto;
may form a sequence or signal that allows the amino acid sequence or Nanobody to be directed towards and/or to penetrate or enter into specific organs, tissues, cells, or parts or compartments of cells, and/or that allows the amino acid sequence or Nanobody to penetrate or cross a biological barrier such as a cell membrane, a cell layer such as a layer of epithelial cells, a tumor including solid tumors, or the blood-brain-barrier. Examples of such amino acid sequences will be clear to the skilled person. Some non-limiting examples are the small peptide vectors (“Pep-trans vectors”) described in WO 03/026700 and in Temsamani et al., Expert Opin. Biol. Ther., 1, 773 (2001); Temsamani and Vidal, Drug Discov. Today, 9, 1012 (004) and Rousselle, J. Pharmacol. Exp. Ther., 296, 124-131 (2001), and the membrane translocator sequence described by Zhao et al., Apoptosis, 8, 631-637 (2003). C-terminal and N-terminal amino acid sequences for intracellular targeting of antibody fragments are for example described by Cardinale et al., Methods, 34, 171 (2004). Other suitable techniques for intracellular targeting involve the expression and/or use of so-called “intrabodies” comprising an amino acid sequence or Nanobody of the invention, as mentioned below; may form a “tag”, for example an amino acid sequence or residue that allows or facilitates the purification of the amino acid sequence or Nanobody, for example using affinity techniques directed against said sequence or residue. Thereafter, said sequence or residue may be removed (e.g. by chemical or enzymatical cleavage) to provide the amino acid sequence or Nanobody sequence (for this purpose, the tag may optionally be linked to the amino acid sequence or Nanobody sequence via a cleavable linker sequence or contain a cleavable motif). Some preferred, but non-limiting examples of such residues are multiple histidine residues, glutatione residues and a myc-tag (see for example SEQ ID NO:31 of WO 06/12282).
may be one or more amino acid residues that have been functionalized and/or that can serve as a site for attachment of functional groups. Suitable amino acid residues and functional groups will be clear to the skilled person and include, but are not limited to, the amino acid residues and functional groups mentioned herein for the derivatives of the amino acid sequences or Nanobodies of the invention.

According to another aspect, a compound or polypeptide of the invention comprises a binder, amino acid sequence or Nanobody of the invention, which is fused (at its amino terminal end, at its carboxy terminal end, or both at its amino terminal end and at its carboxy terminal end) to at least one further amino acid sequence, i.e. so as to provide a fusion protein comprising said binder, amino acid sequence or Nanobody of the invention and the one or more further amino acid sequences.

The one or more further amino acid sequence may be any suitable and/or desired amino acid sequences. The further amino acid sequences may or may not change, alter or otherwise influence the (biological) properties of the binder, amino acid sequence or Nanobody of the invention, and may or may not add further functionality to the binder, amino acid sequence or Nanobody or the polypeptide of the invention. Preferably, the further amino acid sequence is such that it confers one or more desired properties or functionalities to the binder, amino acid sequence, Nanobody, compound or the polypeptide of the invention, such as, for example, increases the half-life, the solubility, or the absorption, reduces the immunogenicity or the toxicity, eliminates or attenuates undesirable side effects, and/or confers other advantageous properties to and/or reduces the undesired properties of the compounds or polypeptides of the invention, compared to the binders, amino acid sequences or Nanobody of the invention per se. Some non-limiting examples of such fusion constructs will become clear from the further description herein.

In another aspect of the invention, the compound or polypeptide of the invention comprises a binder, amino acid sequence or Nanobody of the invention, which is fused to a further binding unit that provides an additional binding site, which binding site may be directed against any desired protein, polypeptide, antigen, antigenic determinant or epitope (including but not limited to one or more of the same proteins, polypeptides, antigens, antigenic determinants or epitopes against which the binders, amino acid sequences or Nanobody of the invention are directed, or a different protein, polypeptide, antigen, antigenic determinant or epitope).

By “binding unit” is meant in this description any amino acid sequence, peptide, protein, polypeptide, construct, fusion protein, compound, factor or other entity capable of binding an antigen or antigenic determinant as described herein, such as a binder or an amino acid sequence of the invention. When a compound, protein, polypeptide or construct comprises two or more binding units, said binding units may optionally be linked to each other via one or more suitable linkers. Example of binding units will be clear to the skilled person, and may generally comprise all amino acid sequences that are used in peptide fusions based on conventional antibodies and fragments thereof (including but not limited to ScFv's and single domain antibodies). Reference is for example made to the review by Holliger and Hudson, Nature Biotechnology, 23, 9, 1126-1136 (2005).

According to another aspect, the one or more further binding unit may comprise one or more parts, fragments or domains of conventional 4-chain antibodies (and in particular human antibodies) and/or of heavy chain antibodies. For example, although usually less preferred, an amino acid sequence or Nanobody of the invention may be linked to a conventional (preferably human) V_{H} or V_{L} domain or to a natural or synthetic analog of a V_{H} or V_{L} domain (including but not limited to other (single) domain antibodies. such as the dAb's described by Ward et al.), again optionally via a linker sequence.

Generally, polypeptides or compound that comprise or essentially consist of a single binding unit such as an amino acid sequence or binder of the invention will be referred to herein as “monovalent” polypeptides or compounds or as “monovalent constructs”. Polypeptides or proteins that comprise or essentially consist of two or more binding unit such as two amino acid sequence(s) and/or binders of the invention, or one amino acid sequence or binder of the invention and one or more other antigen binding domains such as a Nanobody will be referred to herein as “multivalent” polypeptides or proteins or as “multivalent constructs”, and these may, in specific situations, provide certain advantages compared to the corresponding monovalent polypeptide or compounds of the invention.

All polypeptides or compounds encompassed within the scope of the present invention are multispecific (as defined herein) and contain at least one binding unit that comprises at least two partially or fully overlapping antigen binding sites. These polypeptides or compounds may comprise one or more additional binding units that confers additional antigen specificity to said compound or polypeptide. Therefore, according to another specific, but non-limiting embodiment, a polypeptide or compound of the invention may comprise or essentially consists of at least one amino acid sequence or binder of the invention, optionally one or more further amino acid sequence or binder of the invention, and at least one other binding unit (such as an amino acid sequence) that confers additional specificity to the polypeptide or compound of the invention.

It is also possible to combine two or more of the above embodiments, for example to provide a trivalent polypeptide or compound comprising two amino acid sequence(s) and/or binders of the invention and one or more other binding unit or antigen binding domains such as a Nanobody; or to provide a trivalent polypeptide comprising one amino acid sequence or binder of the invention and two other binding units or antigen binding domains such as a Nanobodies. Further non-limiting examples of such polypeptides and compounds, as well as some polypeptides and compounds that are particularly preferred within the context of the present invention, will become clear from the further description herein.

In one specific aspect of the invention, a compound or polypeptide of the invention comprising at least one binder, amino acid sequence or Nanobody of the invention may have an increased half-life, compared to the corresponding binder, amino acid sequence or Nanobody of the invention. For example, the binders, amino acid sequences or Nanobodies of the invention can be chemically modified to increase the half-life thereof (for example, by means of pegylation); they can be linked to serum proteins, such as human serum albumin (see for example WO 00/27435) or haptenic molecules (for example haptens that are recognized by circulating antibodies, see for example WO 98/22141).

In particular, it has been described in the art that linking fragments of immunoglobulins (such as V_{H} domains) to serum albumin or to fragments thereof can be used to increase the half-life. Reference is for made to WO 00/27435 and WO 01/077137. According to the invention, the binder, amino acid sequence or Nanobody of the invention is preferably either directly linked to serum albumin (or to a suitable fragment thereof) or via a suitable linker, and in particular via a suitable peptide linked so that the compound or polypeptide of the invention can be expressed as a genetic fusion (protein). According to one specific aspect, the binder, amino acid sequence or Nanobody of the invention may be linked to a fragment of serum albumin that at least comprises the domain III of serum albumin or part thereof. Reference is for example made to the U.S. provisional application 60/788,256 of Ablynx N.V. entitled “Albumin derived amino acid sequence, use thereof for increasing the half-life of therapeutic proteins and of other therapeutic proteins and entities, and constructs comprising the same” filed on Mar. 31, 2006 (see also PCT/EP2007/002817).

The binder, amino acid sequence or Nanobody of the invention may also be linked to one or more (preferably human) C_{H}1, C_{H}2 and/or C_{H}3 domains, optionally via a linker sequence. For instance, a Nanobody linked to a suitable C_{H}1 domain could for example be used—together with suitable light chains—to generate antibody fragments/structures analogous to conventional Fab fragments or F(ab′)₂ fragments, but in which one or (in case of a F(ab′)₂ fragment) one or both of the conventional. V_{H} domains have been replaced by a Nanobody of the invention. Also, two binders, amino acid sequences or Nanobodies could be linked to a C_{H}3 domain (optionally via a linker) to provide a construct with increased half-life in vivo.

The binder, amino acid sequence or Nanobody of the invention could be linked to at least one additional binding site that is directed against a serum protein (such as, for example, human serum albumin or another serum protein such as IgG), so as to provide increased half-life in serum. Such amino acid sequences for example include the Nanobodies described below, as well as the small peptides and binding proteins described in WO 91/01743, WO 01/45746 and WO 02/076489 and the dAb's described in WO 03/002609 and WO 04/003019. Reference is also made to Harmsen et al., Vaccine, 23 (41); 4926-42, 2005, as well as to EP 0 368 684, as well as to the following the U.S. provisional applications 60/843,349 (see also PCT/EP2007/059475), 60/850,774 (see also PCT/EP2007/060849), 60/850,775 775 (see also PCT/EP2007/060850) by Ablynx N.V. mentioned herein and US provisional application of Ablynx N.V. entitled “Peptides capable of binding to serum proteins” filed on Dec. 5, 2006 (see also PCT/EP/2007/063348).

Such amino acid sequences may in particular be directed against serum albumin (and more in particular human serum albumin) and/or against IgG (and more in particular human IgG). For example, such amino acid sequences may be amino acid sequences that are directed against (human) serum albumin and amino acid sequences that can bind to amino acid residues on (human) serum albumin that are not involved in binding of serum albumin to FcRn (see for example WO 06/0122787) and/or amino acid sequences that are capable of binding to amino acid residues on serum albumin that do not form part of domain III of serum albumin (see again for example WO 06/0122787); amino acid sequences that have or can provide an increased half-life (see for example the U.S. provisional application 60/843,349 by Ablynx N.V. entitled “Serum albumin binding proteins with long half-lives” filed on Sep. 8, 2006; see also PCT/EP2007/059475); amino acid sequences against human serum albumin that are cross-reactive with serum albumin from at least one species of mammal, and in particular with at least one species of primate (such as, without limitation, monkeys from the genus Macaca (such as, and in particular, cynomologus monkeys (Macaca fascicularis) and/or rhesus monkeys (Macaca mulatta)) and baboon (Papio ursinus), reference is again made to the U.S. provisional application 60/843,349 and PCT/EP2007/059475); amino acid sequences that can bind to serum albumin in a pH independent manner (see for example the U.S. provisional application 60/850,774 by Ablynx N.V. entitled “Amino acid sequences that bind to serum proteins in a manner that is essentially independent of the pH, compounds comprising the same, and uses thereof”, filed on Oct. 11, 2006; see also and PCT/EP2007/059475) and/or amino acid sequences that are conditional binders (see for example the U.S. provisional application 60/850,775 by Ablynx N.V. entitled “Amino acid sequences that bind to a desired molecule in a conditional manner”, filed on Oct. 11, 2006; see also PCT/EP2007/060850).

Generally, the binders, amino acid sequences or Nanobodies of the invention (or compounds or polypeptides comprising the same) with increased half-life preferably have a half-life that is at least 1.5 times, preferably at least 2 times, such as at least 5 times, for example at least 10 times or more than 20 times, greater than the half-life of the corresponding binder, amino acid sequence or Nanobody of the invention per se. For example, the binders, amino acid sequences, Nanobodies, compounds or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding binder, amino acid sequence or Nanobody of the invention per se.

In a preferred, but non-limiting aspect of the invention, such binders, amino acid sequences or Nanobodies of the invention have a serum half-life that is increased with more than 1 hours, preferably more than 2 hours, more preferably more than 6 hours, such as more than 12 hours, or even more than 24, 48 or 72 hours, compared to the corresponding binder or amino acid sequence of the invention per se.

In a preferred, but non-limiting aspect of the invention, such Nanobodies, compound, constructs or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another aspect of the invention, a compound or polypeptide of the invention comprises one or more (such as two or preferably one) binders, amino acid sequences and/or Nanobodies of the invention linked (optionally via one or more suitable linker sequences) to one or more (such as two and preferably one) amino acid sequences that allow the resulting polypeptide of the invention to cross the blood brain barrier. In particular, said one or more amino acid sequences that allow the resulting polypeptides of the invention to cross the blood brain barrier may be one or more (such as two and preferably one) Nanobodies, such as the Nanobodies described in WO 02/057445, of which FC44 (SEQ ID NO: 189 of WO 06/040153) and FC5 (SEQ ID NO: 190 of WO 06/040154) are preferred examples.

According to yet another aspect of the invention, one or more binders, amino acid sequences or Nanobodies of the invention may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the compound or polypeptide of the invention and/or may confer the ability to bind to one or more Fc receptors. For example, for this purpose, and without being limited thereto, the one or more further amino acid sequences may comprise one or more C_{H}2 and/or C_{H}3 domains of an antibody, such as from a heavy chain antibody (as described herein) and more preferably from a conventional human 4-chain antibody; and/or may form (part of) an Fc region, for example from IgG (e.g. from IgG1, IgG2, IgG3 or IgG4), from IgE or from another human Ig such as IgA, IgD or IgM. For example, WO 94/04678 describes heavy chain antibodies comprising a Camelid V_{HH} domain or a humanized derivative thereof (i.e. a Nanobody), in which the Camelidae C_{H}2 and/or C_{H}3 domain have been replaced by human C_{H}2 and C_{H}3 domains, so as to provide an immunoglobulin that consists of 2 heavy chains each comprising a Nanobody and human C_{H}2 and C_{H}3 domains (but no C_{B}1 domain), which immunoglobulin has the effector function provided by the C_{H}2 and C_{H}3 domains and which immunoglobulin can function without the presence of any light chains. Other amino acid sequences that can be suitably linked to the binders, amino acid sequences or Nanobodies of the invention so as to provide an effector function will be clear to the skilled person, and may be chosen on the basis of the desired effector function(s). Reference is for example made to WO 04/058820, WO 99/42077, WO 02/056910 and WO 05/017148, as well as the review by Holliger and Hudson, supra; and to the non-prepublished US provisional application by Ablynx N.V. entitled “Constructs comprising single variable domains and an Fc portion derived from IgE” which has a filing date of Dec. 4, 2007. Coupling of a binder, amino acid sequence or Nanobody of the invention to an Fc portion may also lead to an increased half-life, compared to the corresponding binder, amino acid sequence or Nanobody of the invention. For some applications, the use of an Fc portion and/or of constant domains (i.e. C_{H}2 and/or C_{H}3 domains) that confer increased half-life without any biologically significant effector function may also be suitable or even preferred. Other suitable constructs comprising one or more binders, amino acid sequences or Nanobodies and one or more constant domains with increased half-life in vivo will be clear to the skilled person, and may for example comprise two Nanobodies linked to a C_{H}3 domain, optionally via a linker sequence. Generally, any fusion protein or derivatives with increased half-life will preferably have a molecular weight of more than 50 kD, the cut-off value for renal absorption.

In another one specific, but non-limiting, aspect, in order to form a polypeptide of the invention, one or more one or more binders, amino acid sequences or Nanobodies of the invention may be linked (optionally via a suitable linker or hinge region) to naturally occurring, synthetic or semisynthetic constant domains (or analogs, variants, mutants, parts or fragments thereof) that have a reduced (or essentially no) tendency to self-associate into dimers (i.e. compared to constant domains that naturally occur in conventional 4-chain antibodies). Such monomeric (i.e. not self-associating) Fc chain variants, or fragments thereof, will be clear to the skilled person. For example, Helm et al., J Biol Chem 1996 271 7494, describe monomeric Fcc chain variants that can be used in the polypeptide chains of the invention.

Also, such monomeric Fc chain variants are preferably such that they are still capable of binding to the complement or the relevant Fc receptor(s) (depending on the Fc portion from which they are derived), and/or such that they still have some or all of the effector functions of the Fc portion from which they are derived (or at a reduced level still suitable for the intended use). Alternatively, in such a polypeptide chain of the invention, the monomeric Fc chain may be used to confer increased half-life upon the polypeptide chain, in which case the monomeric Fc chain may also have no or essentially no effector functions.

The further amino acid sequences may also form a signal sequence or leader sequence that directs secretion of the amino acid sequence, Nanobody or polypeptide of the invention from a host cell upon synthesis (for example to provide a pre-, pro- or prepro-form of the polypeptide of the invention, depending on the host cell used to express the polypeptide of the invention).

The further amino acid sequence may also form a sequence or signal that allows the binder, amino acid sequence, Nanobody, compound or polypeptide of the invention to be directed towards and/or to penetrate or enter into specific organs, tissues, cells, or parts or compartments of cells, and/or that allows the binder, amino acid sequence, Nanobody, compound or polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, the “Peptrans” vectors mentioned above, the sequences described by Cardinale et al. and the amino acid sequences and antibody fragments known per se that can be used to express or produce the amino acid sequences, Nanobodies and polypeptides of the invention as so-called “intrabodies”, for example as described in WO 94/02610, WO 95/22618, U.S. Pat. No. 7,004,940, WO 03/014960, WO 99/07414; WO 05/01690; EP 1 512 696; and in Cattaneo, A. & Biocca, S. (1997) Intracellular Antibodies: Development and Applications. Landes and Springer-Verlag; and in Kontermann, Methods 34, (2004), 163-170, and the further references described therein.

For some applications, in particular for those applications in which it is intended to kill a cell that expresses the target against which the binder, amino acid sequence or Nanobodies of the invention are directed (e.g. in the treatment of cancer), or to reduce or slow the growth and/or proliferation of such a cell, the binder, amino acid sequence or Nanobodies of the invention may also be linked to a (cyto)toxic protein or polypeptide. Examples of such toxic proteins and polypeptides which can be linked to a binder, amino acid sequence or Nanobody of the invention to provide—for example—a cytotoxic compound, polypeptide of the invention will be clear to the skilled person and can for example be found in the prior art cited above and/or in the further description herein. One example is the so-called ADEPT™ technology described in WO 03/055527.

In the above polypeptides and compounds, the one or more amino acid sequence or binders of the invention and/or other amino acid sequence may be directly linked or linked via one or more linker sequences.

Suitable spacers or linkers for use in multivalent compounds or polypeptides of the invention will be clear to the skilled person, and may generally be any linker or spacer used in the art to link binding molecules. Preferably, said linker or spacer is suitable for use in constructing compounds or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, its should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the invention, since each amino acid sequence or Nanobody by itself forms at least two complete antigen-binding sites).

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser_{y}), such as (for example (gly₄ser)₃ or (gly₃ser₂)₃, as described in. WO 99/42077 and the GS30, GS15, GS9 and GS7 linkers described in the applications by Ablynx mentioned herein (see for example WO 06/040153 and WO 06/122825), as well as hinge-like regions, such as the hinge regions of naturally occurring heavy chain antibodies or similar sequences (such as described in. WO 94/04678).

Some other particularly preferred linkers are poly-alanine (such as AAA), as well as the linkers GS35, GS30 (SEQ ID NO: 85 in WO 06/122825) and GS9 (SEQ ID NO: 84 in WO 06/122825).

Other suitable linkers generally comprise organic compounds or polymers, in particular those suitable for use in proteins for pharmaceutical use. For instance, polyethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final compound or polypeptide of the invention, including but not limited to the affinity, specificity or avidity for the at least two naturally occurring binding molecules, or for one or more of the other antigens. Based on the disclosure herein, the skilled person will be able to determine the optimal linker(s) for use in a specific compound or polypeptide of the invention, optionally after some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the binders, amino acids and Nanobodies of the invention). For example, linkers containing one or more charged amino acid residues (see Table A-2 above) can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific compound or polypeptide of the invention, optionally after some limited routine experiments.

Finally, when two or more linkers are used in the polypeptides of the invention, these linkers may be the same or different. Again, based on the disclosure herein, the skilled person will be able to determine the optimal linkers for use in a specific compound or polypeptide of the invention, optionally after some limited routine experiments.

In another aspect, the invention relates to host or host cell that expresses or that is capable of expressing a binders, amino acid sequence and/or Nanobody (or nucleic acid encoding the same) of the invention and/or a polypeptide of the invention comprising the same; and/or that contains a nucleic acid of the invention. Some preferred but non-limiting examples of such hosts or host cells will become clear from the further description herein.

Another aspect of the invention relates to a product or composition containing or comprising at least one binder, amino acid sequence and/or Nanobodies of the invention and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention further relates to methods for preparing or generating the binders, amino acid sequences, Nanobodies (or nucleic acids encoding the same), compounds, constructs, polypeptides, nucleic acids, host cells, products and compositions described herein. Some preferred but non-limiting examples of such methods will become clear from the further description herein.

In a specific aspect of the invention, the binders, amino acid sequences and/or Nanobodies of the invention (or nucleic acids encoding the same) are obtainable by at least two rounds of selection or screening on the at least two different antigens, such as a first round of selection or screening on the first naturally occurring binding molecule and a second round of selection or screening on the second naturally occurring binding molecule.

The method for generating the multispecific binders, amino acid sequences and/or Nanobodies of the invention therefore may comprise the steps of:
a) providing a set, collection or library of amino acid sequences and/or Nanobodies; and
b) screening said set, collection or library of amino acid sequences and/or Nanobodies for amino acid sequences and/or Nanobodies that can bind to and/or have affinity for the first naturally occurring binding molecule;
c) screening said set, collection or library of amino acid sequences and/or Nanobodies for amino acid sequences and/or Nanobodies that can bind to and/or have affinity for the second naturally occurring binding molecule;
   and
d) isolating the amino acid sequence(s) and/or Nanobody(ies) that can bind to and/or have affinity for said first and said second naturally occurring binding molecule.

In particular, such a method can comprise the steps of:
a) providing a set, collection or library of amino acid sequences and/or Nanobodies; and
b) screening said set, collection or library of amino acid sequences and/or Nanobodies for amino acid sequences and/or Nanobodies that can bind to and/or have affinity for the first naturally occurring binding molecule;
c) screening the amino acid sequences and/or Nanobodies obtained in step b) for amino acid sequences and/or Nanobodies that can bind to and/or have affinity for the second naturally occurring binding molecule;
   and
d) isolating the amino acid sequence(s) and/or Nanobody(ies) that can bind to and/or have affinity for said first and said second naturally occurring binding molecule.

The term “screening” as used in the present description can comprise selection, screening or any suitable combination of selection and/or screening techniques. Screening of amino acid sequences that can bind to and/or have affinity to the selected antigen can be done by techniques known per se for measuring antigen binding. Conventional antigen binding assays include but are not limited to Scatchard analysis, fluid or gel precipitation reactions, immunodiffusion (single or double), agglutination assays, immunoelectrophoresis, radioimmunoassays (RIA), enzyme immunoassays (EIA) and sandwich competition assays, enzyme-linked immunosorbent assays (ELISA), Western blots, liposome immunoassays (Monroe et al., 1986), complement-fixation assays, immunoradiometric assays, fluorescent immunoassays, protein A immunoassays, or immunoPCR and the different variants thereof known per se in the art, as well as fluorescence based techniques, including FRET, or techniques such as surface plasmon resonance which measure the mass of molecules. An overview of different assays is given in Wild D. (ed. 2001, The Immunoassay Handbook 2^{nd} edition. Nature Pr., London, UK) and Ghindilis et al. (eds. 2002, Immunoassay Methods and Protocols. Humana Press, Totowa, N.J., US). The bispecificity and multispecificity of the amino acid sequence of the invention can also be readily tested using sandwich binding tests based on technologies as surface plasmon resonance and ELISA.

If desired, an additional isolation step can be included between the two screening steps, isolating the amino acid sequence(s) that can bind to and/or have affinity for said first naturally occurring binding molecule.

For example, the screening process can easily be performed comprising the steps of:
i) providing a suitable carrier or support (such as a column, beads, or solid surface such as the surface of a well of a multi-well plate, or the stationary phase of a Biacore) onto which the first naturally occurring binding molecule is suitably immobilized (for example covalently or via an avidin-steptavidin linkage);
ii) contacting said carrier or support with the set, collection or library of amino acid sequences;
iii) washing away the amino acid sequences that do not bind to the first naturally occurring binding molecule bound to said carrier or support;
iv) eluting the amino acid sequences that did bind to the first naturally occurring binding molecule;
v) providing a second suitable carrier or support (such as a column, beads, or solid surface such as the surface of a well of a multi-well plate, or the stationary phase of a Biacore) onto which the second naturally occurring binding molecule is suitably immobilized (for example covalently or via an avidin-steptavidin linkage);
vi) contacting said second carrier or support with the amino acid sequences elated in step iv);
vii) washing away the amino acid sequences that do not bind to the second naturally occurring binding molecule bound to said carrier or support; and
viii) collecting the amino acid sequences that bind to the first and second naturally occurring binding molecule.

In a preferred aspect of the invention, the set, collection or library of amino acid sequences is screened for amino acid sequences that can bind to the first naturally occurring binding molecule with a dissociation constant (K_{D}), a kₒₙ-rate and/or a k_{off}-rate that approximates the dissociation constant (K_{D}), kₒₙ-rate and/or k_{off}-rate with which said amino acid sequence can bind to the second naturally occurring binding molecule. In another preferred aspect of the invention, the set, collection or library of amino acid sequences is screened for amino acid sequences that can bind to the first naturally occurring binding molecule with a dissociation constant (K_{D}), a kₒₙ-rate and/or a k_{off}-rate that is at least 2 fold more, at least 5 fold more, at least 10 fold more, preferably at least 100 fold more, more preferably at least 1000 fold more, than the dissociation constant (K_{D}), kₒₙ-rate and/or k_{off}-rate with which said amino acid sequence can bind to the second naturally occurring binding molecule. In another preferred aspect of the invention, the set, collection or library of amino acid sequences is screened for amino acid sequences that can bind to the second naturally occurring binding molecule with a dissociation constant (K_{D}), a kₒₙ-rate and/or a k_{off}-rate that is at least 2 fold more, at least 5 fold more, at least 10 fold more, preferably at least 100 fold more, more preferably at least 1000 fold more, than the dissociation constant (K_{D}), kₒₙ-rate and/or k_{off}-rate with which said amino acid sequence can bind to the first naturally occurring binding molecule.

In another preferred aspect of the invention, the set, collection or library of amino acid sequences is screened for amino acid sequences that can bind to the first naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said first naturally occurring binding molecule. In another preferred aspect of the invention, the set, collection or library of amino acid sequences is screened for amino acid sequences that can bind to the second naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said second naturally occurring binding molecule. In another preferred aspect of the invention, the set, collection or library of amino acid sequences is screened for amino acid sequences that can bind to the first naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said first naturally occurring binding molecule and that can bind to the second naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said second naturally occurring binding molecule.

In such a method, the set, collection or library of amino acid sequences may be any suitable set, collection or library of amino acid sequences. For example, the set, collection or library of amino acid sequences may be a set, collection or library of immunoglobulin sequences (as described herein), such as a naïve set, collection or library of immunoglobulin sequences; a synthetic or semi-synthetic set, collection or library of immunoglobulin sequences; and/or a set, collection or library of immunoglobulin sequences that have been subjected to affinity maturation.

Also, in such a method, the set, collection or library of amino acid sequences may be a set, collection or library of heavy chain variable domains (such as V_{H} domains or V_{HH} domains) or of light chain variable domains. For example, the set, collection or library of amino acid sequences may be a set, collection or library of domain antibodies or single domain antibodies, or may be a set, collection or library of amino acid sequences that are capable of functioning as a domain antibody or single domain antibody.

In a preferred aspect of this method, the set, collection or library of amino acid sequences may be an immune set, collection or library of immunoglobulin sequences, for example derived from a mammal that has been suitably immunized with the first and/or second naturally occurring binding molecule or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example on the basis of the further disclosure herein. Reference is also made to the review by Hoogenboom in Nature Biotechnology, 23, 9, 1105-1116 (2005).

The set, collection or library may contain any suitable number of amino acid sequences, such as 1, 2, 3 or about 5, 10, 50, 100, 500, 1000, 5000, 10⁴, 10⁵, 10⁶, 10⁷, 10⁸ or more sequences.

The above set, collection or library of amino acid sequences may contain one or more sequences that are not known in advance of the selection and or screening process for example if these sequences are the result of a randomization step (e.g. via error-prone PCR or other means) of one or more given amino acid sequences. Also, one or more or all of the amino acid sequences in the above set, collection or library of amino acid sequences may be obtained or defined by rational, or semi-empirical approaches such as computer modelling techniques or biostatics or data-mining techniques wherein amino acid sequences may have been defined or proposed that are predicted or expected to be endowed with certain properties such as increased stability, pH optimum, protease sensitivity or other properties or combinations thereof.

In another aspect, the method for generating an amino acid sequence directed against the first and second naturally occurring binding molecule may comprise at least the steps of:
a) providing a collection or sample of cells expressing amino acid sequences and/or Nanobodies;
b) screening said collection or sample of cells for cells that express an amino acid sequence and/or Nanobody that can bind to and/or has affinity for the first naturally occurring binding molecule;
c) screening said collection or sample of cells for cells that express an amino acid sequence and/or Nanobody that can bind to and/or has affinity for the second naturally occurring binding molecule;
   and
d) from the cell that expresses an amino acid sequence and/or Nanobody that can bind to and/or have affinity for the first and second naturally occurring binding molecule either (i) isolating said amino acid sequence and/or Nanobody; or (ii) isolating from said cell a nucleic acid sequence that encodes said amino acid sequence and/or Nanobody, followed by expressing said amino acid sequence and/or Nanobody.

In particular, such a method can comprise the steps of:
a) providing a collection or sample of cells expressing amino acid sequences and/or Nanobodies;
b) screening said collection or sample of cells for cells that express an amino acid sequence and/or Nanobody that can bind to and/or has affinity for the first naturally occurring binding molecule;
c) screening said cells obtained in b) for cells that express an amino acid sequence and/or Nanobody that can bind to and/or has affinity for the second naturally occurring binding molecule;
d) from the cell that expresses an amino acid sequence and/or Nanobody that can bind to and/or has affinity for the first and second naturally occurring binding molecule either (i) isolating said amino acid sequence and/or Nanobody; or (ii) isolating from said cell a nucleic acid sequence that encodes said amino acid sequence and/or Nanobody, followed by expressing said amino acid sequence.

The screening process can be easily performed comprising, for example the following steps:
a) providing a collection or sample of cells from a Camelid immunized with the first and second naturally occurring binding molecule;
b) from this collection or sample, separating cells that express antibodies from cells that do not express antibodies;
c) from the cells obtained in b), separating cells that express antibodies against the first naturally occurring binding molecule from cells that express antibodies directed against other antigens;
d) from the cells obtained in c), separating cells that express antibodies against the second naturally occurring binding molecule from cells that do not express such antibodies
e) from the cells obtained in d), separating cells that express heavy chain antibodies from cells that express conventional 4-chain antibodies;
f) obtaining from the cells obtained in e) a nucleic acid or nucleotide sequence that encodes a heavy chain antibody directed against the first and second naturally occurring binding molecule or that encodes an antigen-binding fragment thereof directed against said first and second naturally occurring binding molecule;
   in which steps b) to e) can be performed in any order.

In a preferred aspect of the invention, the collection or sample of cells expressing amino acid sequences is screened for amino acid sequences that can bind to the first naturally occurring binding molecule with a dissociation constant (K_{D}), a kₒₙ-rate and/or a k_{off}-rate that approximates the dissociation constant (K_{D}), kₒₙ-rate and/or k_{off}-rate with which said amino acid sequence can bind to the second naturally occurring binding molecule. In another preferred aspect of the invention, the collection or sample of cells expressing amino acid sequences is screened for amino acid sequences that can bind to the first naturally occurring binding molecule with a dissociation constant (K_{D}), a kₒₙ-rate and/or a k_{off}-rate that is at least 2 fold more, at least 5 fold more, at least 10 fold more, preferably at least 100 fold more, more preferably at least 1000 fold more, than the dissociation constant (K_{D}), kₒₙ-rate and/or k_{off}-rate with which said amino acid sequence can bind to the second naturally occurring binding molecule. In another preferred aspect of the invention, the collection or sample of cells expressing amino acid sequences is screened for amino acid sequences that can bind to the second naturally occurring binding molecule with a dissociation constant (K_{D}), a kₒₙ-rate and/or a k_{off}-rate that is at least 2 fold more, at least 5 fold more, at least 10 fold more, preferably at least 100 fold more, more preferably at least 1000 fold more, than the dissociation constant (K_{D}), kᵣₙ-rate and/or k_{off}-rate with which said amino acid sequence can bind to the first naturally occurring binding molecule.

In another preferred aspect of the invention, the collection or sample of cells expressing amino acid sequences is screened for amino acid sequences that can bind to the first naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said first naturally occurring binding molecule. In another preferred aspect of the invention, the collection or sample of cells expressing amino acid sequences is screened for amino acid sequences that can bind to said second naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to the second naturally occurring binding molecule. In another preferred aspect of the invention, the collection or sample of cells expressing amino acid sequences is screened for amino acid sequences that can bind to the first naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said first naturally occurring binding molecule and that can bind to the second naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said second naturally occurring binding molecule.

For example, when the desired amino acid sequence is an immunoglobulin sequence, the collection or sample of cells may for example be a collection or sample of B-cells. Also, in this method, the sample of cells may be derived from a mammal that has been suitably immunized with the first and/or second naturally occurring binding molecule or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, 97: 3820 (2001). Particular reference is made to the so-called “Nanoclone®” technique described in International application WO 06/079372 by Ablynx N.V.

In another aspect, the method for generating an amino acid sequence directed against the first and second naturally occurring binding molecule may comprise at least the steps of:
a) providing a set, collection or library of nucleic acid sequences encoding amino acid sequences and/or Nanobodies;
b) screening said set, collection or library of nucleic acid sequences for nucleic acid sequences that encode an amino acid sequence and/or Nanobody that can bind to and/or has affinity for the first naturally occurring binding molecule;
c) screening said set, collection or library of nucleic acid sequences for nucleic acid sequences that encode an amino acid sequence and/or Nanobody that can bind to and/or has affinity for the second naturally occurring binding molecule;
   and
d) isolating said nucleic acid sequence that encode an amino acid sequence and/or Nanobody that can bind to and/or has affinity for the first and second naturally occurring binding molecule, followed by expressing said amino acid sequence and/or Nanobody.

In particular, such a method can comprise the steps of:
a) providing a set, collection or library of nucleic acid sequences encoding amino acid sequences and/or Nanobody;
b) screening said set, collection or library of nucleic acid sequences for nucleic acid sequences that encode an amino acid sequence and/or Nanobody that can bind to and/or has affinity for the first naturally occurring binding molecule;
c) screening said nucleic acid sequences obtained in b) for nucleic acid sequences that encode an amino acid sequence and/or Nanobody that can bind to and/or has affinity for the second naturally occurring binding molecule;
   and
d) isolating said nucleic acid sequence that encode an amino acid sequence and/or Nanobody that can bind to and/or has affinity for the first and second naturally occurring binding molecule, followed by expressing said amino acid sequence.

If desired, an additional isolation step can be included between the two screening steps, isolating the nucleic acid sequence(s) that encode a amino acid sequence(s) that can bind to and/or have affinity for said first naturally occurring binding molecule.

In a preferred aspect of the invention, the set, collection or library of nucleic acid sequences is screened for nucleic acid sequences that encode an amino acid sequence that can bind to the first naturally occurring binding molecule with a dissociation constant (K_{D}), a kₒₙ-rate and/or a k_{off}-rate that approximates the dissociation constant (K_{D}), kₒₙ-rate and/or k_{off}-rate with which said amino acid sequence can bind to the second naturally occurring binding molecule. In another preferred aspect of the invention, the set, collection or library of nucleic acid sequences is screened for nucleic acid sequences that encode an amino acid sequence that can bind to the first naturally occurring binding molecule with a dissociation constant (K_{D}), a kₒₙ-rate and/or a k_{off}-rate that is at least 2 fold more, at least 5 fold more, at least 10 fold more, preferably at least 100 fold more, more preferably at least 1000 fold more, than the dissociation constant (K_{D}), kₒₙ-rate and/or k_{off}-rate with which said amino acid sequence can bind to the second naturally occurring binding molecule. In another preferred aspect of the invention, the set, collection or library of nucleic acid sequences is screened for nucleic acid sequences that encode an amino acid sequence that can bind to the second naturally occurring binding molecule with a dissociation constant (K_{D}), a kₒₙ-rate and/or a k_{off}-rate that is at least 2 fold more, at least 5 fold more, at least 10 fold more, preferably at least 100 fold more, more preferably at least 1000 fold more, than the dissociation constant (K_{D}), kₒₙ-rate and/or k_{off}-rate with which said amino acid sequence can bind to the first naturally occurring binding molecule.

In another preferred aspect of the invention, the set, collection or library of nucleic acid sequences is screened for nucleic acid sequences that encode an amino acid sequence that can bind to the first naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said first naturally occurring binding molecule. In another preferred aspect of the invention, the set, collection or library of nucleic acid sequences is screened for nucleic acid sequences that encode an amino acid sequence that can bind to the second naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said second naturally occurring binding molecule. In another preferred aspect of the invention, the set, collection or library of nucleic acid sequences is screened for nucleic acid sequences that encode an amino acid sequence that can bind to the first naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said first naturally occurring binding molecule and that can bind to the second naturally occurring binding molecule with a dissociation constant (K_{D}), a kₒₙ-rate and/or a k_{off}-rate that approximates (or that is lower or higher than) the dissociation constant (K_{D}), kₒₙ-rate and/or k_{off}-rate with which the third naturally occurring binding molecule can bind to said second naturally occurring binding molecule.

In such a method, the set, collection or library of nucleic acid sequences encoding amino acid sequences may for example be a set, collection or library of nucleic acid sequences encoding a naïve set, collection or library of immunoglobulin sequences; a set, collection or library of nucleic acid sequences encoding a synthetic or semi-synthetic set, collection or library of immunoglobulin sequences; and/or a set, collection or library of nucleic acid sequences encoding a set, collection or library of immunoglobulin sequences that have been subjected to affinity maturation.

Also, in such a method, the set, collection or library of nucleic acid sequences may encode a set, collection or library of heavy chain variable domains (such as V_{H} domains or V_{HH} domains) or of light chain variable domains. For example, the set, collection or library of nucleic acid sequences may encode a set, collection or library of domain antibodies or single domain antibodies, or a set, collection or library of amino acid sequences that are capable of functioning as a domain antibody or single domain antibody.

In a preferred aspect of this method. the set, collection or library of amino acid sequences may be an immune set, collection or library of nucleic acid sequences, for example derived from a mammal that has been suitably immunized with the first and/or second naturally occurring binding molecule or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The set, collection or library of nucleic acid sequences may for example encode an immune set, collection or library of heavy chain variable domains or of light chain variable domains. In one specific aspect, the set, collection or library of nucleotide sequences may encode a set, collection or library of sequences.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to WO 03/054013 and to the review by Hoogenboom in Nature Biotechnology, 23, 9, 1105-1116 (2005).

As will be clear to the skilled person, the screening step of the methods described herein can also be performed as a selection step. Accordingly the term “screening” as used in the present description can comprise selection, screening or any suitable combination of selection and/or screening techniques. Also, when a set, collection or library of sequences is used, it may contain any suitable number of sequences, such as 1, 2, 3 or about 5, 10, 50, 100, 500, 1000, 5000, 10⁴, 10⁵, 10⁶, 10⁷, 10⁸ or more sequences.

The above set, collection or library of nucleotide sequences may contain one or more sequences that encode an amino acid sequence that is not known in advance of the selection and or screening process for example if these sequences are the result of a randomization step (e.g. via error-prone PCR or other means) of one or more given nucleotide sequences. Also, one or more or all of the nucleotide sequences in the above set, collection or library nucleotide sequences may be obtained or defined by rational, or semi-empirical approaches such as computer modelling techniques or biostatics or data-mining techniques wherein amino acid sequences encode by these nucleotide sequences may have been defined or proposed that are predicted or expected to be endowed with certain properties such as increased stability, pH optimum, protease sensitivity or other properties or combinations thereof.

Yet another technique for obtaining amino acid sequences or Nanobody sequences directed against a first and a second naturally occurring binding molecule involves suitably immunizing a transgenic mammal that is capable of expressing heavy chain antibodies (i.e. so as to raise an immune response and/or heavy chain antibodies directed against the first and second naturally occurring binding molecule), obtaining a suitable biological sample from said transgenic mammal that contains (nucleic acid sequences encoding) said amino acid sequences or Nanobody sequences (such as a blood sample, serum sample or sample of B-cells), and then generating amino acid sequences directed against the first and the second naturally occurring binding molecules, starting from said sample, using any suitable technique known per se (such as any of the methods described herein or a hybridoma technique). For example, for this purpose, the heavy chain antibody-expressing mice and the further methods and techniques described in WO 02/085945, WO 04/049794 and WO 06/008548 and Janssens et al. (Proc. Natl. Acad. Sci. USA 103: 15130-5, 2006 can be used.

The invention also relates to amino acid sequences that are obtainable or obtained by the above methods, or alternatively by a method that comprises the one of the above methods and in addition at least the steps of determining the nucleotide sequence or amino acid sequence of said amino acid sequence; and of expressing or synthesizing said amino acid sequence in a manner known per se, such as by expression in a suitable host cell or host organism or by chemical synthesis.

In particular, the invention also relates to the V_{HH} sequences or Nanobody sequences that are obtained by the above methods, or alternatively by a method that comprises the one of the above methods and in addition at least the steps of determining the nucleotide sequence or amino acid sequence of said V_{HH} sequence or Nanobody sequence; and of expressing or synthesizing said V_{HH} sequence or Nanobody sequence in a manner known per se, such as by expression in a suitable host cell or host organism or by chemical synthesis.

As mentioned herein, a particularly preferred class of Nanobodies of the invention comprises Nanobodies with an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art on humanization referred to herein. Again, it should be noted that such humanized Nanobodies of the invention can be obtained in any suitable manner known per se (i.e. as indicated under points (1)-(8) above) and thus are not strictly limited to polypeptides that have been obtained using a polypeptide that comprises a naturally occurring V_{HH} domain as a starting material.

Another particularly preferred class of Nanobodies of the invention comprises Nanobodies with an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein. Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized Nanobody is preferably a V_{H} sequence from a mammal, more preferably the V_{H} sequence of a human being, such as a V_{H}3 sequence. However, it should be noted that such camelized Nanobodies of the invention can be obtained in any suitable manner known per se (i.e. as indicated under points (1)-(8) above) and thus are not strictly limited to polypeptides that have been obtained using a polypeptide that comprises a naturally occurring V_{H} domain as a starting material.

For example, again as further described herein, both “humanization” and “camelization” can be performed by providing a nucleotide sequence that encodes a naturally occurring V_{HH} domain or V_{H} domain, respectively, and then changing, in a manner known per se, one or more codons in said nucleotide sequence in such a way that the new nucleotide sequence encodes a “humanized” or “camelized” Nanobody of the invention, respectively. This nucleic acid can then be expressed in a manner known per se, so as to provide the desired Nanobody of the invention. Alternatively, based on the amino acid sequence of a naturally occurring V_{HH} domain or V_{H} domain, respectively, the amino acid sequence of the desired humanized or camelized Nanobody of the invention, respectively, can be designed and then synthesized de novo using techniques for peptide synthesis known per se. Also, based on the amino acid sequence or nucleotide sequence of a naturally occurring domain or V_{H} domain, respectively, a nucleotide sequence encoding the desired humanized or camelized Nanobody of the invention, respectively, can be designed and then synthesized de novo using techniques for nucleic acid synthesis known per se, after which the nucleic acid thus obtained can be expressed in a manner known per se, so as to provide the desired Nanobody of the invention.

Other suitable methods and techniques for obtaining the Nanobodies of the invention and/or nucleic acids encoding the same, starting from naturally occurring V_{H} sequences or preferably V_{HH} sequences, will be clear from the skilled person, and may for example comprise combining one or more parts of one or more naturally occurring V_{H} sequences (such as one or more FR sequences and/or CDR sequences), one or more parts of one or more naturally occurring V_{HH} sequences (such as one or more FR sequences or CDR sequences), and/or one or more synthetic or semi-synthetic sequences, in a suitable manner, so as to provide a Nanobody of the invention or a nucleotide sequence or nucleic acid encoding the same (which may then be suitably expressed). Nucleotide sequences encoding framework sequences of V_{HH} sequences or Nanobodies will be clear to the skilled person based on the disclosure herein and/or the further prior art cited herein (and/or may alternatively be obtained by PCR starting from the nucleotide sequences obtained using the methods described herein) and may be suitably combined with nucleotide sequences that encode the desired. CDR's (for example, by PCR assembly using overlapping primers), so as to provide a nucleic acid encoding a Nanobody of the invention.

As will be clear from the disclosure herein, it is also within the scope of the invention to use natural or synthetic analogs, mutants, variants, alleles, homologs and orthologs (herein collectively referred to as “analogs”) of the binders, amino acid sequences or Nanobodies of the invention as defined herein, and in particular analogs of the Nanobodies of SEQ ID NO's 22-24. Thus, according to one aspect of the invention, the term “Nanobody of the invention” in its broadest sense also covers such analogs.

Generally, in such analogs, one or more amino acid residues may have been replaced, deleted and/or added, compared to the binders, amino acid sequences or Nanobodies of the invention as defined herein. Such substitutions, insertions or deletions may be made in one or more of the framework regions and/or in one or more of the CDR's. When such substitutions, insertions or deletions are made in one or more of the framework regions, they may be made at one or more of the Hallmark residues and/or at one or more of the other positions in the framework residues, although substitutions, insertions or deletions at the Hallmark residues are generally less preferred (unless these are suitable humanizing substitutions as described herein).

As will also be clear from the disclosure herein, it is also within the scope of the invention to use parts or fragments, or combinations of two or more parts or fragments, of the binders, amino acid sequences and/or Nanobodies of the invention as defined herein, and in particular parts or fragments of the Nanobodies of SEQ ID NO's: 22-24. Thus, according to one aspect of the invention, the term “Nanobody of the invention” in its broadest sense also covers such parts or fragments.

Generally, such parts or fragments of the binders, amino acid sequences and/or Nanobodies of the invention (including analogs thereof) have amino acid sequences in which, compared to the amino acid sequence of the corresponding full length binder, amino acid sequence or Nanobody of the invention (or analog thereof), one or more of the amino acid residues at the N-terminal end, one or more amino acid residues at the C-terminal end, one or more contiguous internal amino acid residues, or any combination thereof, have been deleted and/or removed.

The parts or fragments are preferably such that they can bind to the at least two naturally occurring binding molecules with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the binders, amino acid sequences and Nanobodies of the invention.

Any part or fragment of an amino acid or Nanobody of the invention is preferably such that it comprises at least 10 contiguous amino acid residues, preferably at least 20 contiguous amino acid residues, more preferably at least 30 contiguous amino acid residues, such as at least 40 contiguous amino acid residues, of the amino acid sequence of the corresponding full length amino acid sequence or Nanobody of the invention.

Also, any part or fragment is such preferably that it comprises at least one of CDR1, CDR2 and/or CDR3 or at least part thereof (and in particular at least CDR3 or at least part thereof). More preferably, any part or fragment is such that it comprises at least one of the CDR's (and preferably at least CDR3 or part thereof) and at least one other CDR (i.e. CDR1 or CDR2) or at least part thereof, preferably connected by suitable framework sequence(s) or at least part thereof. More preferably, any part or fragment is such that it comprises at least one of the CDR's (and preferably at least CDR3 or part thereof) and at least part of the two remaining CDR's, again preferably connected by suitable framework sequence(s) or at least part thereof.

According to another particularly preferred, but non-limiting aspect, such a part or fragment comprises at least CDR3, such as FR3, CDR3 and FR4 of the corresponding full length Nanobody of the invention, i.e. as for example described in the International application WO 03/050531 (Lasters et al.).

As already mentioned above, it is also possible to combine two or more of such parts or fragments (i.e. from the same or different binders, amino acid sequences and/or Nanobodies of the invention), i.e. to provide an analog (as defined herein) and/or to provide further parts or fragments (as defined herein) of a binder, amino acid sequence or Nanobody of the invention. It is for example also possible to combine one or more parts or fragments a Nanobody of the invention with one or more parts or fragments of a human V_{H} domain.

According to one preferred aspect, the parts or fragments have a degree of sequence identity of at least 50%, preferably at least 60%, more preferably at least 70%, even more preferably at least 80%, such as at least 90%, 95% or 99% or more with one of the Nanobodies of SEQ ID NOs 22-24.

The parts and fragments, and nucleic acid sequences encoding the same, can be provided and optionally combined in any manner known per se. For example, such parts or fragments can be obtained by inserting a stop codon in a nucleic acid that encodes a full-sized amino acid or Nanobody of the invention, and then expressing the nucleic acid thus obtained in a manner known per se (e.g. as described herein). Alternatively, nucleic acids encoding such parts or fragments can be obtained by suitably restricting a nucleic acid that encodes a full-sized amino acid or Nanobody of the invention or by synthesizing such a nucleic acid in a manner known per se. Parts or fragments may also be provided using techniques for peptide synthesis known per se.

The invention in its broadest sense also comprises derivatives of the binders, amino acid sequences and Nanobodies of the invention. Such derivatives can generally be obtained by modification, and in particular by chemical and/or biological (e.g enzymatical) modification, of the binders, amino acid sequences or Nanobodies of the invention.

Examples of such modifications, as well as examples of amino acid residues within the Nanobody sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the binder, amino acid sequence or Nanobody of the invention, and in particular of one or more functional groups, residues or moieties that confer one or more desired properties or functionalities to the binder, amino acid sequence or Nanobody of the invention. Example of such functional groups will be clear to the skilled person.

For example, such modification may comprise the introduction (e.g. by covalent binding or in any other suitable manner) of one or more functional groups that increase the half-life, the solubility and/or the absorption of the binder, amino acid sequence or Nanobody of the invention, that reduce the immunogenicity and/or the toxicity of the binder, amino acid sequence or Nanobody and/or polypeptide of the invention, that eliminate or attenuate any undesirable side effects of the binder, amino acid sequence or Nanobody and/or polypeptide of the invention, and/or that confer other advantageous properties to and/or reduce the undesired properties of the binder, amino acid sequence or Nanobodies and/or polypeptides of the invention; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFv's and single domain antibodies), for which reference is for example made to Remington's Pharmaceutical Sciences, 16th ed., Mack Publishing Co., Easton, Pa. (1980). Such functional groups may for example be linked directly (for example covalently) to a binder, amino acid sequence or Nanobody of the invention, or optionally via a suitable linker or spacer, as will again be clear to the skilled person.

One of the most widely used techniques for increasing the half-life and/or reducing the immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFv's); reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see for example Yang et al., Protein Engineering, 16, 10, 761-770 (2003). For example, for this purpose, PEG may be attached to a cysteine residue that naturally occurs in an amino acid sequence or Nanobody of the invention, an amino acid sequence or Nanobody of the invention may be modified so as to suitably introduce one or more cysteine residues for attachment of PEG, or an amino acid sequence comprising one or more cysteine residues for attachment of PEG may be fused to the N- and/or C-terminus of an amino acid sequence or Nanobody of the invention, all using techniques of protein engineering known per se to the skilled person.

Preferably, for the amino acid sequences, Nanobodies and polypeptides of the invention, a PEG is used with a molecular weight of more than 5000, such as more than 10,000 and less than 200,000, such as less than 100,000; for example in the range of 20,000-80,000.

Another, usually less preferred modification comprises N-linked or O-linked glycosylation, usually as part of co-translational and/or post-translational modification, depending on the host cell used for expressing the amino acid sequence, Nanobody or polypeptide of the invention.

Yet another modification may comprise the introduction of one or more detectable labels or other signal-generating groups or moieties, depending on the intended use of the labelled binder, amino acid sequence or Nanobody. Suitable labels and techniques for attaching, using and detecting them will be clear to the skilled person, and for example include, but are not limited to, fluorescent labels (such as fluorescein, isothiocyanate, rhodamine, phycoerythrin, phycocyanin, allophycocyanin, o-phthaldehyde, and fluorescamine and fluorescent metals such as ¹⁵²Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes (such as ³H, ¹²⁵I, ³²P, ³⁵S, ¹⁴C, ⁵¹Cr, ³⁶Cl, ⁵⁷Co, ⁵⁸Co, ⁵⁹Fe, and ⁷⁵Se), metals, metal chelates or metallic cations (for example metallic cations such as ^{99m}Tc, ¹²³I, ¹¹¹In, ¹³¹I, ⁹⁷Ru, ⁶⁷Cu, ⁶⁷Ga, and ⁶⁸Ga or other metals or metallic cations that are particularly suited for use in in vivo, in vitro or in situ diagnosis and imaging, such as (¹⁵⁷Gd, ⁵⁵Mn, ¹⁶²Dy, ⁵²Cr, and ⁵⁶Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy.

Such labelled binders, amino acid sequences, Nanobodies, compounds and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethyl-enetriaminepentaacetic acid (DTPA) or ethylenediaminetetraacetic acid (EDTA).

Yet another modification may comprise the introduction of a functional group that is one part of a specific binding pair, such as the biotin-(strept)avidin binding pair. Such a functional group may be used to link the binder, amino acid sequence and/or Nanobody of the invention to another protein, polypeptide or chemical compound that is bound to the other half of the binding pair, i.e. through formation of the binding pair. For example, a binder, amino acid sequence or Nanobody of the invention may be conjugated to biotin, and linked to another protein, polypeptide, compound or carrier conjugated to avidin or streptavidin. For example, such a conjugated binder, amino acid sequence or Nanobody may be used as a reporter, for example in a diagnostic system where a detectable signal-producing agent is conjugated to avidin or streptavidin. Such binding pairs may for example also be used to bind the binder, amino acid sequence or Nanobody of the invention to a carrier, including carriers suitable for pharmaceutical purposes. One non-limiting example are the liposomal formulations described by Cao and Suresh, Journal of Drug Targetting, 8, 4, 257 (2000). Such binding pairs may also be used to link a therapeutically active agent to the binder, amino acid sequence or Nanobody of the invention.

For some applications, in particular for those applications in which it is intended to kill a cell that expresses the target against which the binder, amino acid sequence or Nanobodies of the invention are directed (e.g. in the treatment of cancer), or to reduce or slow the growth and/or proliferation such a cell, the binders, amino acid sequences or Nanobodies of the invention may also be linked to a toxin or to a toxic residue or moiety. Examples of toxic moieties, compounds or residues which can be linked to a binder, amino acid sequence or Nanobody of the invention to provide—for example—a cytotoxic compound will be clear to the skilled person and can for example be found in the prior art cited above and/or in the further description herein. One example is the so-called ADEPT™ technology described in WO 03/055527.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

Preferably, the derivatives are such that they bind to the at least two naturally occurring binding molecules with an affinity (suitably measured and/or expressed as a Ku-value (actual or apparent), a K_{A}-value (actual or apparent), a kₒₙ-rate and/or a k_{off}-rate, or alternatively as an IC₅₀ value, as further described herein) that is as defined herein for the binders, amino acid sequences and Nanobodies of the invention.

The amino acid sequences, Nanobodies, polypeptides and nucleic acids of the invention can be prepared in a manner known per se, as will be clear to the skilled person from the further description herein. For example, the Nanobodies and polypeptides of the invention can be prepared in any manner known per se for the preparation of antibodies and in particular for the preparation of antibody fragments (including but not limited to (single) domain antibodies and ScFv fragments). Some preferred, but non-limiting methods for preparing the amino acid sequences, Nanobodies, polypeptides and nucleic acids include the methods and techniques described herein.

As will be clear to the skilled person, one particularly useful method for preparing an amino acid sequence, Nanobody and/or a polypeptide of the invention generally comprises the steps of:
i) the expression, in a suitable host cell or host organism (also referred to herein as a “host of the invention”) or in another suitable expression system of a nucleic acid that encodes said amino acid sequence, Nanobody or polypeptide of the invention (also referred to herein as a “nucleic acid of the invention”), optionally followed by:
ii) isolating and/or purifying the amino acid sequence. Nanobody or polypeptide of the invention thus obtained.

In particular, such a method may comprise the steps of:
i) cultivating and/or maintaining a host of the invention under conditions that are such that said host of the invention expresses and/or produces at least one amino acid sequence, Nanobody and/or polypeptide of the invention; optionally followed by:
ii) isolating and/or purifying the amino acid sequence, Nanobody or polypeptide of the invention thus obtained.

A nucleic acid of the invention can be in the form of single or double stranded. DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one aspect of the invention, the nucleic acid of the invention is in essentially isolated from, as defined herein.

The nucleic acid of the invention may also be in the form of, be present in and/or be part of a vector, such as for example a plasmid. cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring form of the nucleic acid as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting aspect, a genetic construct of the invention comprises
i) at least one nucleic acid of the invention; operably connected to
ii) one or more regulatory elements, such as a promoter and optionally a suitable terminator;
   and optionally also
iii) one or more further elements of genetic constructs known per se; in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used. For example, regulatory requences, promoters and terminators known per se for the expression and production of antibodies and antibody fragments (including but not limited to (single) domain antibodies and ScFv fragments) may be used in an essentially analogous manner.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promoter). Generally, when two nucleotide sequences are operably linked, they will be in the same orientation and usually also in the same reading frame. They will usually also be essentially contiguous, although this may also not be required.

Preferably, the regulatory and further elements of the genetic constructs of the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined herein).

Some particularly preferred promoters include, but are not limited to, promoters known per se for the expression in the host cells mentioned herein; and in particular promoters for the expression in the bacterial cells, such as those mentioned herein and/or those used in the Examples.

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. Leader sequences may not be required for expression in a bacterial cell. For example, leader sequences known per se for the expression and production of antibodies and antibody fragments (including but not limited to single domain antibodies and ScFv fragments) may be used in an essentially analogous manner.

An expression marker or reporter gene should be such that—in the host cell or host organism—it allows for detection of the expression of (a gene or nucleotide sequence present on) the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as GFP.

Some preferred, but non-limiting examples of suitable promoters, terminator and further elements include those that can be used for the expression in the host cells mentioned herein; and in particular those that are suitable for expression in bacterial cells, such as those mentioned herein and/or those used in the Examples below. For some (further) non-limiting examples of the promoters, selection markers, leader sequences, expression markers and further elements that may be present/used in the genetic constructs of the invention—such as terminators, transcriptional and/or translational enhancers and/or integration factors—reference is made to the general handbooks such as Sambrook et al. and Ausubel et al. mentioned above, as well as to the examples that are given in WO 95/07463, WO 96/23810, WO 95/07463, WO 95/21191, WO 97/11094, WO 97/42320, WO 98/06737, WO 98/21355, U.S. Pat. No. 7,207,410, U.S. Pat. No. 5,693,492 and EP 1 085 089. Other examples will be clear to the skilled person. Reference is also made to the general background art cited above and the further references cited herein.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors are those used in the Examples below, as well as those mentioned herein.

The nucleic acids of the invention and/or the genetic constructs of the invention may be used to transform a host cell or host organism, i.e. for expression and/or production of the amino acid sequence, Nanobody or polypeptide of the invention. Suitable hosts or host cells will be clear to the skilled person, and may for example be any suitable fungal, prokaryotic or eukaryotic cell or cell line or any suitable fungal, prokaryotic or eukaryotic organism, for example:
a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis;
a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi;
a yeast cell, including but not limited to cells from species of Saccharomyces, for example of Saccharomyces cerevisiae; of Schizosaecharomyces, for example of Schizosaccharomyces pombe; of Pichia, for example of Pichia pastoris or of Pichia methanolica; of Hansenula, for example of Hansenula polymorphs; of Kluyveromyces, for example of Kluyveromyces lactis; of Arxula, for example of Arxula adeninivorans; of Yarrowia, for example of Yarrowia lipolytica;
an amphibian cell or cell line, such as Xenopus oocytes;
an insect-derived cell or cell line, such as cells/cell lines derived from lepidoptera, including but not limited to Spodoptera SF9 and Sf21 cells or cells/cell lines derived from Drosophila, such as Schneider and Kc cells;
a plant or plant cell, for example in tobacco plants; and/or
a mammalian cell or cell line, for example a cell or cell line derived from a human, a cell or a cell line from mammals including but not limited to CHO-cells, BHK-cells (for example BHK-21 cells) and human cells or cell lines such as HeLa, COS (for example COS-7) and PER.C6 cells;
   as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94129457; WO 96/34103; WO 99/42077; Frenken et al., (1998), supra; Rieehmann and Muyldermans, (1999), supra; van der Linden, (2000), supra; Thomassen et al., (2002), supra; Joosten et al., (2003), supra; Joosten et al., (2005), supra; and the further references cited herein.

The amino acid sequences, Nanobodies and polypeptides of the invention can also be introduced and expressed in one or more cells, tissues or organs of a multicellular organism, for example for prophylactic and/or therapeutic purposes (e.g. as a gene therapy). For this purpose, the nucleotide sequences of the invention may be introduced into the cells or tissues in any suitable way, for example as such (e.g. using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patient by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, and for example described in Culver, K. W., “Gene Therapy”, 1994, p. xii, Mary Ann Liebert, Inc., Publishers, New York, N.Y); Giordano, Nature F Medicine 2 (1996), 534-539; Schaper, Circ. Res. 79 (1996), 911-919; Anderson, Science 256 (1992),808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; U.S. Pat. No. 5,589,546; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

For expression of the Nanobodies in a cell, they may also be expressed as so-called “intrabodies”, as for example described in. WO 94/02610, WO 95/22618 and U.S. Pat. No. 7,004,940; WO 03/014960; in. Cattaneo, A. & Biocca, S. (1997) Intracellular Antibodies: Development and Applications. Landes and Springer-Verlag; and in Konterrnann, Methods 34, (2004), 163-170.

The amino acid sequences, Nanobodies and polypeptides of the invention can for example also be produced in the milk of transgenic mammals, for example in the milk of rabbits, cows, goats or sheep (see for example U.S. Pat. No. 6,741,957, U.S. Pat. No. 6,304,489 and U.S. Pat. No. 6,849,992 for general techniques for introducing transgenes into mammals), in plants or parts of plants including but not limited to their leaves, flowers, fruits, seed, roots or turbers (for example in tobacco, maize, soybean or alfalfa) or in for example pupae of the silkworm Bombix mori.

Furthermore, the amino acid sequences, Nanobodies and polypeptides of the invention can also be expressed and/or produced in cell-free expression systems, and suitable examples of such systems will be clear to the skilled person. Some preferred, but non-limiting examples include expression in the wheat germ system; in rabbit reticulocyte lysates; or in the E. coli Zubay system.

As mentioned above, one of the advantages of the use of Nanobodies is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, polypeptides of the invention suitable for pharmaceutical use can be prepared using techniques for peptide synthesis.

For production on industrial scale, preferred heterologous hosts for the (industrial) production of Nanobodies or Nanobody-containing protein therapeutics include strains of E. coli, Pichia pastoris, S. cerevisiae that are suitable for large scale expression/production/fermentation, and in particular for large scale pharmaceutical (i.e. GMP grade) expression/production/fermentation. Suitable examples of such strains will be clear to the skilled person. Such strains and production/expression systems are also made available by companies such as Biovitrum (Uppsala, Sweden).

Alternatively, mammalian cell lines, in particular Chinese hamster ovary (CHO) cells, can be used for large scale expression/production/fermentation, and in particular for large scale pharmaceutical expression/production/fermentation. Again, such expression/production systems are also made available by some of the companies mentioned above.

The choice of the specific expression system would depend in part on the requirement for certain post-translational modifications, more specifically glycosylation. The production of a Nanobody-containing recombinant protein for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression. Preferably, either a human cell or cell line is used (i.e. leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast usually leads to a glycosylation pattern that differs from human glycosylation. Nevertheless, it should be understood that all the foregoing host cells and expression systems can be used in the invention, depending on the desired amino acid sequence, Nanobody or polypeptide to be obtained.

Thus, according to one non-limiting aspect of the invention, the amino acid sequence, Nanobody or polypeptide of the invention is glycosylated. According to another non-limiting aspect of the invention, the amino acid sequence, Nanobody or polypeptide of the invention is non-glycosylated.

According to one preferred, but non-limiting aspect of the invention, the amino acid sequence, Nanobody or polypeptide of the invention is produced in a bacterial cell, in particular a bacterial cell suitable for large scale pharmaceutical production, such as cells of the strains mentioned above.

According to another preferred, but non-limiting aspect of the invention, the amino acid sequence, Nanobody or polypeptide of the invention is produced in a yeast cell, in particular a yeast cell suitable for large scale pharmaceutical production, such as cells of the species mentioned above.

According to yet another preferred, but non-limiting aspect of the invention, the amino acid sequence, Nanobody or polypeptide of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

When expression in a host cell is used to produce the amino acid sequences, Nanobodies and the polypeptides of the invention, the amino acid sequences, Nanobodies and polypeptides of the invention can be produced either intracellullarly (e.g. in the cytosol, in the periplasma or in inclusion bodies) and then isolated from the host cells and optionally further purified; or can be produced extracellularly (e.g. in the medium in which the host cells are cultured) and then isolated from the culture medium and optionally further purified. When eukaryotic host cells are used, extracellular production is usually preferred since this considerably facilitates the further isolation and downstream processing of the Nanobodies and proteins obtained. Bacterial cells such as the strains of E. coli mentioned above normally do not secrete proteins extracellularly, except for a few classes of proteins such as toxins and hemolysin, and secretory production in E. coli refers to the translocation of proteins across the inner membrane to the periplasmic space. Periplasmic production provides several advantages over cytosolic production. For example, the N-terminal amino acid sequence of the secreted product can be identical to the natural gene product after cleavage of the secretion signal sequence by a specific signal peptidase. Also, there appears to be much less protease activity in the periplasm than in the cytoplasm. In addition, protein purification is simpler due to fewer contaminating proteins in the periplasm. Another advantage is that correct disulfide bonds may form because the periplasm provides a more oxidative environment than the cytoplasm. Proteins overexpressed in. E. coli are often found in insoluble aggregates, so-called inclusion bodies. These inclusion bodies may be located in the cytosol or in the periplasm; the recovery of biologically active proteins from these inclusion bodies requires a denaturation/refolding process. Many recombinant proteins, including therapeutic proteins, are recovered from inclusion bodies. Alternatively, as will be clear to the skilled person, recombinant strains of bacteria that have been genetically modified so as to secrete a desired protein, and in particular an amino acid sequence, Nanobody or a polypeptide of the invention, can be used.

Thus, according to one non-limiting aspect of the invention, the amino acid sequence, Nanobody or polypeptide of the invention is an amino acid sequence, Nanobody or polypeptide that has been produced intracellularly and that has been isolated from the host cell, and in particular from a bacterial cell or from an inclusion body in a bacterial cell. According to another non-limiting aspect of the invention, the amino acid sequence, Nanobody or polypeptide of the invention is an amino acid sequence, Nanobody or polypeptide that has been produced extracellularly, and that has been isolated from the medium in which the host cell is cultivated.

Some preferred, but non-limiting promoters for use with these host cells include,
for expression in E. coli: lac promoter (and derivatives thereof such as the lacUV5 promoter); arabinose promoter; left-(PL) and rightward (PR) promoter of phage lambda; promoter of the trp operon; hybrid lac/trp promoters (tac and trc); T7-promoter (more specifically that of T7-phage gene 10) and other T-phage promoters; promoter of the Tn 10 tetracycline resistance gene; engineered variants of the above promoters that include one or more copies of an extraneous regulatory operator sequence;
for expression in S. cerevisiae: constitutive: ADH1 (alcohol dehydrogenase 1), ENO (enolase), CYC (cytochrome c iso-1), GAPDH (glyceraldehydes-3-phosphate dehydrogenase), PGK1 (phosphoglycerate kinase), PYK1 (pyruvate kinase); regulated: GAL1,10,7 (galactose metabolic enzymes), ADH2 (alcohol dehydrogenase 2), PHO5 (acid phosphatase), CUP1 (copper metallothionein); heterologous: CaMV (cauliflower mosaic virus 35S promoter);
for expression in. Pichia pastoris: the AOX1 promoter (alcohol oxidase I);
for expression in mammalian cells: human cytomegalovirus (hCMV) immediate early enhancer/promoter; human cytomegalovirus (hCMV) immediate early promoter variant that contains two tetracycline operator sequences such that the promoter can be regulated by the Tet repressor; Herpes Simplex Virus thymidine kinase (TK) promoter; Rous Sarcoma Virus long terminal repeat (RSV LTR) enhancer/promoter; elongation factor 1α (hEF-1α) promoter from human, chimpanzee, mouse or rat; the SV40 early promoter; HIV-1 long terminal repeat promoter; β-actin promoter;

Some preferred, but non-limiting vectors for use with these host cells include:
vectors for expression in mammalian cells: pMAMneo (Clontech), pcDNA3 (Invitrogen), pMClneo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565), as well as viral-based expression systems, such as those based on adenovirus;
vectors for expression in bacterial cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacII (Invitrogen) and other baculovirus vectors
vectors for expression in plants or plant cells: for example vectors based on cauliflower mosaic virus or tobacco mosaic virus, suitable strains of Agrobacterium, or Ti-plasmid based vectors.

Some preferred, but non-limiting secretory sequences for use with these host cells include:
for use in bacterial cells such as E. coli: PelB, Bla, OmpA, OmpC, OmpF, OmpT, StII, PhoA, PhoE, MalE, Lpp, LamB, and the like; TAT signal peptide, hemolysin C-terminal secretion signal;
for use in yeast: α-mating factor prepro-sequence, phosphatase (phot), invertase (Sue), etc.;
for use in mammalian cells: indigenous signal in case the target protein is of eukaryotic origin; murine Ig κ-chain V-J2-C signal peptide; etc.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions). an amino acid sequence, Nanobody or polypeptide of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence, Nanobody or polypeptide of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence, Nanobody or polypeptide of the invention may (first) be generated in an immature form (as mentioned above), which may then be subjected to post-translational modification, depending on the host cell/host organism used. Also, the amino acid sequence, Nanobody or polypeptide of the invention may be glycosylated, again depending on the host cell/host organism used.

The amino acid sequence, Nanobody or polypeptide of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence, Nanobody or polypeptide of the invention) and/or preparative immunological techniques (i.e. using antibodies against the amino acid sequence to be isolated).

Generally, for pharmaceutical use, the compounds, polypeptides, binders, amino acid sequences and Nanobodies of the invention may be formulated as a pharmaceutical preparation or compositions comprising at least one compound, polypeptide, binder, amino acid sequence or Nanobody of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one binder of the invention, at least one amino acid of the invention, at least one Nanobody of the invention, at least one compound of the invention or at least one polypeptide of the invention and at least one suitable carrier, diluent or excipient (i.e. suitable for pharmaceutical use), and optionally one or more further active substances.

Generally, the binders, amino acid sequences, Nanobodies, compounds and polypeptides of the invention can be formulated and administered in any suitable manner known per se, for which reference is for example made to the general background art cited above (and in particular to WO 04/041862, WO 04/041863, WO 04/041865 and WO 04/041867) as well as to the standard handbooks, such as Remington's Pharmaceutical Sciences, 18^{th} Ed., Mack Publishing Company, USA (1990) or Remington, the Science and Practice of Pharmacy, 21th Edition, Lippincott Williams and Wilkins (2005).

For example, the binders, amino acid sequences, Nanobodies, compounds and polypeptides of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, sterile water and aqueous buffers and solutions such as physiological phosphate-buffered saline, Ringer's solutions, dextrose solution, and Hank's solution; water oils; glycerol; ethanol; glycols such as propylene glycol or as well as mineral oils, animal oils and vegetable oils, for example peanut oil, soybean oil, as well as suitable mixtures thereof. Usually, aqueous solutions or suspensions will be preferred.

The binders, amino acid sequences, Nanobodies, compounds and polypeptides of the invention can also be administered using gene therapy methods of delivery. See, e.g., U.S. Pat. No. 5,399,346, which is incorporated by reference in its entirety. Using a gene therapy method of delivery, primary cells transfected with the gene encoding an amino acid sequence, Nanobody or polypeptide of the invention can additionally be transfected with tissue specific promoters to target specific organs, tissue, grafts, tumors, or cells and can additionally be transfected with signal and stabilization sequences for subcellularly localized expression.

Thus, the binders, amino acid sequences, Nanobodies, compounds and polypeptides of the invention may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, binders, the amino acid sequences, Nanobodies, compounds and polypeptides of the invention may be combined with one or more excipients and used in the form of ingestible tablets, buccal tablets, troches, capsules, elixirs, suspensions, syrups, wafers, and the like. Such compositions and preparations should contain at least 0.1% of the binder, amino acid sequence, compound, Nanobody or polypeptide of the invention. Their percentage in the compositions and preparations may, of course, be varied and may conveniently be between about 2 to about 60% of the weight of a given unit dosage form. The amount of the binder, amino acid sequence, Nanobody, compound or polypeptide of the invention in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the binders, amino acid sequences, Nanobodies, compounds and polypeptides of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, binders, the amino acid sequences, Nanobodies, compounds and polypeptides of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The binders, amino acid sequences, Nanobodies, compounds and polypeptides of the invention may also be administered intravenously or intraperitoneally by infusion or injection. Solutions of the binders, amino acid sequences, Nanobodies, compounds and polypeptides of the invention or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the binders, amino acid sequences, Nanobodies, compounds and polypeptides of the invention in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the binders, amino acid sequences, Nanobodies, compounds and polypeptides of the invention may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the binders, amino acid sequences, Nanobodies, compounds and polypeptides of the invention can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the binders, amino acid sequences, Nanobodies, compounds and polypeptides of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the binders, amino acid sequences, Nanobodies, compounds and polypeptides of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the binders, amino acid sequences, Nanobodies, compounds and polypeptides of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the binders, amino acid sequences, Nanobodies, compounds and polypeptides of the invention required for use in treatment will vary not only with the particular binder, amino acid sequence, Nanobody, compound or polypeptide selected but also with the route of administration, the nature of the condition being treated and the age and condition of the patient and will be ultimately at the discretion of the attendant physician or clinician. Also the dosage of the binders, amino acid sequences, Nanobodies, compounds and polypeptides of the invention varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

In another aspect, the invention relates to a method for the prevention and/or treatment of at least one disease or disorder associated with a first, second and/or third naturally occurring binding molecule, said method comprising administering, to a subject in need thereof, a pharmaceutically active amount of a binder of the invention, of an amino acid sequence of the invention, of a Nanobody of the invention, of a compound of the invention, of a polypeptide of the invention, and/or of a pharmaceutical composition comprising the same.

In a preferred aspect, the invention relates to a method for the prevention and/or treatment of at least one of cancer, inflammatory diseases or osteoporosis, said method comprising administering, to a subject in need thereof, a pharmaceutically active amount of a binder of the invention, of an amino acid sequence of the invention, of a Nanobody of the invention, of a compound of the invention, of a polypeptide of the invention, and/or of a pharmaceutical composition comprising the same.

In a preferred aspect the cancer to be treated is melanoma, a tumor, soft tissue sarcoma, skin cancer, drug-resistant bony sarcomas, leukemia.

In another preferred aspect, the inflammatory disease to be treated is Crohn's disease, rheumatoid arthritis, systemic lupus erythematosus, Sjogren's syndrome, lymphohistocytosis, myocarditis, multiple sclerosis, autoimmune encephalomeyeltitis, insulin-dependent diabetes mellitus, allergies, allograft rejection, xeno transplant rejection and/or graft versus host disease.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention relates to a method for the prevention and/or treatment of at least one disease or disorder that is associated with a first, second and/or third naturally occurring binding molecule, with its biological or pharmacological activity, and/or with the biological pathways or signalling in which a first, second and/or third naturally occurring binding molecule is involved, said method comprising administering, to a subject in need thereof, a pharmaceutically active amount of a binder of the invention, of an amino acid sequence of the invention, of a Nanobody of the invention, of a compound of the invention, of a polypeptide of the invention, and/or of a pharmaceutical composition comprising the same. In particular, the invention relates to a method for the prevention and/or treatment of at least one disease or disorder that can be treated by modulating a first, second and/or third naturally occurring binding molecule, its biological or pharmacological activity, and/or the biological pathways or signalling in which a first, second and/or third naturally occurring binding molecule is involved, said method comprising administering, to a subject in need thereof, a pharmaceutically active amount of a binder of the invention, an amino acid sequence of the invention, of a Nanobody of the invention, of a compound of the invention, of a polypeptide of the invention, and/or of a pharmaceutical composition comprising the same. In particular, said pharmaceutically effective amount may be an amount that is sufficient to modulate a first, second and/or third naturally occurring binding molecule, its biological or pharmacological activity, and/or the biological pathways or signalling in which a first, second and/or third naturally occurring binding molecule is involved; and/or an amount that provides a level of a binder of the invention, an amino acid sequence of the invention, of a Nanobody of the invention, of a compound of the invention, of a polypeptide of the invention in the circulation that is sufficient to modulate a first, second and/or third naturally occurring binding molecule, its biological or pharmacological activity, and/or the biological pathways or signalling in which said first, second and/or third naturally occurring binding molecule is involved.

The invention furthermore relates to a method for the prevention and/or treatment of at least one disease or disorder that can be prevented and/or treated by administering a binder of the invention, an amino acid sequence of the invention, a Nanobody of the invention, a compound of the invention or a polypeptide of the invention to a patient, said method comprising administering, to a subject in need thereof, a pharmaceutically active amount of a binder of the invention, of an amino acid sequence of the invention, of a Nanobody of the invention, of a compound of the invention, of a polypeptide of the invention, and/or of a pharmaceutical composition comprising the same.

More in particular, the invention relates to a method for the prevention and/or treatment of at least one disease or disorder chosen from the group consisting of the diseases and disorders listed herein, said method comprising administering, to a subject in need thereof, a pharmaceutically active amount of a binder of the invention, of an amino acid sequence of the invention, of a Nanobody of the invention, of a compound of the invention, of a polypeptide of the invention, and/or of a pharmaceutical composition comprising the same.

In another aspect, the invention relates to a method for immunotherapy, and in particular for passive immunotherapy, which method comprises administering, to a subject suffering from or at risk of the diseases and disorders mentioned herein, a pharmaceutically active amount of a binder of the invention, of an amino acid sequence of the invention, of a Nanobody of the invention, of a compound of the invention, of a polypeptide of the invention, and/or of a pharmaceutical composition comprising the same.

In the above methods, the binders, amino acid sequences, Nanobodies, compounds and/or polypeptides of the invention and/or the compositions comprising the same can be administered in any suitable manner, depending on the specific pharmaceutical formulation or composition to be used. Thus, the binders, amino acid sequences, Nanobodies, compounds and/or polypeptides of the invention and/or the compositions comprising the same can for example be administered orally, intraperitoneally (e.g. intravenously, subcutaneously, intramuscularly, or via any other route of administration that circumvents the gastrointestinal tract), intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

The binders, amino acid sequences, Nanobodies, compounds and/or polypeptides of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific binder, amino acid sequence, Nanobody, compound or polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more binders, amino acid sequences, Nanobodies, compounds and/or polypeptides of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency of the specific binder, amino acid sequence, Nanobody, compound and polypeptide of the invention to be used, the specific route of administration and the specific pharmaceutical formulation or composition used, the binders, amino acid sequences, Nanobodies and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single binder, amino acid sequence, compound, Nanobody or polypeptide of the invention will be used. It is however within the scope of the invention to use two or more binders, amino acid sequences, Nanobodies, compounds and/or polypeptides of the invention in combination.

The binders, amino acid sequences, Nanobodies, compounds and polypeptides of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement.

In particular, the binders, amino acid sequences. Nanobodies, compounds and polypeptides of the invention may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and on a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In another aspect, the invention relates to the use of a binder, amino acid sequence, Nanobody, compound or polypeptide of the invention in the preparation of a pharmaceutical composition for prevention and/or treatment of at least one of cancer, inflammatory diseases or osteoporosis; and/or for use in one or more of the methods of treatment mentioned herein.

A binder, amino acid sequence, Nanobody, compound or polypeptide of the invention for prevention and/or treatment of at least one of cancer, inflammatory diseases or osteoporosis; and/or for use in one or more of the methods of treatment mentioned herein.

In a preferred aspect the cancer to be treated is melanoma, a tumor, soft tissue sarcoma, skin cancer, drug-resistant bony sarcomas, leukemia.

In another preferred aspect, the inflammatory disease to be treated is Crohn's disease, rheumatoid arthritis, systemic lupus erythematosus, Sjogren's syndrome, lymphohistocytosis, myocarditis, multiple sclerosis, autoimmune encephalomeyeltitis, insulin-dependent diabetes mellitus, allergies, allograft rejection, xeno transplant rejection and/or graft versus host disease.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention also relates to the use of a binder, amino acid sequence, Nanobody, compound or polypeptide of the invention in the preparation of a pharmaceutical composition for the prevention and/or treatment of at least one disease or disorder that can be prevented and/or treated by administering a binder, amino acid sequence. Nanobody, compound or polypeptide of the invention to a patient.

Again, in such a pharmaceutical composition, the one or more binders, amino acid sequences, Nanobodies, compounds or polypeptides of the invention may also be suitably combined with one or more other active principles, such as those mentioned herein.

Further uses of the binders, amino acid sequences, Nanobodies, compounds, polypeptides, nucleic acids, genetic constructs and hosts and host cells of the invention will be clear to the skilled person based on the disclosure herein. For example, and without limitation, the binders, amino acid sequences or Nanbodies of the invention can be linked to a suitable carrier or solid support so as to provide a medium than can be used in a manner known per se to purify a first and/or second naturally occurring binding molecule from compositions and preparations comprising the same. Derivatives of the amino acid sequences of the invention that comprise a suitable detectable label can also be used as markers to determine (qualitatively or quantitatively) the presence of a first and/or second naturally occurring binding molecule in a composition or preparation or as a marker to selectively detect the presence of a first and/or second naturally occurring binding molecule on the surface of a cell or tissue (for example, in combination with suitable cell sorting techniques).

The invention will now be further described by means of the following non-limiting examples:

### FIGURES

FIG. 1: Binding of selected B7-H1 binding Nanobodies to B7-H1 in ELISA.

FIG. 2: Binding of selected PD-L2 binding Nanobodies to PD-L2 in ELISA.

FIG. 3: Binding of selected B7-H1 binding Nanobodies to PD-L2 in ELISA.

FIG. 4: Binding of selected PD-L2 binding Nanobodies to B7-H in ELISA

### EXAMPLES

### Example 1

### Materials

A fusion protein consisting of the extracellular part of human PD-1 and mouse Fc gamma 1 was obtained from R&D Systems as a recombinant protein produced in NSO cells (Cat #1086-PD).

A fusion protein consisting of the extracellular part of human PD-L2 and mouse Fc gamma 1 was obtained from R&D Systems as a recombinant protein produced in NSO cells (Cat #224-PL).

A fusion protein consisting of the extracellular part of human B7-H1 (PD-L1) and mouse Fc gamma 1 was obtained from R&D Systems as a recombinant protein produced in NSO cells (Cat #156-B7).

### Example 2

### Immunizations with B7-H1 (PD-L1)

One llama (No. 149) was immunized with 6 boosts (100 or 50 μg/dose at weekly intervals) of R&D Systems (Minneapolis, Minn., US) Cat #156-B7, which is the ectodomain of human B7-1-11 (rh B7H1-Fc), formulated in Titermax Gold (Titermax USA, Norcross, Ga., US), according to standard protocols. At week 4, sera were collected to define antibody titers against B7-H1 by ELISA. In short, 96-well Maxisorp plates (Nunc, Wiesbaden, Germany) were coated with rh B7H1-Fc. After blocking and adding diluted sera samples, the presence of anti-B7-H1 Nanobodies was demonstrated by using rabbit anti-llama immunoglobulin antiserum and anti-rabbit immunoglobulin alkaline phosphatase conjugate. The titer exceeded 16000.

### Example 3

### Library Construction

Peripheral blood mononuclear cells were prepared from blood samples obtained from llama No. 149 using Ficoll-Hypaque according to the manufacturer's instructions (Amersham Biosciences, Uppsala, Sweden). Next, total RNA was extracted from these cells and used as starting material for RT-PCR to amplify Nanobody encoding gene fragments. These fragments were cloned into an expression vector derived from pUC119 which contained the LacZ promoter, a coliphage pill protein coding sequence, a resistance gene for ampicillin or carbenicillin, a multicloning site and the gen3 leader sequence. In frame with the Nanobody coding sequence, the vector coded for a C-terminal c-myc tag and a (His)6 tag. Phage was prepared according to standard methods (see for example the prior art and applications filed by applicant cited herein) and stored after filter sterilization at 4° C. for further use.

### Example 4

### Selections of B7-H1 (PD-L1) Binding Nanobodies

The phage library obtained from llamas No. 149 was used for 2 rounds of phage display selection.

In a first round, rhB7H1-Fc (R&D Systems, Minneapolis, US, Cat #156-B7) or rhPDL2-Fc (R&D Systems, Minneapolis, US, Cat #1224-PL) was coated onto Maxisorp 96-well plates (Nunc, Wiesbaden, Germany) at 0.5 and 5 μg/m. Preincubation of the phages with total human IgG (100 μg/ml) in 2% marvel PBST was followed by incubation with the phage libraries and extensive washing. Bound phage was aspecifically eluted with trypsin (1 mg/ml in PBS) or specifically eluted with PD-1 (100 μg/ml) or with BSA (100 μg/ml) as a control. Enrichment was observed over non-coated wells and wells aspecifically coated with rhPDL2-Fc.

In a second round, rhB7H1-Fc (R&D Systems, Minneapolis, US, Cat #156-B7) was coated onto Maxisorp 96-well plates (Nunc, Wiesbaden, Germany) at 0.5 and 5 μg/m. Bound phage was aspecifically eluted with trypsin (1 mg/ml in PBS) or specifically eluted with PD-1 (100 μg/ml) or with BSA (100 μg/ml) as a control. After this second round of selection, high enrichment was observed.

The output from the selection were plated onto LB/amp/2% glu plates. Colonies were picked and grown in 96 deep well plates (1 ml volume) and induced by adding IPTG for Nanobody expression. Periplasmic extracts (volume: ˜80 μl) were prepared according to standard methods (see for example the prior art and applications filed by applicant cited herein). The sequences of the clones obtained are depicted in Table B-1.

### Example 5

### Binding of the Obtained Nanobodies to PD-L1 in ELISA

In order to determine binding specificity to B7-H1 by the Nanobodies obtained from the selection described in Example 4, 96 eluted clones were tested in an ELISA binding assay setup.

In short, 5 μg/ml B7-H1 ectodomain (rhB7H1-Fc, R&D Systems, Minneapolis, US, Cat #156-B7) or control Fc was immobilized on maxisorp microtiter plates (Nunc, Wiesbaden, Germany) and free binding sites were blocked using 4% Marvel in. PBS. Next, 10 μl of periplasmic extract containing Nanobody of the different clones in 100 μl 2% Marvel PBST were allowed to bind to the immobilized antigen. After incubation and a wash step, Nanobody binding was revealed using a mouse-anti-myc secondary antibody, which was after a wash step detected with a HRP-conjugated donkey-anti-mouse antibody. Binding specificity was determined based on OD values compared to controls having received no Nanobody (low control). 17 out of the 96 selected clones were able to bind to B7-H1 with some specificity. 1 clone was shown to bind to the Fc part of the B7-H1-Fc-fusion as it also yielded high OD values in the parallel Fc control ELISA.

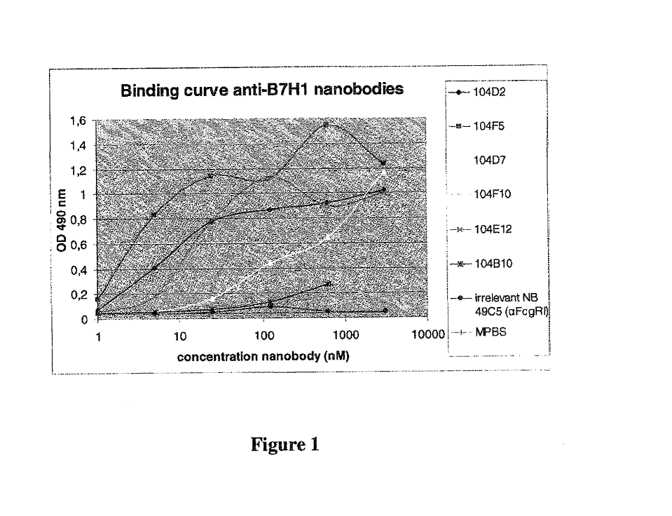

Based on these binding data, clones were selected for recloning in an expression vector derived from pUC119 which contained the LacZ promoter, a resistance gene for ampicillin or carbenicillin, a multicloning site and the gen3 leader sequence. In frame with the Nanobody coding sequence, the vector coded for a C-terminal c-myc tag and a (His)6 tag. After expression, the obtained Nanobodies were purified via the His-tag on Talon beads. Purified Nanobodies were again tested for binding B7-H1 in the ELISA binding assay as described above. OD values are shown in FIG. 1.

### Example 6

### Immunizations with PD-L2

One llama (No. 149) was immunized with 6 boosts (100 or 50 μg/dose at weekly intervals) of R&D Systems (Minneapolis, Minn., US) Cat #1224-PL, which is the ectodomain of human PD-L2 (rhPDL2-Fc), formulated in Titermax Gold (Titermax USA, Norcross, Ga., US), according to standard protocols. At week 4, sera were collected to define antibody titers against PD-L2 by ELISA. In short, 96-well Maxisorp plates (Nunc Wiesbaden, Germany) were coated with rhPDL2-Fc. After blocking and adding diluted sera samples, the presence of anti-PD-L2 Nanobodies was demonstrated by using rabbit anti-llama immunoglobulin antiserum and anti-rabbit immunoglobulin alkaline phosphatase conjugate. The titer exceeded 16000.

### Example 7

### Library Construction

Peripheral blood mononuclear cells were prepared from blood samples obtained from llama No. 149 using Ficoll-Hypaque according to the manufacturer's instructions (Amersham Biosciences, Uppsala, Sweden). Next, total RNA extracted was extracted from these cells and used as starting material for RT-PCR to amplify Nanobody encoding gene fragments. These fragments were cloned into an expression vector derived from pUC119 which contained the LacZ promoter, a coliphage pIII protein coding sequence, a resistance gene for ampicillin or carbenicillin, a multicloning site and the gen3 leader sequence. In frame with the Nanobody coding sequence, the vector coded for a C-terminal c-myc tag and a (His)6 tag. Phage was prepared according to standard methods (see for example the prior art and applications filed by applicant cited herein) and stored after filter sterilization at 4° C. for further use.

### Example 8

### Selection of PD-L2 Binding Nanobodies

The phage library obtained from llamas No 149 was used for 2 rounds of phage display selection.

In a first round, rhB7H1-Fc (R&D Systems, Minneapolis, US, Cat #156-B7) or rhPDL2-Fc (R&D Systems, Minneapolis, US, Cat #1224-PL) was coated onto Maxisorp 96-well plates (Nunc, Wiesbaden, Germany) at 0.5 and 5 μg/m. Preincubation of the phages with total human IgG (100 μg/ml) in 2% marvel PBST was followed by incubation with the phage libraries and extensive washing. Bound phage was aspecifically eluted with trypsin (1 mg/ml in PBS) or specifically eluted with PD-1 (100 μg/ml) or with BSA (100 μg/ml) as a control. Enrichment was observed over non-coated wells and control wells coated with rhPDL1-Fc.

In a second round, rhB7H2-Fc (R&D Systems, Minneapolis, US, Cat #1224-PL) was coated onto Maxisorp 96-well plates (Nunc, Wiesbaden, Germany) at 0.5 and 5 μg/m. Bound phage was aspecifically eluted with trypsin (1 mg/ml in PBS), specifically eluted with PD-1 (100 μg/ml), or with BSA (100 μg/ml) as a control. After this second round of selection, high enrichment was observed.

The output from the selection were plated onto LB/amp/2% glu plates. Colonies were picked and grown in 96 deep well plates (1 ml volume) and induced by adding IPTG for Nanobody expression. Periplasmic extracts (volume: ˜80 μl) were prepared according to standard methods (see for example the prior art and applications filed by applicant cited herein). The sequences of the clones obtained are depicted in Table B-2.

### Example 9

### Binding of the Obtained Nanobodies to PD-L2 in ELISA

In order to determine binding specificity to PD-L2 by the Nanobodies obtained from the selection described in Example 8, 96 eluted clones were tested in an ELISA binding assay setup.

In short, 5 μg/ml PD-L2 ectodomain (R&D Systems, Minneapolis, US, Cat #1224-PL) was immobilized on maxisorp microtiter plates (Nunc, Wiesbaden, Germany) and free binding sites were blocked using 4% Marvel in PBS. Next, 10 μl of periplasmic extract containing Nanobody of the different clones in 100 μl 2% Marvel PBST were allowed to bind to the immobilized antigen. After incubation and a wash step, Nanobody binding was revealed using a mouse-anti-myc secondary antibody, which was after a wash step detected with a HRP-conjugated donkey-anti-mouse antibody. Binding specificity was determined based on OD values compared to controls having received no Nanobody (low control). 32 out of the 96 selected clones were able to bind to PD-L2 with some specificity.

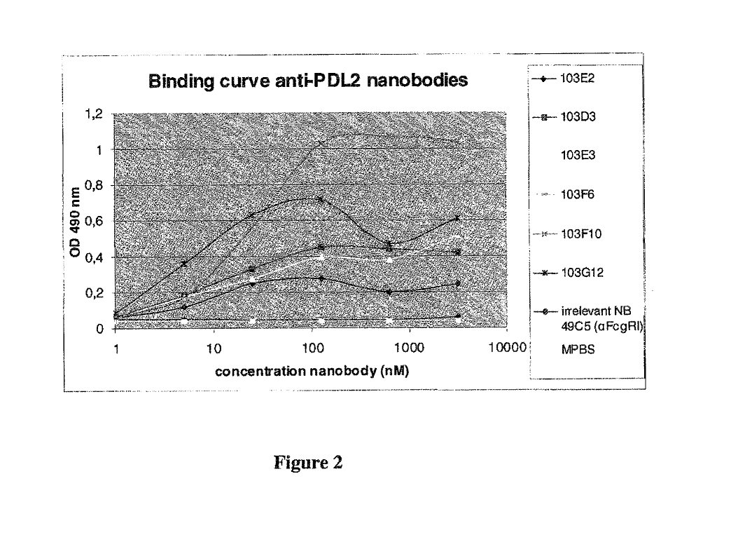

Clones were selected for recloning in an expression vector derived from pUC119 which contained the LacZ promoter, a resistance gene for ampicillin or carbenicillin, a multicloning site and the gen3 leader sequence. In frame with the Nanobody® coding sequence, the vector coded for a C-terminal c-myc tag and a (His)6 tag. After expression, the obtained Nanobodies were purified via the His-tag on Talon beads. Purified Nanobodies were tested in ELISA for binding to PD-L2 as described. Results are shown in FIG. 2.

### Example 10

### Multispecificity of the PD-L1 Binding Nanobodies

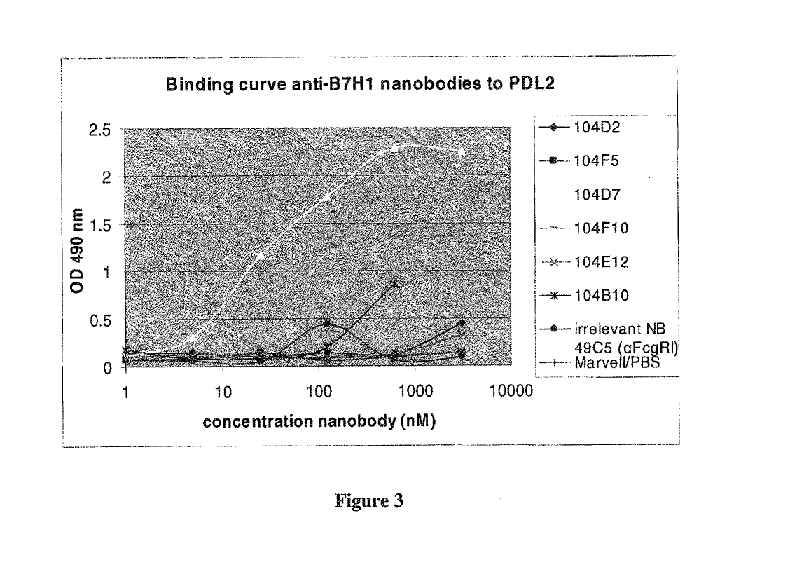

In order to check for bispecific binding, the clones selected and screened for B7-H1 binding were tested for binding PD-L2 in an ELISA setup. In short, plates were coated with 100 μl (2 μg/ml) of PDL2/hFc (R&D Systems, Minneapolis, US, Cat #1224-PL) in PBS overnight at 4C. After washing, plates were blocked with 4% MPBS for 2 hours on a shaker, A dilution series of Nanobody (1-3125 nM) in 100 μl 2% Marvell/PBS was added and incubated for 1 hour on a shaker. After washing, detection was performed with 100 μl 1:5000 rabbit-anti-V_{HH} in 2% Marvell/PBS for 1 hour on a shaker. After washing, anti-V_{HH} antibody was detected with 100 μl 1:5000 DARPO (in 2% Marvell/PBS for 1 hour on a shaker). PO was detected with 100 μl OPD (add 1:1000 H₂O₂ before use). The reaction was stopped with 50 μM H₂SO₄ and absorption was read at 490 nm with a platereader. Optical densities are shown in FIG. 3.

### Example 11

### Multispecificity of the PD-L2 Binding Nanobodies

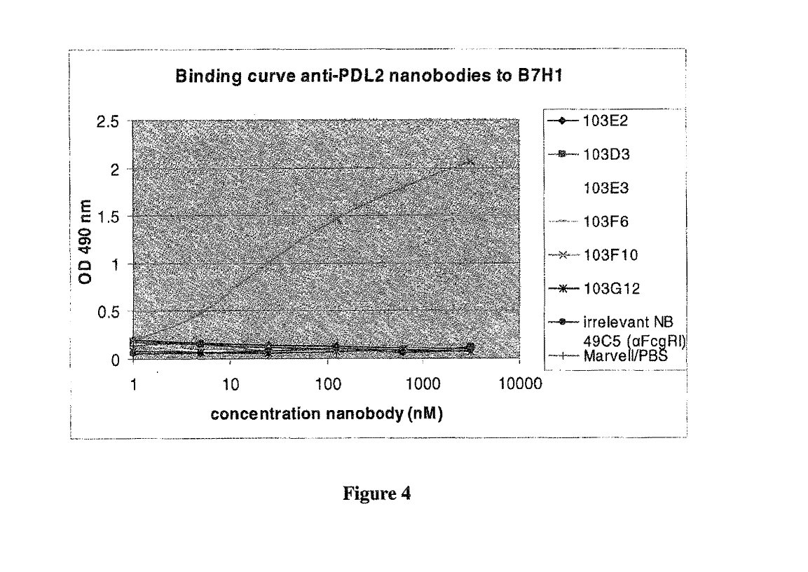

In order to check for bispecific binding, the clones selected and screened for PD-L2 binding were tested for binding PD-L1 in an ELISA setup. In short, plates were coated with 100 μl (2 μg/ml) of B7H1/hFc (R&D Systems, Minneapolis, US, Cat #156-B7) in PBS overnight at 4C. After washing, plates were blocked with 4% MPBS for 2 hours on a shaker. A dilution series of Nanobody (1-3125 nM) in 100 μl 2% Marvell/PBS was added and incubated for 1 hour on a shaker. After washing, detection was performed with 100 μl 1:5000 rabbit-anti-V_{HH} in 2% Marvell/PBS for 1 hour on a shaker. After washing, anti-V_{HH} antibody was detected with 100 μl 1:5000 DARPO (in 2% Marvell/PBS for 1 hour on a shaker). PO was detected with 100 μl OPD (add 1:1000 H₂O₂ before use). The reaction was stopped with 50 μl 1 M H₂SO₄ and absorption was read at 490 nm with a platereader. Optical densities are shown in FIG. 4.

### Tables

TABLE B-1
Preferred Nanobodies against PD-LI (B7-H1)

>104D2, SEQ ID NO: 25; PRT; ->
EVQLVESGGGLVQPGGSLRLSCAASGFTLDYYAIGWFRQAPGKEREWASS
ISSSDGSTYYADSVKGRFTISRDNAKNTVFLQMNSLKPEDTAVYSCAASQ
APITIATMMKPFYDYWGQGTQVTVSS
>104F5, SEQ ID NO: 26; PRT; ->
EVQLVESGGGLVQPGGSLRLSCAASGFTLDYYAKCWFRQAPGKEREWVSC
ISSSDGSTYYADSVKGRFTISRDNAKNTVYLQMNSLKPEDTAVYFCAARH
GGPLTVEYFFDYWGQGTQVTVSS
>104E12, SEQ ID NO: 27; PRT; ->
EVQLVESGGGLVQPGGSLRLSCAASGFTFDYYAIGWFRQAPGKAREGVSC
ISGGDNSTYYADSVKGRFTISRDNAKNTVYLQMNSLKPEDTAVYYCATGG
WKYCSGYDPEYTYWGQGTQVTVSS
>104B10, SEQ ID NO: 28; PRT; ->
EVQLVESGGGLVQAGGSLRLSCAASGSTFSQYDVGWYRQAPGKQRELVAF
SSSGGRTIYPDSVKGRFTFSRDNTKNTVYLQMTSLKPEDTAVYYCKIDWY
LNSYWGQGTQVTVSS
>104F10, SEQ ID NO: 23; PRT; ->
EVQLVESGGGLVQAGGSLRLSCAASGVDASNSAMGWYRQAPGKQREWVAR
ITGGGLIAYTDSVKGRFTISRDNAKSTVYLQMNSLEPEDTAVYYCNTINS
RDGWGQGTQVTVSS
>104D7, SEQ ID NO: 22; PRT; ->
EVQLVESGGGLVQAGGSLTISCAASGITFSDSIVSWYRRARGKQREWVAG
ISNGGTTKYAESVLGRFTISRDNAKNMVYLQMNGLNPEDTAVYLCKVRQY
WGQGTQVTVSS

TABLE B-2
Preferred Nanobodies against PD-L2

>103E2, SEQ ID NO: 29; PRT; ->
EVQLVESGGGLVQAGGSLRLSCAASGSTFSNYVSNYAMGWGRQAPGTQRE
LVASISNGDTTNYADSVKGRFTISRDNAKNTVYLQMNSLKPEDTAVYYCF
EHQVAGLTWGQGTQVTVSS
>103G12, SEQ ID NO: 33; PRT; ->
EVQLVESGGGLVQAGGSLRLSCVASGXALKIXVMGWYRQAPGKQRELVAA
ITSGGRTNYSDSVKGRFTISGDNAXNTVYLQMNSLKSEDTAVYYCREWNS
GYPPVDYWGQGTQVTVSS
>103F10, SEQ ID NO: 24; PRT; ->
EVQLVESGGGLVQAGGSLRLSCAASGRTFSSGTMGWFRRAPGKEREFVAS
IPWSGGRTYYADSVKDRFTISRDNAQNTVFLQMNSLKPEDTAVYYCAFKE
RSTGWDFASWGQGIQVTVSS
>103E3, SEQ ID NO: 31; PRT; ->
EVQEVESGGGLVQTGGSLRLSCAASGFTLDYYGIGWFRQAPGKEREGVSF
ISGSDGSTYYAESVKGRFTISRDKAKNTVYLQMNSLKPEDTAVYYCAADP
WGPPSIATMTSYEYNHWGQGTQVTVSS
>103F6, SEQ ID NO: 32; PRT; ->
EVQLVESGGGLVQPGGSLRLSCAASGFTFSTYTMIWLRRAPGKGFEWVST
IDKDGNTNYVDSVKGRFAVSRDNTKNTLYLQMNSLKPEDTAMYYCTKHGS
SARGQGTRVTVSS
>103D3, SEQ ID NO: 30; PRT; ->
EVQLVESGGGLVEPGGSLRLSCVASGFTFSSYDMSWVRQAPGKGLEWVST
INSGGGITYRGSVKGRFTISRDNAKNTLYLQMNSLKPEDTAVYYCENGGS
SYRRGQGTQVTVSS

TABLE B-3
Leader sequences and N-terminal sequences
<Name, SEQ ID #; PRT (protein); ->
Sequence

> llama leader 1, SEQ ID NO:; PRT; ->
VNKLLFAIPLVVPFYAAQPAMA
< llama leader 2, SEQ ID NO:; PRT; ->
VKKLLFAIPLVVPFYAAQPAIA
< leader sequence, SEQ ID NO:; PRT; ->
MKKTAIAIAVALAGLATVAQA
< leader sequence, SEQ ID NO:; PRT; ->
MKKTAIAFAVALAGLATVAQA
< N-terminal sequence, SEQ ID NO:; PRT; ->
AAAEQKLISEEDLNGAAHHHHHH

1. A single variable domain directed against at least two naturally occurring binding molecules, wherein said at least two naturally occurring binding molecules interact with the same third naturally occurring binding molecule.

2. (canceled)

3. The single variable domain directed against at least two naturally occurring binding molecules according to claim 1, wherein the stretch of amino acids on said single variable domain that interacts with the first naturally occurring binding molecule partially or fully overlaps in primary and/or tertiary structure with the stretch of amino acids that interacts with the second naturally occurring binding molecule.

4.-17. (canceled)

18. The single variable domain directed against at least two naturally occurring binding molecules according to claim 1, wherein the dissociation constant (K_{d}) in the binding of said single variable domain to the first naturally occurring binding molecule approximates the K_{d} in the binding of said single variable domain to the second naturally occurring binding molecule.

19.-23. (canceled)

24. The single variable domain according to claim 1, directed against at least two naturally occurring binding molecules, wherein said at least two naturally occurring binding molecules are binding molecules naturally occurring in humans.

25. The single variable domain according to claim 1, directed against at least two naturally occurring binding molecules, wherein said at least two naturally occurring binding molecules are involved in the same or similar biological pathways and/or biological mechanisms.

26. The single variable domain according to claim 1, directed against at least two naturally occurring binding molecules, wherein said at least two naturally occurring binding molecules interact with a third naturally occurring binding molecule that (mainly) occurs in circulation.

27. The single variable domain according to claim 1, directed against at least two naturally occurring binding molecules, wherein said at least two naturally occurring binding molecules interact with a third naturally occurring binding molecule that is a protein or peptide.

28. The single variable domain according to claim 1, directed against at least two naturally occurring binding molecules, wherein said at least two naturally occurring binding molecules interact with a third naturally occurring binding molecule that is an agonist for both the first naturally occurring binding molecule and the second naturally occurring binding molecule (or the biological action or mechanism in which the first naturally occurring binding molecule and the second naturally occurring binding molecule are involved).

29. The single variable domain according to claim 1, directed against at least two naturally occurring binding molecules, wherein said at least two naturally occurring binding molecules interact with a third naturally occurring binding molecule that is an antagonist for both the first naturally occurring binding molecule and the second naturally occurring binding molecule (or the biological action or mechanism in which the first naturally occurring binding molecule and the second naturally occurring binding molecule are involved).

30. The single variable domain according to claim 1, directed against at least two naturally occurring binding molecules, wherein said at least two naturally occurring binding molecules interact with a third naturally occurring binding molecule that belongs to one of the following classes of biological molecules: cytokines, hormones and chemokines.

31. The single variable domain according to claim 1, directed against at least two naturally occurring binding molecules, wherein said at least two naturally occurring binding molecules are involved in modulating cellular responses to the third naturally occurring binding molecule.

32. The single variable domain according to claim 1, directed against at least two naturally occurring binding molecules, wherein said at least two naturally occurring binding molecules are involved in the immune system and/or in modulating the immune system.

33. The single variable domain according to claim 1, directed against at least two naturally occurring binding molecules, wherein said at least two naturally occurring binding molecules are both located on one or more of the following cells: antigen presenting cells (APC), T-cells, B-cells, Natural killer (NK) cells, macrophages, Dendritic (DC) cells, parenchymal cells, splenocytes, thymocytes, monocytes, lymphoid cells, tumor cells, granulocytes, endothelial cells, epithelial cells, osteoblasts, skin cells, lung cells, colon cells, fibroblasts, Reed-Sternberg cells, peripheral blood lymphocytes, non-lymphoid haematopoietic cells, stromal cells, osteoclasts, hair follicles and brain cells and neurons.

34. The single variable domain directed against at least two naturally occurring binding molecules according to claim 33, wherein said cell is selected from the group consisting of antigen presenting cells (APC) and T-cells.

35. The single variable domain according to claim 1, directed against at least two naturally occurring binding molecules, wherein said at least two naturally occurring binding molecules are receptors or ligands.

36. The single variable domain according to claim 1, directed against at least two naturally occurring binding molecules, wherein said at least two naturally occurring binding molecules belong to the same protein family or superfamily.

37. The single variable domain according to claim 36, wherein said at least two naturally occurring binding molecules belong to the TNF superfamily, the TNFR superfamily, the B7:CD28 superfamily or Eph family.

38. The single variable domain according to claim 1, directed against at least two naturally occurring binding molecules, wherein said at least two naturally occurring binding molecules interact with one of the following third naturally occurring binding molecules:
TNFα
TNFβ
CD95L
VEGI
TRAIL
RANKL
LIGHT
APRIL
BAFF
TNFR1
TNFR2
DCR3
OPG
LβR
HVEM
BCMA
TACI
CD28
CTLA4
PD-1
CD80
CD86
MHC
EphA1
EphA2
EphA3
EphA4
EphA5
EphA6
EphA7
EphA8
EphB1
EphB2
EphB3
EphB4
EphB5
EphB6
ephrinA1
ephrinA2
ephrinA3
ephrinA4
ephrinA5
ephrinA6
ephrinB1
ephrinB2
ephrinB3.

39. The single variable domain according to claim 37, wherein said at least two naturally occurring binding molecules are selected from one of the following combinations of first and second naturally occurring binding molecules:
TNFR1, TNFR2 and HVEM;
CD95 and DCR3;
DCR3 and DR3;
DR4, DR5, DCR1, DCR2 and OPG;
OPG and RANKL;
LTβR, DR3 and HVEM;
BCMA, TACI and BAFFR;
TNFα and TNFβ;
CD95L and VEGI;
VEGI and LIGHT;
TRAIL and RANKL;
LTα/LTβ and LIGHT;
TNFβ and LIGHT;
APRIL and BAFF
CD80 and CD86
PD-L1 and PD-L2
CD28 and CTLA-4
TCRαβ and CD4/CD8
Eph A1, Eph A2, Eph A3, Eph A4, Eph A5, Eph A6, Eph A7, Eph A8
Eph B1, Eph B2, Eph B3, Eph B4, Eph B5, Eph B6
ephrinA1, ephrinA2, ephrinA3, ephrinA4, ephrinA5, ephrinA6
ephrinB1, ephrinB2, ephrinB3.

40. The single variable domain according to claim 39, wherein said at least two naturally occurring binding molecules are PD-L1 and PD-L2.

41. The single variable domain according to claim 1, which inhibits and/or blocks the interaction between said at least two naturally occurring binding molecules and said third naturally occurring binding molecule.

42. The single variable domain according to claim 41, which inhibits and/or blocks one of the following interactions:
TNFα with TNFR1 and TNFR2;
TNFα with TNFR1, TNFR2 and/or HVEM;
CD95L with CD95 and DCR3;
VEGI with DCR3 and DR3;
TRAIL with DR4, DR5, DCR1, DCR2 and/or OPG;
RANKL with OPG and RANKL;
LIGHT with LTβR, DR3 and/or HVEM;
APRIL with BCMA and TACI;
BAFF with BCMA, TACI and/or BAFFR;
TNFR1 with TNFα and TNFβ;
TNFR2 with TNFα and TNFβ;
DCR3 with CD95L and VEGI;
DR3 with VEGI and LIGHT;
OPG with TRAIL and RANKL;
LTβR with LTα/LTβ and LIGHT;
HVEM with TNFβ and LIGHT;
BCMA with APRIL and BAFF;
TACI with APRIL and BAFF;
CD28 with CD80 and CD86;
CTLA-4 with CD80 and CD86;
PD-1 with PD-L1 and PD-L2;
CD80 with CD28 and CTLA-4;
CD86 with CD28 and CTLA-4;
MHC with TCRαβ and CD4/CD8
EphA1 with ephrinA1, ephrinA2, ephrinA3, ephrinA4, and/or ephrinA5
EphA2 with ephrinA1, ephrinA2, ephrinA3, ephrinA4, ephrinA5, and/or ephrin A6
EphA3 with ephrinA1, ephrinA2, ephrinA3, ephrinA4, ephrinA5, and/or ephrin A6
EphA4 with ephrinA1, ephrinA2, ephrinA3, ephrinA4, ephrinA5, ephrin A6, ephrinB2 and/or ephrinB3
EphA5 with ephrinA1, ephrinA2, ephrinA3, ephrinA4, ephrinA5, and/or ephrin A6
EphA6 with ephrinA1, ephrinA2, ephrinA3, ephrinA4, ephrinA5, and/or ephrin A6
EphA7 with ephrinA1, ephrinA2, ephrinA3, ephrinA4, ephrinA5, and/or ephrin A6
EphA8 with ephrinA1, ephrinA2, ephrinA3, ephrinA4, ephrinA5, and/or ephrin A6
EphB1 with ephrinB1, ephrinB2, and/or ephrinB3
EphB2 with ephrinB1, ephrinB2, and/or ephrinB3
EphB3 with ephrinB1, ephrinB2, and/or ephrinB3
EphB4 with ephrinB1, ephrinB2, and/or ephrinB3
EphB5 with ephrinB1, ephrinB2, and/or ephrinB3
EphB6 with ephrinB1, ephrinB2, and/or ephrinB3
ephrinA1 with EphA1, EphA2, EphA3, EphA4, EphA5, EphA6, EphA7, and/or EphA8
ephrinA2 with EphA1, EphA2, EphA3, EphA4, EphA5, EphA6, EphA7, and/or EphA8
ephrinA3 with EphA1, EphA2, EphA3, EphA4, EphA5, EphA6, EphA7, and/or EphA8
ephrinA4 with EphA1, EphA2, EphA3, EphA4, EphA5, EphA6, EphA7, and/or EphA8
ephrinA5 with EphA1, EphA2, EphA3, EphA4, EphA5, EphA6, EphA7, and/or EphA8
ephrinA6 with EphA2, EphA3, EphA4, EphA5, EphA6, EphA7, and/or EphA8
ephrinB1 with EphB1, EphB2, EphB3, EphB4, EphB5, and/or EphB6
ephrinB2 with EphB1, EphB2, EphB3, EphB4, EphB5, and/or EphB6
ephrinB3 with EphB1, EphB2, EphB3, EphB4, EphB5, and/or EphB6,

43. (canceled)

44. The single variable domain according to claim 42, which inhibits and/or blocks the interaction of PD-L1 and PD-L2 with PD-1.

45.-49. (canceled)

50. The single variable domain according to claim 1, which essentially consists of a light chain variable domain sequence (e.g. a V_{L}-sequence) or a suitable fragment thereof, or of a heavy chain variable domain sequence (e.g. a V_{H}-sequence) or a suitable fragment thereof.

51. The single variable domain according to claim 1, which essentially consists of a heavy chain variable domain sequence that is derived from a conventional four-chain antibody or which essentially consist of a heavy chain variable domain sequence that is derived from a heavy chain antibody or a suitable fragment thereof.

52. The single variable domain according to claim 1, which essentially consists of a domain antibody (or an amino acid sequence that is suitable for use as a domain antibody), of a single domain antibody (or an amino acid sequence that is suitable for use as a single domain antibody), of a “dAb” (or an amino acid sequence that is suitable for use as a dAb) or of a Nanobody® (including but not limited to a V_{HH} sequence), or any suitable fragment of any one thereof.

53.-60. (canceled)

61. Single variable domain Amino acid sequence directed against at least two naturally occurring binding molecules, that essentially consists of 4 framework regions (FR1 to FR4, respectively) and 3 complementarity determining regions (CDR1 to CDR3, respectively), in which:
CDR1 is chosen from the group consisting of:
a) the amino acid sequence of SEQ ID NO's: 4-6;
b) amino acid sequences that have at least 80% amino acid identity with the amino acid sequence of SEQ ID NO's: 4-6;
c) amino acid sequences that have 3, 2, or 1 amino acid difference with the amino acid sequence of SEQ ID NO's: 4-6;
and/or
CDR2 is chosen from the group consisting of
d) the amino acid sequences of SEQ ID NO's: 10-12;
e) amino acid sequences that have at least 80% amino acid identity with at least one of the amino acid sequences of SEQ ID NO's: 10-12;
f) amino acid sequences that have 3, 2, or 1 amino acid difference with at least one of the amino acid sequences of SEQ ID NO's: 10-12;
and/or
CDR3 is chosen from the group consisting of
g) the amino acid sequences of SEQ ID NO's: 16-18;
h) amino acid sequences that have at least 80% amino acid identity with at least one of the amino acid sequences of SEQ ID NO's: 16-18;
i) amino acid sequences that have 3, 2, or 1 amino acid difference with at least one of the amino acid sequences of SEQ ID NO's: 16-18; or any suitable fragment of such an amino acid sequence,

62.-107. (canceled)

108. Single variable domain according to claim 52, that is a naturally occurring Nanobody (from any suitable species) or a synthetic or semi-synthetic Nanobody.

109. Single variable domain according to claim 108, that is a V_{HH} sequence, a partially humanized V_{HH} sequence, a fully humanized V_{HH} sequence, a camelized heavy chain variable domain or a Nanobody that has been obtained by techniques such as affinity maturation.

110.-114. (canceled)

115. Single variable domain according to claim 108, that is chosen from the group consisting of SEQ ID NO's: 22-24 or from the group consisting of from amino acid sequences that have more than 80%, preferably more than 90%, more preferably more than 95%, such as 99% or more sequence identity (as defined herein) with at least one of the amino acid sequences of SEQ ID NO's: 22-24.

116. (canceled)

117. Compound or construct, that comprises or essentially consists of one or more single variable domains according to claim 1, and optionally further comprises one or more other groups, residues, moieties or binding units, optionally linked via one or more linkers.

118. Compound or construct according to claim 117, in which said one or more other groups, residues, moieties or binding units are amino acid sequences.

119. Compound or construct according to claim 117, in which said one or more linkers, if present, are one or more amino acid sequences.

120. Compound or construct according to claim 117, in which said one or more other groups, residues, moieties or binding units are immunoglobulin sequences.

121. Compound or construct according to claim 117, in which said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies.

122.-125. (canceled)

126. Compound or construct according to claim 117, which has an increased half-life, compared to the corresponding one or more single variable domains per se.

127. Compound or construct according to claim 126, in which said one or more other groups, residues, moieties or binding units provide the compound or construct with increased half-life, compared to the corresponding one or more single variable domains per se.

128. Compound or construct according to claim 127, in which said one or more other groups, residues, moieties or binding units that provide the compound or construct with increased half-life is chosen from the group consisting of serum proteins or fragments thereof, binding units that can bind to serum proteins, an Fc portion, and small proteins or peptides that can bind to serum proteins.

129. Compound or construct according to claim 127, in which said one or more other groups, residues, moieties or binding units that provide the compound or construct with increased half-life is chosen from the group consisting of human serum albumin or fragments thereof.

130. Compound or construct according to claim 127, in which said one or more other groups, residues, moieties or binding units that provides the compound or construct with increased half-life are chosen from the group consisting of binding units that can bind to serum albumin (such as human serum albumin) or a serum immunoglobulin (such as IgG).

131. Compound or construct according to claim 127, in which said one or more other groups, residues, moieties or binding units that provides the compound or construct with increased half-life are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies that can bind to serum albumin (such as human serum albumin) or a serum immunoglobulin (such as IgG).

132. Compound or construct according to claim 127, in which said one or more other groups, residues, moieties or binding units that provides the compound or construct with increased half-life is a Nanobody that can bind to serum albumin (such as human serum albumin) or a serum immunoglobulin (such as IgG).

133. Compound or construct according to claim 126, that has a serum half-life that is at least 1.5 times, preferably at least 2 times, such as at least 5 times, for example at least 10 times or more than 20 times, greater than the half-life of the corresponding one or more single variable domains.

134. Compound or construct according to claim 126, that has a serum half-life that is increased with more than 1 hours, preferably more than 2 hours, more preferably more than 6 hours, such as more than 12 hours, or even more than 24, 48 or 72 hours, compared to the corresponding one or more single variable domains.

135. Compound or construct according to claim 126, that has a serum half-life in human of at least about 12 hours, preferably at least 24 hours, more preferably at least 48 hours, even more preferably at least 72 hours or more; for example, of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

136. Monovalent construct, comprising or essentially consisting of one single variable domain according to claim 1,

137.-138. (canceled)

139. Nucleic acid or nucleotide sequence, that encodes a single variable domain according to claim 1.

140. Nucleic acid or nucleotide sequence according to claim 139, that is in the form of a genetic construct.

141. Host or host cell that expresses, or that under suitable circumstances is capable of expressing, a single variable domain according to claim 1.

142. Method for generating a single variable domain according to claim 1, comprising at least the steps of:
a) providing a set, collection or library of single variable domains;
and
b) screening said set, collection or library of single variable domains for single variable domains that can bind to and/or have affinity for the first naturally occurring binding molecule;
c) screening said set, collection or library of single variable domains for single variable domains that can bind to and/or have affinity for the second naturally occurring binding molecule;
and
d) isolating the single variable domain(s) that can bind to and/or have affinity for said first and said second naturally occurring binding molecule.

143. Method according to claim 142, comprising the steps of:
a) providing a set, collection or library of single variable domains; and
b) screening said set, collection or library of single variable domains for single variable domains that can bind to and/or have affinity for the first naturally occurring binding molecule;
c) screening the single variable domains obtained in step b) for single variable domains that can bind to and/or have affinity for the second naturally occurring binding molecule;
and
d) isolating the single variable domain(s) that can bind to and/or have affinity for said first and said second naturally occurring binding molecule.

144. Method for generating a single variable domain according to claim 1, comprising at least the steps of:
a) providing a collection or sample of cells expressing single variable domains;
b) screening said collection or sample of cells for cells that express a single variable domain that can bind to and/or has affinity for the first naturally occurring binding molecule;
c) screening said collection or sample of cells for cells that express a single variable domain that can bind to and/or has affinity for the second naturally occurring binding molecule;
and
d) from the cell that expresses a single variable domain that can bind to and/or have affinity for the first and second naturally occurring binding molecule either (i) isolating said single variable domain; or (ii) isolating from said cell a nucleic acid sequence that encodes said single variable domain, followed by expressing said single variable domain.

145. Method according to claim 144, comprising the steps of:
a) providing a collection or sample of cells expressing single variable domains;
b) screening said collection or sample of cells for cells that express a single variable domain that can bind to and/or has affinity for the first naturally occurring binding molecule;
c) screening said cells obtained in b) for cells that express a single variable domain that can bind to and/or has affinity for the second naturally occurring binding molecule;
and
d) from the cell that expresses a single variable domain that can bind to and/or has affinity for the first and second naturally occurring binding molecule either (i) isolating said single variable domain; or (ii) isolating from said cell a nucleic acid sequence that encodes said single variable domain, followed by expressing said amino acid sequence.

146. Method for generating a single variable domain according to claim 1, comprising at least the steps of:
a) providing a set, collection or library of nucleic acid sequences encoding single variable domains;
b) screening said set, collection or library of nucleic acid sequences for nucleic acid sequences that encode a single variable domain that can bind to and/or has affinity for the first naturally occurring binding molecule;
c) screening said set, collection or library of nucleic acid sequences for nucleic acid sequences that encode a single variable domain that can bind to and/or has affinity for the second naturally occurring binding molecule;
and
d) isolating said nucleic acid sequence that encode a single variable domain that can bind to and/or has affinity for the first and second naturally occurring binding molecule, followed by expressing said single variable domain.

147. Method according to claim 146, comprising the steps of:
a) providing a set, collection or library of nucleic acid sequences encoding single variable domains;
b) screening said set, collection or library of nucleic acid sequences for nucleic acid sequences that encode a single variable domain that can bind to and/or has affinity for the first naturally occurring binding molecule;
c) screening said nucleic acid sequences obtained in b) for nucleic acid sequences that encode a single variable domain that can bind to and/or has affinity for the second naturally occurring binding molecule;
and
d) isolating said nucleic acid sequence that encode a single variable domain that can bind to and/or has affinity for the first and second naturally occurring binding molecule, followed by expressing said single variable domain.

148. Method for producing a single variable domain, said method at least comprising the steps of:
a) expressing, in a suitable host cell or host organism or in another suitable expression system, a nucleic acid or nucleotide sequence according to claim 139, or a genetic construct containing a nucleic acid or nucleotide sequence according to claim 139;
optionally followed by:
b) isolating and/or purifying the single variable domain thus obtained.

149. Method for producing a single variable domain, said method at least comprising the steps of:
a) cultivating and/or maintaining a host or host cell according to claim 141 under conditions that are such that said host or host cell expresses and/or produces at least one single variable domain,
optionally followed by:
b) isolating and/or purifying the single variable domain, thus obtained.

150. Composition, comprising at least one single variable domain according to claim 1.

151. Composition according to claim 150, which is a pharmaceutical composition.

152. Composition according to claim 151, which is a pharmaceutical composition, that further comprises at least one pharmaceutically acceptable carrier, diluent or excipient and/or adjuvant, and that optionally comprises one or more further pharmaceutically active polypeptides and/or compounds.

153. Method for the prevention and/or treatment of at least one cancer, inflammatory disease or osteoporosis, said method comprising administering, to a subject in need thereof, a pharmaceutically active amount of at least one single variable domain according to claim 1.

154. Method according to claim 153, wherein the cancer is melanoma, a tumor, soft tissue sarcoma, skin cancer, drug-resistant bony sarcomas, and/or leukemia.

155. Method according to claim 153, wherein the inflammatory disease is Crohn's disease, rheumatoid arthritis, systemic lupus erythematosus, Sjogren's syndrome, lymphohistocytosis, myocarditis, multiple sclerosis, autoimmune encephalomyelitis, insulin-dependent diabetes mellitus, allergies, allograft rejection, xeno transplant rejection and/or graft versus host disease.

156. Method for the prevention and/or treatment of at least one disease or disorder that can be prevented and/or treated by administering, to a subject in need thereof, a single variable domain according to claim 1, said method comprising administering, to a subject in need thereof, a pharmaceutically active amount of at least one single variable domain according to claim 1.

157. Method for immunotherapy, said method comprising administering, to a subject in need thereof, a pharmaceutically active amount of at least one single variable domain according to claim 1.

158.-224. (canceled)


SEQUENCE LISTING (38 sequences)

SEQ ID NO: 1 - protein, 30 aa, Artificial Sequence
  Framework sequence
EVQLVESGGGLVQAGGSLTISCAASGITFS

SEQ ID NO: 2 - protein, 30 aa, Artificial Sequence
  Framework sequence
EVQLVESGGGLVQAGGSLRLSCAASGVDAS

SEQ ID NO: 3 - protein, 30 aa, Artificial Sequence
  Framework sequence
EVQLVESGGGLVQAGGSLRLSCAASGRTFS

SEQ ID NO: 4 - protein, 5 aa, Artificial Sequence
  CDR sequence
DSIVS

SEQ ID NO: 5 - protein, 5 aa, Artificial Sequence
  CDR sequence
NSAMG

SEQ ID NO: 6 - protein, 5 aa, Artificial Sequence
  CDR sequence
SGTMG

SEQ ID NO: 7 - protein, 14 aa, Artificial Sequence
  Framework sequence
WYRRARGKQREWVA

SEQ ID NO: 8 - protein, 14 aa, Artificial Sequence
  Framework sequence
WYRQAPGKQREWVA

SEQ ID NO: 9 - protein, 14 aa, Artificial Sequence
  Framework sequence
WFRRAPGKEREFVA

SEQ ID NO: 10 - protein, 16 aa, Artificial Sequence
  CDR sequence
GISNGGTTKYAESVLG

SEQ ID NO: 11 - protein, 16 aa, Artificial Sequence
  CDR sequence
RITGGGLIAYTDSVKG

SEQ ID NO: 12 - protein, 17 aa, Artificial Sequence
  CDR sequence
SIPWSGGRTYYADSVKD

SEQ ID NO: 13 - protein, 32 aa, Artificial Sequence
  Framework sequence
RFTISRDNAKNMVYLQMNGLNPEDTAVYLCKV

SEQ ID NO: 14 - protein, 32 aa, Artificial Sequence
  Framework sequence
RFTISRDNAKSTVYLQMNSLEPEDTAVYYCNT

SEQ ID NO: 15 - protein, 32 aa, Artificial Sequence
  Framework sequence
RFTISRDNAQNTVFLQMNSLKPEDTAVYYCAF

SEQ ID NO: 16 - protein, 3 aa, Artificial Sequence
  CDR sequence
RQY

SEQ ID NO: 17 - protein, 6 aa, Artificial Sequence
  CDR sequence
INSRDG

SEQ ID NO: 18 - protein, 11 aa, Artificial Sequence
  CDR sequence
KERSTGWDFAS

SEQ ID NO: 19 - protein, 11 aa, Artificial Sequence
  Framework sequence
WGQGTQVTVSS

SEQ ID NO: 20 - protein, 11 aa, Artificial Sequence
  Framework sequence
WGQGTQVTVSS

SEQ ID NO: 21 - protein, 11 aa, Artificial Sequence
  Framework sequence
WGQGIQVTVSS

SEQ ID NO: 22 - protein, 111 aa, Artificial Sequence
  Nanobody sequence
EVQLVESGGGLVQAGGSLTISCAASGITFSDSIVSWYRRARGKQREWVAGISNGGTTKYAESVLGRFTISRDNAKNMVYLQMNGLNPEDTAVYLCKVRQYWGQGTQVTVSS

SEQ ID NO: 23 - protein, 114 aa, Artificial Sequence
  Nanobody sequence
EVQLVESGGGLVQAGGSLRLSCAASGVDASNSAMGWYRQAPGKQREWVARITGGGLIAYTDSVKGRFTISRDNAKSTVYLQMNSLEPEDTAVYYCNTINSRDGWGQGTQVTVSS

SEQ ID NO: 24 - protein, 120 aa, Artificial Sequence
  Nanobody sequence
EVQLVESGGGLVQAGGSLRLSCAASGRTFSSGTMGWFRRAPGKEREFVASIPWSGGRTYYADSVKDRFTISRDNAQNTVFLQMNSLKPEDTAVYYCAFKERSTGWDFASWGQGIQVTVSS

SEQ ID NO: 25 - protein, 126 aa, Artificial Sequence
  Nanobody sequence
EVQLVESGGGLVQPGGSLRLSCAASGFTLDYYAIGWFRQAPGKEREWASSISSSDGSTYYADSVKGRFTISRDNAKNTVFLQMNSLKPEDTAVYSCAASQAPITIATMMKPFYDYWGQGTQVTVSS

SEQ ID NO: 26 - protein, 123 aa, Artificial Sequence
  Nanobody sequence
EVQLVESGGGLVQPGGSLRLSCAASGFTLDYYAKCWFRQAPGKEREWVSCISSSDGSTYYADSVKGRFTISRDNAKNTVYLQMNSLKPEDTAVYFCAARHGGPLTVEYFFDYWGQGTQVTVSS

SEQ ID NO: 27 - protein, 124 aa, Artificial Sequence
  Nanobody sequence
EVQLVESGGGLVQPGGSLRLSCAASGFTFDYYAIGWFRQAPGKAREGVSCISGGDNSTYYADSVKGRFTISRDNAKNTVYLQMNSLKPEDTAVYYCATGGWKYCSGYDPEYIYWGQGTQVTVSS

SEQ ID NO: 28 - protein, 115 aa, Artificial Sequence
  Nanobody sequence
EVQLVESGGGLVQAGGSLRLSCAASGSTFSQYDVGWYRQAPGKQRELVAFSSSGGRTIYPDSVKGRFTFSRDNTKNTVYLQMTSLKPEDTAVYYCKIDWYLNSYWGQGTQVTVSS

SEQ ID NO: 29 - protein, 119 aa, Artificial Sequence
  Nanobody sequence
EVQLVESGGGLVQAGGSLRLSCAASGSTFSNYVSNYAMGWGRQAPGTQRELVASISNGDTTNYADSVKGRFTISRDNAKNTVYLQMNSLKPEDTAVYYCFEHQVAGLTWGQGTQVTVSS

SEQ ID NO: 30 - protein, 114 aa, Artificial Sequence
  Nanobody sequence
EVQLVESGGGLVEPGGSLRLSCVASGFTFSSYDMSWVRQAPGKGLEWVSTINSGGGITYRGSVKGRFTISRDNAKNTLYLQMNSLKPEDTAVYYCENGGSSYRRGQGTQVTVSS

SEQ ID NO: 31 - protein, 127 aa, Artificial Sequence
  Nanobody sequence
EVQLVESGGGLVQTGGSLRLSCAASGFTLDYYGIGWFRQAPGKEREGVSFISGSDGSTYYAESVKGRFTISRDKAKNTVYLQMNSLKPEDTAVYYCAADPWGPPSIATMTSYEYKHWGQGTQVTVSS

SEQ ID NO: 32 - protein, 113 aa, Artificial Sequence
  Nanobody sequence
EVQLVESGGGLVQPGGSLRLSCAASGFTFSTYTMIWLRRAPGKGFEWVSTIDKDGNTNYVDSVKGRFAVSRDNTKNTLYLQMNSLKPEDTAMYYCTKHGSSARGQGTRVTVSS

SEQ ID NO: 33 - protein, 118 aa, Artificial Sequence
  Nanobody sequence
EVQLVESGGGLVQAGGSLRLSCVASGXALKIXVMGWYRQAPGKQRELVAAITSGGRTNYSDSVKGRFTISGDNAXNTVYLQMNSLKSEDTAVYYCREWNSGYPPVDYWGQGTQVTVSS

SEQ ID NO: 34 - protein, 22 aa, Artificial Sequence
  Leader sequence
VKKLLFAIPLVVPFYAAQPAMA

SEQ ID NO: 35 - protein, 22 aa, Artificial Sequence
  Leader sequence
VKKLLFAIPLVVPFYAAQPAIA

SEQ ID NO: 36 - protein, 21 aa, Artificial Sequence
  Leader sequence
MKKTAIAIAVALAGLATVAQA

SEQ ID NO: 37 - protein, 21 aa, Artificial Sequence
  Leader sequence
MKKTAIAFAVALAGLATVAQA

SEQ ID NO: 38 - protein, 23 aa, Artificial Sequence
  Leader sequence
AAAEQKLISEEDLNGAAHHHHHH